SEQUENCE LISTING (188 sequences)

SEQ ID NO: 1 - DNA, 30 bp, Artificial Sequence
  Primer
acgttggatgctcttatgttccagtttcag

SEQ ID NO: 2 - DNA, 30 bp, Artificial Sequence
  Primer
acgttggatggctgcctattccccactttg

SEQ ID NO: 3 - DNA, 30 bp, Artificial Sequence
  Primer
acgttggatgagatcaggagggtaatagac

SEQ ID NO: 4 - DNA, 30 bp, Artificial Sequence
  Primer
acgttggatggtcaataagcgttcttgccc

SEQ ID NO: 5 - DNA, 30 bp, Artificial Sequence
  Primer
acgttggatgtacagaagtgcttgtgtccc

SEQ ID NO: 6 - DNA, 30 bp, Artificial Sequence
  Primer
acgttggatgaatctgaggatgagctgaac

SEQ ID NO: 7 - DNA, 30 bp, Artificial Sequence
  Primer
acgttggatgaaaaaatggaccctgagtgc

SEQ ID NO: 8 - DNA, 30 bp, Artificial Sequence
  Primer
acgttggatgacctccgtaccatcaagtca

SEQ ID NO: 9 - DNA, 30 bp, Artificial Sequence
  Primer
acgttggatgcactataagctgggaacagg

SEQ ID NO: 10 - DNA, 30 bp, Artificial Sequence
  Primer
acgttggatgaggataacaccacccatacc

SEQ ID NO: 11 - DNA, 30 bp, Artificial Sequence
  Primer
acgttggatgacccggacaatgttgggaat

SEQ ID NO: 12 - DNA, 29 bp, Artificial Sequence
  Primer
acgttggatggggctaaggacatgaatag

SEQ ID NO: 13 - DNA, 30 bp, Artificial Sequence
  Primer
acgttggatggcaatttctctccgaaccac

SEQ ID NO: 14 - DNA, 30 bp, Artificial Sequence
  Primer
acgttggatggcaatcagtgctaatctccc

SEQ ID NO: 15 - DNA, 30 bp, Artificial Sequence
  Primer
acgttggatgagagatggggatcttctgag

SEQ ID NO: 16 - DNA, 29 bp, Artificial Sequence
  Primer
acgttggatgagctctggctgctcagtag

SEQ ID NO: 17 - DNA, 30 bp, Artificial Sequence
  Primer
acgttggatgctccagaaggccatttagtg

SEQ ID NO: 18 - DNA, 30 bp, Artificial Sequence
  Primer
acgttggatgcacaccgtgaatgaaccttc

SEQ ID NO: 19 - DNA, 30 bp, Artificial Sequence
  Primer
acgttggatgtgtgggccatgaatttgagg

SEQ ID NO: 20 - DNA, 30 bp, Artificial Sequence
  Primer
acgttggatgcactggtctctctgctatac

SEQ ID NO: 21 - DNA, 30 bp, Artificial Sequence
  Primer
acgttggatgtgtatcagtgaaggaatcgc

SEQ ID NO: 22 - DNA, 30 bp, Artificial Sequence
  Primer
acgttggatgcaaacccctattccatgctg

SEQ ID NO: 23 - DNA, 30 bp, Artificial Sequence
  Primer
acgttggatgcagatgatgggagctgtcac

SEQ ID NO: 24 - DNA, 30 bp, Artificial Sequence
  Primer
acgttggatgtgtaaggcatctggtggaga

SEQ ID NO: 25 - DNA, 30 bp, Artificial Sequence
  Primer
acgttggatgcatataagttagcgccagcc

SEQ ID NO: 26 - DNA, 30 bp, Artificial Sequence
  Primer
acgttggatggagcagatggttttaaggtg

SEQ ID NO: 27 - DNA, 30 bp, Artificial Sequence
  Primer
acgttggatgtcatctgcataggacagccc

SEQ ID NO: 28 - DNA, 30 bp, Artificial Sequence
  Primer
acgttggatgaagccatcctggaagacctg

SEQ ID NO: 29 - DNA, 30 bp, Artificial Sequence
  Primer
acgttggatgcaaagacccaacaacgcttg

SEQ ID NO: 30 - DNA, 30 bp, Artificial Sequence
  Primer
acgttggatgtgaatacagagaggcagcac

SEQ ID NO: 31 - DNA, 30 bp, Artificial Sequence
  Primer
acgttggatgggcatcatccccgaggaata

SEQ ID NO: 32 - DNA, 29 bp, Artificial Sequence
  Primer
acgttggatgtccgctggcgggcacggta

SEQ ID NO: 33 - DNA, 30 bp, Artificial Sequence
  Primer
acgttggatgaagaaaaggcaaaggatctg

SEQ ID NO: 34 - DNA, 31 bp, Artificial Sequence
  Primer
acgttggatgctgttggccatttctatgttg

SEQ ID NO: 35 - DNA, 29 bp, Artificial Sequence
  Primer
acgttggatgtgatgagatccaatttatc

SEQ ID NO: 36 - DNA, 29 bp, Artificial Sequence
  Primer
acgttggatgaggtaatattttcttggac

SEQ ID NO: 37 - DNA, 30 bp, Artificial Sequence
  Primer
acgttggatggcaaagctctcacgaggaag

SEQ ID NO: 38 - DNA, 30 bp, Artificial Sequence
  Primer
acgttggatgtctgcacacatcctgcttag

SEQ ID NO: 39 - DNA, 30 bp, Artificial Sequence
  Primer
acgttggatggatgccgggttgtaaatcac

SEQ ID NO: 40 - DNA, 30 bp, Artificial Sequence
  Primer
acgttggatgtttaccacctcatcgcatac

SEQ ID NO: 41 - DNA, 30 bp, Artificial Sequence
  Primer
acgttggatgctttgggagataggttctgc

SEQ ID NO: 42 - DNA, 30 bp, Artificial Sequence
  Primer
acgttggatgctggcttaaaagagggcttg

SEQ ID NO: 43 - DNA, 30 bp, Artificial Sequence
  Primer
acgttggatgctgcctaatacagggtcttc

SEQ ID NO: 44 - DNA, 30 bp, Artificial Sequence
  Primer
acgttggatgagagaagtggttttagactc

SEQ ID NO: 45 - DNA, 30 bp, Artificial Sequence
  Primer
acgttggatgaggagacaaaatcatctggg

SEQ ID NO: 46 - DNA, 30 bp, Artificial Sequence
  Primer
acgttggatgtgctgggattacagactgac

SEQ ID NO: 47 - DNA, 31 bp, Artificial Sequence
  Primer
acgttggatggcaaaaggactgataatgagc

SEQ ID NO: 48 - DNA, 31 bp, Artificial Sequence
  Primer
acgttggatggacaattaatattcccccctg

SEQ ID NO: 49 - DNA, 30 bp, Artificial Sequence
  Primer
acgttggatgcccggcctcaacaaatgtat

SEQ ID NO: 50 - DNA, 30 bp, Artificial Sequence
  Primer
acgttggatgagggccccagttttttagac

SEQ ID NO: 51 - DNA, 30 bp, Artificial Sequence
  Primer
acgttggatgaactaagggtgcctcatacg

SEQ ID NO: 52 - DNA, 30 bp, Artificial Sequence
  Primer
acgttggatgacaattagagagctaagcac

SEQ ID NO: 53 - DNA, 30 bp, Artificial Sequence
  Primer
acgttggatgctctacagtttacctaaggc

SEQ ID NO: 54 - DNA, 30 bp, Artificial Sequence
  Primer
acgttggatgtagtctaggcatgccagttg

SEQ ID NO: 55 - DNA, 30 bp, Artificial Sequence
  Primer
acgttggatgagctggtgacctagagatag

SEQ ID NO: 56 - DNA, 30 bp, Artificial Sequence
  Primer
acgttggatggacaaggctattccatcctc

SEQ ID NO: 57 - DNA, 30 bp, Artificial Sequence
  Primer
acgttggatggctgggtgattttagaggag

SEQ ID NO: 58 - DNA, 30 bp, Artificial Sequence
  Primer
acgttggatgcttctgtgatgtcacactcc

SEQ ID NO: 59 - DNA, 30 bp, Artificial Sequence
  Primer
acgttggatggagaaaagcctgcagggcta

SEQ ID NO: 60 - DNA, 29 bp, Artificial Sequence
  Primer
acgttggatggtgctgttctcccagcaga

SEQ ID NO: 61 - DNA, 29 bp, Artificial Sequence
  Primer
acgttggatggattccagagtccaggaac

SEQ ID NO: 62 - DNA, 30 bp, Artificial Sequence
  Primer
acgttggatgattttgcagaggagtgggac

SEQ ID NO: 63 - DNA, 30 bp, Artificial Sequence
  Primer
acgttggatgatcattctggggtaataggc

SEQ ID NO: 64 - DNA, 30 bp, Artificial Sequence
  Primer
acgttggatgtctctctccactcaatctcg

SEQ ID NO: 65 - DNA, 30 bp, Artificial Sequence
  Primer
acgttggatgtgttgggaaggtttagatgc

SEQ ID NO: 66 - DNA, 30 bp, Artificial Sequence
  Primer
acgttggatgaacaaggtagcccagccaag

SEQ ID NO: 67 - DNA, 30 bp, Artificial Sequence
  Primer
acgttggatgaggaccataacctatgggac

SEQ ID NO: 68 - DNA, 30 bp, Artificial Sequence
  Primer
acgttggatgtcagaatcagtcacttaagg

SEQ ID NO: 69 - DNA, 31 bp, Artificial Sequence
  Primer
acgttggatggaatgaagtattggtgtgtgc

SEQ ID NO: 70 - DNA, 30 bp, Artificial Sequence
  Primer
acgttggatgccttttggaatgtgacctac

SEQ ID NO: 71 - DNA, 30 bp, Artificial Sequence
  Primer
acgttggatgcccatttgagaactctgacc

SEQ ID NO: 72 - DNA, 30 bp, Artificial Sequence
  Primer
acgttggatggtttcttgttgctgctataa

SEQ ID NO: 73 - DNA, 30 bp, Artificial Sequence
  Primer
acgttggatggttcctagtctaaaatagtc

SEQ ID NO: 74 - DNA, 30 bp, Artificial Sequence
  Primer
acgttggatggaaggtacagaagacatgag

SEQ ID NO: 75 - DNA, 30 bp, Artificial Sequence
  Primer
acgttggatgatgtgtggcttgcaagaacc

SEQ ID NO: 76 - DNA, 30 bp, Artificial Sequence
  Primer
acgttggatgaccttggactttcttggcac

SEQ ID NO: 77 - DNA, 30 bp, Artificial Sequence
  Primer
acgttggatgtttcctaccccaaacacatc

SEQ ID NO: 78 - DNA, 30 bp, Artificial Sequence
  Primer
acgttggatggctgattcatgagtactctc

SEQ ID NO: 79 - DNA, 30 bp, Artificial Sequence
  Primer
acgttggatgagcagaaggcaacacgtttc

SEQ ID NO: 80 - DNA, 30 bp, Artificial Sequence
  Primer
acgttggatgcctgctgcttttgggttttt

SEQ ID NO: 81 - DNA, 29 bp, Artificial Sequence
  Primer
acgttggatgatgagttggaggagggagc

SEQ ID NO: 82 - DNA, 30 bp, Artificial Sequence
  Primer
acgttggatgctttggcggtgagtttttac

SEQ ID NO: 83 - DNA, 30 bp, Artificial Sequence
  Primer
acgttggatggctgctccagaaaatgacag

SEQ ID NO: 84 - DNA, 30 bp, Artificial Sequence
  Primer
acgttggatgtctccgtcttcttgatcacc

SEQ ID NO: 85 - DNA, 30 bp, Artificial Sequence
  Primer
acgttggatggtgtcctagatagagaccag

SEQ ID NO: 86 - DNA, 30 bp, Artificial Sequence
  Primer
acgttggatggatcagatatttatgggagg

SEQ ID NO: 87 - DNA, 30 bp, Artificial Sequence
  Primer
acgttggatgaaagttgcctcagatactcc

SEQ ID NO: 88 - DNA, 30 bp, Artificial Sequence
  Primer
acgttggatgcaggtaggtgagttccatac

SEQ ID NO: 89 - DNA, 30 bp, Artificial Sequence
  Primer
acgttggatgtcaacttggtgaccaagaag

SEQ ID NO: 90 - DNA, 30 bp, Artificial Sequence
  Primer
acgttggatgcaggatgtccatgttggaac

SEQ ID NO: 91 - DNA, 30 bp, Artificial Sequence
  Primer
acgttggatgaccaattacaaagggcagat

SEQ ID NO: 92 - DNA, 30 bp, Artificial Sequence
  Primer
acgttggatggtgggttgcttgtggataac

SEQ ID NO: 93 - DNA, 30 bp, Artificial Sequence
  Primer
acgttggatgtaggaggggttaatagagag

SEQ ID NO: 94 - DNA, 31 bp, Artificial Sequence
  Primer
acgttggatgggttagtatttattgagattg

SEQ ID NO: 95 - DNA, 22 bp, Artificial Sequence
  Primer
tgatttttaaatctgttgacaa

SEQ ID NO: 96 - DNA, 22 bp, Artificial Sequence
  Primer
ggcacctccagctttagttttc

SEQ ID NO: 97 - DNA, 19 bp, Artificial Sequence
  Primer
aaaagcagctgggccaaga

SEQ ID NO: 98 - DNA, 19 bp, Artificial Sequence
  Primer
ccatcaagtcatttcctct

SEQ ID NO: 99 - DNA, 22 bp, Artificial Sequence
  Primer
ccacccataccacagattgtac

SEQ ID NO: 100 - DNA, 26 bp, Artificial Sequence
  Primer
tgagatatataaatggccaagaaata

SEQ ID NO: 101 - DNA, 17 bp, Artificial Sequence
  Primer
tatcaacagcagccagc

SEQ ID NO: 102 - DNA, 21 bp, Artificial Sequence
  Primer
gcctcggcgtcaaggatgggg

SEQ ID NO: 103 - DNA, 19 bp, Artificial Sequence
  Primer
caaccccatactaaactcc

SEQ ID NO: 104 - DNA, 18 bp, Artificial Sequence
  Primer
tctctgctataccaacac

SEQ ID NO: 105 - DNA, 26 bp, Artificial Sequence
  Primer
aaactctgggagattctcctattgac

SEQ ID NO: 106 - DNA, 17 bp, Artificial Sequence
  Primer
tctaggcccctcacccc

SEQ ID NO: 107 - DNA, 19 bp, Artificial Sequence
  Primer
caaggtgtggaaaggtata

SEQ ID NO: 108 - DNA, 20 bp, Artificial Sequence
  Primer
tgtcttccagggaaaaaggg

SEQ ID NO: 109 - DNA, 17 bp, Artificial Sequence
  Primer
aggcagcacagactgga

SEQ ID NO: 110 - DNA, 17 bp, Artificial Sequence
  Primer
ggcacggtacctgggct

SEQ ID NO: 111 - DNA, 21 bp, Artificial Sequence
  Primer
tctatgttgtcttgttttgag

SEQ ID NO: 112 - DNA, 26 bp, Artificial Sequence
  Primer
ggagggacattactttaaaagtgcaa

SEQ ID NO: 113 - DNA, 23 bp, Artificial Sequence
  Primer
ctatacctgtaccccgggttttg

SEQ ID NO: 114 - DNA, 26 bp, Artificial Sequence
  Primer
ctcatcgcatacattttggggagcta

SEQ ID NO: 115 - DNA, 26 bp, Artificial Sequence
  Primer
ctgggcccagacagtttctttgggaa

SEQ ID NO: 116 - DNA, 23 bp, Artificial Sequence
  Primer
ggttttagactcagtttcaggta

SEQ ID NO: 117 - DNA, 17 bp, Artificial Sequence
  Primer
gactgacccactgcttc

SEQ ID NO: 118 - DNA, 21 bp, Artificial Sequence
  Primer
ccccccctgtattttagtttt

SEQ ID NO: 119 - DNA, 22 bp, Artificial Sequence
  Primer
ggttagaccatgactgacacat

SEQ ID NO: 120 - DNA, 21 bp, Artificial Sequence
  Primer
gagctaagcactttttagata

SEQ ID NO: 121 - DNA, 23 bp, Artificial Sequence
  Primer
tgccagttgcccactgtggcaat

SEQ ID NO: 122 - DNA, 25 bp, Artificial Sequence
  Primer
actcttgacaacaagataggcttta

SEQ ID NO: 123 - DNA, 21 bp, Artificial Sequence
  Primer
atcctttcttcccacctgacc

SEQ ID NO: 124 - DNA, 25 bp, Artificial Sequence
  Primer
tatgttctcccagcagagaacactc

SEQ ID NO: 125 - DNA, 17 bp, Artificial Sequence
  Primer
ggagtgggacaaaacta

SEQ ID NO: 126 - DNA, 18 bp, Artificial Sequence
  Primer
tgccagccaatgttctga

SEQ ID NO: 127 - DNA, 24 bp, Artificial Sequence
  Primer
gcccagccaagcatggctccaagg

SEQ ID NO: 128 - DNA, 25 bp, Artificial Sequence
  Primer
aaagcagtcacttaaggaagaagag

SEQ ID NO: 129 - DNA, 23 bp, Artificial Sequence
  Primer
gagggaatgtgacctacactatg

SEQ ID NO: 130 - DNA, 26 bp, Artificial Sequence
  Primer
taatagctaacataatacaaagttac

SEQ ID NO: 131 - DNA, 23 bp, Artificial Sequence
  Primer
acatgagactttcatgaattata

SEQ ID NO: 132 - DNA, 26 bp, Artificial Sequence
  Primer
ggcacgctcatgttttgtttttgaag

SEQ ID NO: 133 - DNA, 24 bp, Artificial Sequence
  Primer
tcatgagtactctcacattaacaa

SEQ ID NO: 134 - DNA, 24 bp, Artificial Sequence
  Primer
ggacactagtaaaaaactactcag

SEQ ID NO: 135 - DNA, 17 bp, Artificial Sequence
  Primer
gagtcgcagcatggcct

SEQ ID NO: 136 - DNA, 21 bp, Artificial Sequence
  Primer
gctcacctgcagtagctgcac

SEQ ID NO: 137 - DNA, 17 bp, Artificial Sequence
  Primer
ttatgggaggtatgcag

SEQ ID NO: 138 - DNA, 19 bp, Artificial Sequence
  Primer
caccagtgttttttactga

SEQ ID NO: 139 - DNA, 19 bp, Artificial Sequence
  Primer
tgctgcagccagtttctcc

SEQ ID NO: 140 - DNA, 19 bp, Artificial Sequence
  Primer
ttttttatccgctctgaca

SEQ ID NO: 141 - DNA, 24 bp, Artificial Sequence
  Primer
ttgagattgttagatctatgtatt

SEQ ID NO: 142 - DNA, 51 bp, Homo sapiens
  variation 26: n = a or g
gtctgatttttaaatctgttgacaanagaaggctgaaactggaacataaga

SEQ ID NO: 143 - DNA, 51 bp, Homo sapiens
  variation 26: n = c or t
cagctcacctccagctttagttttcncatgacagtaagtctattaccctcc

SEQ ID NO: 144 - DNA, 51 bp, Homo sapiens
  variation 26: n = c or t
agtgcttgtgtccctggtctctgggntcttggcccagctgcttttctgatt

SEQ ID NO: 145 - DNA, 51 bp, Homo sapiens
  variation 26: n = a or g
tccgtaccatcaagtcatttcctctngacgtctgaacctgcactcagggtc

SEQ ID NO: 146 - DNA, 51 bp, Homo sapiens
  variation 26: n = a or c
tgggaacagggcaatgcattttaccngtacaatctgtggtatgggtggtgt

SEQ ID NO: 147 - DNA, 51 bp, Homo sapiens
  variation 26: n = a or g
cccggacaatgttgggaattttttcntatttcttggccatttatatatctt

SEQ ID NO: 148 - DNA, 51 bp, Homo sapiens
  variation 26: n = c or t
tgcttctttatcaacagcagccagcngggacagccaagtggttcggagaga

SEQ ID NO: 149 - DNA, 51 bp, Homo sapiens
  variation 26: n = g or t
ggggagcccactggggtcagaggctnccccatccttgacgccgaggcccct

SEQ ID NO: 150 - DNA, 51 bp, Homo sapiens
  variation 26: n = a or c
cctcagtttacccagttctcaactgnggagtttagtatggggttaaaatgg

SEQ ID NO: 151 - DNA, 51 bp, Homo sapiens
  variation 26: n = c or t
actggtctctctgctataccaacacntttggctcagcctcagctccagcca

SEQ ID NO: 152 - DNA, 51 bp, Homo sapiens
  variation 26: n = c or g
aactctgggagattctcctattgacncagaaagcgattccttcactgatac

SEQ ID NO: 153 - DNA, 51 bp, Homo sapiens
  variation 26: n = c or t
ggagctgtcacaggactttgccaccnggggtgaggggcctagaaacccctc

SEQ ID NO: 154 - DNA, 51 bp, Homo sapiens
  variation 26: n = c or t
aaataaactggtaggagtaagggctntatacctttccacaccttaaaacca

SEQ ID NO: 155 - DNA, 51 bp, Homo sapiens
  variation 26: n = a or g
acctgtgtcttccagggaaaaagggnctcagggctcagcccaggcagggaa

SEQ ID NO: 156 - DNA, 51 bp, Homo sapiens
  variation 26: n = a or g
atacagagaggcagcacagactgganatgctgcataaagcttaaattgggc

SEQ ID NO: 157 - DNA, 51 bp, Homo sapiens
  variation 26: n = c or t
cgctggcgggcacggtacctgggctnggcagggtcctctgccaggcgtgtc

SEQ ID NO: 158 - DNA, 51 bp, Homo sapiens
  variation 26: n = g or t
aaggcaaaggatctgaatagctattnctcaaaacaagacaacatagaaatg

SEQ ID NO: 159 - DNA, 51 bp, Homo sapiens
  variation 26: n = a or g
cttggacattactttaaaagtgcaantgacaaaagaaaaatatagataaat

SEQ ID NO: 160 - DNA, 51 bp, Homo sapiens
  variation 26: n = c or g
cccgatacctgtaccccgggttttgngctgacacatgctccattgcttcct

SEQ ID NO: 161 - DNA, 51 bp, Homo sapiens
  variation 26: n = a or g
agaaggcccctccttcccttgaccantagctccccaaaatgtatgcgatga

SEQ ID NO: 162 - DNA, 51 bp, Homo sapiens
  variation 26: n = a or g
tgtcactgtctacaaaattgttaatnttcccaaagaaactgtctgggcccc

SEQ ID NO: 163 - DNA, 51 bp, Homo sapiens
  variation 26: n = a or c
atacagggtcttcatttatttttagntacctgaaactgagtctaaaaccac

SEQ ID NO: 164 - DNA, 51 bp, Homo sapiens
  variation 26: n = a or g
gtgaattctttaagaaaatgaagccngaagcagtgggtcagtctgtaatcc

SEQ ID NO: 165 - DNA, 51 bp, Homo sapiens
  variation 26: n = a or g
atattcccccctgtattttagttttngatctacagttatgtagcaatgagc

SEQ ID NO: 166 - DNA, 51 bp, Homo sapiens
  variation 26: n = a or g
gttttttagaccatgactgacacatntttgctgatcaatacatttgttgag

SEQ ID NO: 167 - DNA, 51 bp, Homo sapiens
  variation 26: n = c or t
tagagagctaagcactttttagatantatataatttaattgccgtatgagg

SEQ ID NO: 168 - DNA, 51 bp, Homo sapiens
  variation 26: n = a or c
catgccagttgcccactgtggcaatnaatatctgagcctgtggtttttgcc

SEQ ID NO: 169 - DNA, 51 bp, Homo sapiens
  variation 26: n = c or t
cctcttgacaacaagataggctttanttcgcctctagaatggcctccagcc

SEQ ID NO: 170 - DNA, 51 bp, Homo sapiens
  variation 26: n = c or t
atgaaggattccaaggagcagcagtnggtcaggtgggaagaaaggagtgtg

SEQ ID NO: 171 - DNA, 51 bp, Homo sapiens
  variation 26: n = c or t
gctgttctcccagcagagaacactcngtttcctgcccaccctctacccctc

SEQ ID NO: 172 - DNA, 51 bp, Homo sapiens
  variation 26: n = c or t
gctatcaactaatgctgtttttcacntagttttgtcccactcctctgcaaa

SEQ ID NO: 173 - DNA, 51 bp, Homo sapiens
  variation 26: n = g or t
tgtttcatgccagccaatgttctgangttccagcatgcttcacagcaaagc

SEQ ID NO: 174 - DNA, 51 bp, Homo sapiens
  variation 26: n = g or t
agcccagccaagcatggctccaaggntgaagagagctgcaaggaggcaagg

SEQ ID NO: 175 - DNA, 51 bp, Homo sapiens
  variation 26: n = a or g
cttttcttttggctttaaattatgcnctcttcttccttaagtgactgattc

SEQ ID NO: 176 - DNA, 51 bp, Homo sapiens
  variation 26: n = a or g
cttttggaatgtgacctacactatgnaacccagcacgtacacacacaggca

SEQ ID NO: 177 - DNA, 51 bp, Homo sapiens
  variation 26: n = c or t
aatagctaacataatacaaagttacnagcttgcacttatgtaaggtcagag

SEQ ID NO: 178 - DNA, 51 bp, Homo sapiens
  variation 26: n = a or g
agacatgagactttcatgaattatancatgactccttaaataatacaaaag

SEQ ID NO: 179 - DNA, 51 bp, Homo sapiens
  variation 26: n = c or g
gcacgctcatgttttgtttttgaagngcaggatctgagccggttcttgcaa

SEQ ID NO: 180 - DNA, 51 bp, Homo sapiens
  variation 26: n = a or g
atgacaattcagtgtgtggtggaatnttgttaatgtgagagtactcatgaa

SEQ ID NO: 181 - DNA, 51 bp, Homo sapiens
  variation 26: n = a or t
aaagcatttccgcgcttggaagaagnctgagtagttttttactagtgtaaa

SEQ ID NO: 182 - DNA, 51 bp, Homo sapiens
  variation 26: n = c or g
tgagttggaggagggagccccttccnaggccatgctgcgactcctgcaaag

SEQ ID NO: 183 - DNA, 51 bp, Homo sapiens
  variation 26: n = c or t
acctcagacagattgtcacggaacangtgcagctactgcaggtgatcaaga

SEQ ID NO: 184 - DNA, 51 bp, Homo sapiens
  variation 26: n = g or t
tcagatatttatgggaggtatgcagnttttaaattctgagaatttgtactg

SEQ ID NO: 185 - DNA, 51 bp, Homo sapiens
  variation 26: n = c or t
atactccaatctgacctgatttgacntcagtaaaaaacactggtgaagtag

SEQ ID NO: 186 - DNA, 51 bp, Homo sapiens
  variation 26: n = a or g
gtttctgagctttgtggcctaccaanggagaaactggctgcagcagagcga

SEQ ID NO: 187 - DNA, 51 bp, Homo sapiens
  variation 26: n = a or c
ccaattacaaagggcagatcagagantgtcagagcggataaaaaatcaaga

SEQ ID NO: 188 - DNA, 51 bp, Homo sapiens
  variation 26: n = a or c
tttcttcaaatttgatgaaaacttcnaatacatagatctaacaatctcaat

(19) United States
(12) Patent Application Publication
(10) Pub. No.: US 2017/0009299 A1
(43) Pub. Date: Jan. 12, 2017

(54) COMPOSITIONS AND METHODS FOR DETECTING SNP(S) ASSOCIATED WITH DIABETES

(71) Applicants: CapitalBio eHealth Science & Technology (Beijing) Co., Ltd., Beijing (CN); CapitalBio Corporation, Beijing (CN); Tsinghua University, Beijing (CN); Shanghai Sixth People's Hospital, Shanghai (CN)

(72) Inventors: Lan XIE, Beijing (CN); Weiping JIA, Beijing (CN); Yimin SUN, Beijing (CN); Cheng HU, Beijing (CN); Lin SHAO, Beijing (CN); Jing CHENG, Beijing (CN)

(73) Assignees: CapitalBio eHealth Science & Technology (Beijing) Co., Ltd., Beijing (CN); CapitalBio Corporation, Beijing (CN); Tsinghua University, Beijing (CN); Shanghai Sixth People's Hospital, Shanghai (CN)

(21) Appl. No.: 15/203,708

(22) Filed: Jul. 6, 2016

(30) Foreign Application Priority Data
Jul. 7, 2015 (CN) 201510393858.3

(51) Int. Cl. C12Q 1/68; A61K 31/625

(52) U.S. Cl. CPC C12Q 1/6883; A61K 31/625; C12Q 2600/106; C12Q 2600/156; C12Q 2600/158; C12Q 2600/16; C12Q 2600/118

(57) ABSTRACT

In one aspect, provided herein are set of primers and use of the same for the detection of SNPs associated with diabetes. In certain embodiments, the primers used to detect SNP sites associated with diabetes comprise Primer Set 1 to Primer Set 47. The experiments show: the genotyping results of the SNP sites associated with diabetes can be accurately detected by the primers disclosed herein, and the risk of individuals can be comprehensively evaluated and the result is more accurate than the single site analysis. In addition, SNPs disclosed herein are verified as associated with type 2 diabetes and its complications, which are especially suitable for the prevention and individualized treatment for type 2 diabetes in East Asian, for example, in China.

### CROSS-REFERENCE TO RELATED APPLICATIONS

This application claims priority to Chinese Patent Application No. 201510393858.3, filed on Jul. 7, 2015, published on Dec. 2, 2015 as CN 105112502 A, the content of which is incorporated by reference herein in its entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 768332000100SeqList.txt, date recorded: 6 Jul. 2016, size: 33,157 bytes).

### TECHNICAL FIELD

The present disclosure relates to the field of biotechnology, and specifically, compositions and methods for the risk assessment, diagnosis, and/or prognosis of diabetes or a related disease or condition, for example, via detection of SNP (Single Nucleotide Polymorphism) or SNPs associated with diabetes or a related disease or condition. In particular aspects, the present disclosure relates to a primer or a primer set, and a reagent, composition, or kit comprising the same, as well as a method of use, for detecting the SNP(s). In particular aspects, the primers, primer sets, reagents, compositions, kits, and methods are for detection of SNP(s) associated with type 2 diabetes mellitus (e.g., in an East Asian population), diabetic nephropathy, diabetic retinitis (DR), diabetic cardiopathy (such as diabetic cardiomyopathy or elderly diabetic cardiopathy), and/or drug sensitivity (such as drug sensitivity to a diabetes medication).

### BACKGROUND

Diabetes is a group of chronic metabolic diseases caused by defect in insulin secretion and/or disorders of insulin action, characterized by high blood sugar. Long-term sustained hyperglycemia and metabolic disorders can lead to the damage of multiple organs, particularly to the eyes, kidneys, cardiovascular and nervous systems, eventually resulting in serious consequences, such us blindness, stroke, myocardial infarction, amputation and renal failure.

Diabetes is prevalent in China. According to the data in 2010, the prevalence rate of diabetes is 9.7% in adults more than 18 years, and the number of patients with diabetes has reached 97 million. International Diabetes Federation (IDF) believes that if the trend continues, in 2035 Chinese patients with diabetes will reach 143 million. Diabetes and its complications have not only seriously affected the life quality of patients, but also led to the rapid rise in health care costs. From 1993 to 2007, the health care costs of diabetes rose from 200 to 221.6 billion yuan, the ratio of direct medical costs of diabetes to the total health expenditure rose from 1.96 to 18.2 percent. Loss of life and economic burden caused by diabetes has been overwhelming.

Diabetes is the result of the interaction of genetic and environmental factors. On the one hand, improvement of living conditions and increased incidence of obesity are important factors in increasing the incidence of type 2 diabetes. On the other hand, genetic factors also play an indispensable role. The study of Newman et al. in twins suggests that genetic factors play roles in type 2 diabetes. See Newman et al., 1987, Diabetologia 30(10):763-8. Diabetes and complications are preventable and controllable by predicting susceptibility to diabetes and complications, and early health intervention is an effective way to prevent diabetes and its progression. Furthermore, it is worth noting that due to the differences in genetic background and so on, the sensitivity of individuals to even the same kind of diabetes treatment may be different. Therefore, there is a need for personalized medicine through genetic testing and other means, which is the key to control diabetes progression. The present disclosure addresses this and the related needs.

### SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

SNP is the DNA sequence polymorphism caused by a single nucleotide variation on the genomic level. SNP appears at every 500-1000 base pairs on the human genome and is the most common genetic variation and also excellent molecular markers in polygenic disease researches. In recent years, benefited from the development of high-throughput genome sequencing, many SNP sites have been discovered which are associated with diabetes, diabetic complications and anti-diabetic drug sensitivity. By detecting these sites, the risk of developing diabetes and complications can be predicted, and the sensitivity to various drugs can be evaluated, which is helpful for diabetes treatment. However, there are racial and regional differences in the presence of SNPs and allele frequency, the diabetes-related SNPs reported in Western countries or group of Caucasian may be different form that in the Chinese people. Diabetes is a complex and polygenic disease where multiple SNPs are involved. Therefore, to take advantage of SNP to predict the development trend of diabetes and susceptibility, in some aspects, it is necessary to consider the combination of SNPs and the differences between the ethnic and geography. Currently, there are no methods or products for the detection of diabetes-related SNP combinations, especially for East Asians.

Diabetes prevalence is a major health problem in China now and will continue to be so in at least the near future. It is important to predict individual risk for diabetes and complications, to treat the disease before its onset, and to administrate personalized medicine. Thus, it is urgent to develop the methods and products for the detection of the SNP sites associated with type 2 diabetes mellitus in East Asian, susceptibility to complications and anti-diabetes drugs sensitivity.

In one aspect, disclosed herein is an isolated polynucleotide comprising a single nucleotide polymorphism (SNP) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342; a complementary sequence thereof; and/or sequences in linkage disequilibrium therewith. In specific embodiments, the isolated polynucleotide comprises a nucleic acid sequence selected from the group consisting of SEQ ID NOs: 142-188.

In another aspect, disclosed herein is a panel of isolated polynucleotides, the polynucleotides comprising two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, or all 47 of the single nucleotide polymorphisms (SNPs) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, or complementary sequences thereof, and/or sequences in linkage disequilibrium therewith.

In another aspect, disclosed herein is a panel of isolated biomarkers comprising two or more SNPs selected from the group consisting of rs10229583, rs10811661, rs10886471, rs111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941 rs245955, rs245962, rs266729, rs3811951, rs156019, rs6234, rs3856806, rs10494366, rs11977021, rs2230806, rs11212617, and rs622342, or complementary sequences thereof, and/or sequences in linkage disequilibrium therewith.

In yet another aspect, disclosed herein is a panel of isolated biomarkers associated and/or linked with two or more of the SNPs selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, or complementary sequences thereof, and/or sequences in linkage disequilibrium therewith.

In any of the preceding embodiments, the one or more SNPs can be associated with diabetes mellitus and/or a disease or condition related to diabetes mellitus, such as type 2 diabetes mellitus, diabetic nephropathy, diabetic retinitis, diabetic cardiomyopathy (e.g., elderly diabetic cardiomyopathy), and/or drug resistance to an anti-diabetes medication.

In one aspect, disclosed herein is an isolated polynucleotide or a set of isolated polynucleotides for detecting one or more of the SNPs selected from the group consisting of rs10229583, rs10811661, rs10886471, rs111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342; and/or complementary sequences thereof; and/or sequences in linkage disequilibrium therewith.

In one aspect, disclosed herein is an isolated polynucleotide comprising a nucleic acid sequence having at least about 85%, at least about 900/%, at least about 95%, at least about 99%, or 100% sequence homology or identity with any of SEQ ID NOs: 1-141.

In another aspect, provided herein is a set of isolated polynucleotides comprising nucleic acid sequences having at least about 85%, at least about 90%, at least about 95%, at least about 99%, or 100% sequence homology or identity with one or more of SEQ ID NOs: 1-141.

In one aspect, disclosed herein is an isolated polynucleotide comprising the nucleic acid sequence set forth in any of SEQ ID NOs: 1-141. In another aspect, disclosed herein is a set of isolated polynucleotides comprising one or more, two or more, or three or more of the nucleic acid sequences set forth in SEQ ID NOs: 1-141. In one embodiment, the set of isolated polynucleotides comprises one or more, or two or more, of the nucleic acid sequences set forth in SEQ ID NOs: 1-94. In another embodiment, the set of isolated polynucleotides comprises one or more of the nucleic acid sequences set forth in SEQ ID NOs: 95-141.

In any of the preceding embodiments, the isolated polynucleotide and/or the set of isolated polynucleotides are for detecting one or more of the SNPs selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342; and/or complementary sequences thereof; and/or sequences in linkage disequilibrium therewith. In one embodiment, the set of isolated polynucleotides comprises at least two amplification primers for detecting the one or more SNPs. In another embodiment, the set of isolated polynucleotides comprises at least one primer for single base extension for detecting the one or more SNPs. In yet another embodiment, the set of isolated polynucleotides comprises at least two amplification primers and at least one primer for single base extension for detecting the one or more SNPs.

In any of the preceding embodiments, the set of isolated polynucleotides can comprise two amplification primers and/or a primer for single base extension, for detecting any one or more of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342.

In any of the preceding embodiments, the set of isolated polynucleotides can comprise two amplification primers which comprise nucleic acid sequences set forth in SEQ ID NOs: 1 and 2, respectively, and/or a primer for single base extension which comprises the nucleic acid sequence set forth in SEQ ID NO: 95.

In any of the preceding embodiments, the set of isolated polynucleotides can comprise two amplification primers which comprise nucleic acid sequences set forth in SEQ ID NOs: 3 and 4, respectively, and/or a primer for single base extension which comprises the nucleic acid sequence set forth in SEQ ID NO: 96.

In any of the preceding embodiments, the set of isolated polynucleotides can comprise two amplification primers which comprise nucleic acid sequences set forth in SEQ ID NOs: 5 and 6, respectively, and/or a primer for single base extension which comprises the nucleic acid sequence set forth in SEQ ID NO: 97.

In any of the preceding embodiments, the set of isolated polynucleotides can comprise two amplification primers which comprise nucleic acid sequences set forth in SEQ ID NOs: 7 and 8, respectively, and/or a primer for single base extension which comprises the nucleic acid sequence set forth in SEQ ID NO: 98.

In any of the preceding embodiments, the set of isolated polynucleotides can comprise two amplification primers which comprise nucleic acid sequences set forth in SEQ ID NOs: 9 and 10, respectively, and/or a primer for single base extension which comprises the nucleic acid sequence set forth in SEQ ID NO: 99.

In any of the preceding embodiments, the set of isolated polynucleotides can comprise two amplification primers which comprise nucleic acid sequences set forth in SEQ ID NOs: 11 and 12, respectively, and/or a primer for single base extension which comprises the nucleic acid sequence set forth in SEQ ID NO: 100.

In any of the preceding embodiments, the set of isolated polynucleotides can comprise two amplification primers which comprise nucleic acid sequences set forth in SEQ ID NOs: 13 and 14, respectively, and/or a primer for single base extension which comprises the nucleic acid sequence set forth in SEQ ID NO: 101.

In any of the preceding embodiments, the set of isolated polynucleotides can comprise two amplification primers which comprise nucleic acid sequences set forth in SEQ ID NOs: 15 and 16, respectively, and/or a primer for single base extension which comprises the nucleic acid sequence set forth in SEQ ID NO: 102.

In any of the preceding embodiments, the set of isolated polynucleotides can comprise two amplification primers which comprise nucleic acid sequences set forth in SEQ ID NOs: 17 and 18, respectively, and/or a primer for single base extension which comprises the nucleic acid sequence set forth in SEQ ID NO: 103.

In any of the preceding embodiments, the set of isolated polynucleotides can comprise two amplification primers which comprise nucleic acid sequences set forth in SEQ ID NOs: 19 and 20, respectively, and/or a primer for single base extension which comprises the nucleic acid sequence set forth in SEQ ID NO: 104.

In any of the preceding embodiments, the set of isolated polynucleotides can comprise two amplification primers which comprise nucleic acid sequences set forth in SEQ ID NOs: 21 and 22, respectively, and/or a primer for single base extension which comprises the nucleic acid sequence set forth in SEQ ID NO: 105.

In any of the preceding embodiments, the set of isolated polynucleotides can comprise two amplification primers which comprise nucleic acid sequences set forth in SEQ ID NOs: 23 and 24, respectively, and/or a primer for single base extension which comprises the nucleic acid sequence set forth in SEQ ID NO: 106.

In any of the preceding embodiments, the set of isolated polynucleotides can comprise two amplification primers which comprise nucleic acid sequences set forth in SEQ ID NOs: 25 and 26, respectively, and/or a primer for single base extension which comprises the nucleic acid sequence set forth in SEQ ID NO: 107.

In any of the preceding embodiments, the set of isolated polynucleotides can comprise two amplification primers which comprise nucleic acid sequences set forth in SEQ ID NOs: 27 and 28, respectively, and/or a primer for single base extension which comprises the nucleic acid sequence set forth in SEQ ID NO: 108.

In any of the preceding embodiments, the set of isolated polynucleotides can comprise two amplification primers which comprise nucleic acid sequences set forth in SEQ ID NOs: 29 and 30, respectively, and/or a primer for single base extension which comprises the nucleic acid sequence set forth in SEQ ID NO: 109.

In any of the preceding embodiments, the set of isolated polynucleotides can comprise two amplification primers which comprise nucleic acid sequences set forth in SEQ ID NOs: 31 and 32, respectively, and/or a primer for single base extension which comprises the nucleic acid sequence set forth in SEQ ID NO: 110.

In any of the preceding embodiments, the set of isolated polynucleotides can comprise two amplification primers which comprise nucleic acid sequences set forth in SEQ ID NOs: 33 and 34, respectively, and/or a primer for single base extension which comprises the nucleic acid sequence set forth in SEQ ID NO: 111.

In any of the preceding embodiments, the set of isolated polynucleotides can comprise two amplification primers which comprise nucleic acid sequences set forth in SEQ ID NOs: 35 and 36, respectively, and/or a primer for single base extension which comprises the nucleic acid sequence set forth in SEQ ID NO: 112.

In any of the preceding embodiments, the set of isolated polynucleotides can comprise two amplification primers which comprise nucleic acid sequences set forth in SEQ ID NOs: 37 and 38, respectively, and/or a primer for single base extension which comprises the nucleic acid sequence set forth in SEQ ID NO: 113.

In any of the preceding embodiments, the set of isolated polynucleotides can comprise two amplification primers which comprise nucleic acid sequences set forth in SEQ ID NOs: 39 and 40, respectively, and/or a primer for single base extension which comprises the nucleic acid sequence set forth in SEQ ID NO: 114.

In any of the preceding embodiments, the set of isolated polynucleotides can comprise two amplification primers which comprise nucleic acid sequences set forth in SEQ ID NOs: 41 and 42, respectively, and/or a primer for single base extension which comprises the nucleic acid sequence set forth in SEQ ID NO: 115.

In any of the preceding embodiments, the set of isolated polynucleotides can comprise two amplification primers which comprise nucleic acid sequences set forth in SEQ ID NOs: 43 and 44, respectively, and/or a primer for single base extension which comprises the nucleic acid sequence set forth in SEQ ID NO: 116.

In any of the preceding embodiments, the set of isolated polynucleotides can comprise two amplification primers which comprise nucleic acid sequences set forth in SEQ ID NOs: 45 and 46, respectively, and/or a primer for single base extension which comprises the nucleic acid sequence set forth in SEQ ID NO: 117.

In any of the preceding embodiments, the set of isolated polynucleotides can comprise two amplification primers which comprise nucleic acid sequences set forth in SEQ ID NOs: 47 and 48, respectively, and/or a primer for single base extension which comprises the nucleic acid sequence set forth in SEQ ID NO: 118.

In any of the preceding embodiments, the set of isolated polynucleotides can comprise two amplification primers which comprise nucleic acid sequences set forth in SEQ ID NOs: 49 and 50, respectively, and/or a primer for single base extension which comprises the nucleic acid sequence set forth in SEQ ID NO: 119.

In any of the preceding embodiments, the set of isolated polynucleotides can comprise two amplification primers which comprise nucleic acid sequences set forth in SEQ ID NOs: 51 and 52, respectively, and/or a primer for single base extension which comprises the nucleic acid sequence set forth in SEQ ID NO: 120.

In any of the preceding embodiments, the set of isolated polynucleotides can comprise two amplification primers which comprise nucleic acid sequences set forth in SEQ ID NOs: 53 and 54, respectively, and/or a primer for single base extension which comprises the nucleic acid sequence set forth in SEQ ID NO: 121.

In any of the preceding embodiments, the set of isolated polynucleotides can comprise two amplification primers which comprise nucleic acid sequences set forth in SEQ ID NOs: 55 and 56, respectively, and/or a primer for single base extension which comprises the nucleic acid sequence set forth in SEQ ID NO: 122.

In any of the preceding embodiments, the set of isolated polynucleotides can comprise two amplification primers which comprise nucleic acid sequences set forth in SEQ ID NOs: 57 and 58, respectively, and/or a primer for single base extension which comprises the nucleic acid sequence set forth in SEQ ID NO: 123.

In any of the preceding embodiments, the set of isolated polynucleotides can comprise two amplification primers which comprise nucleic acid sequences set forth in SEQ ID NOs: 59 and 60, respectively, and/or a primer for single base extension which comprises the nucleic acid sequence set forth in SEQ ID NO: 124.

In any of the preceding embodiments, the set of isolated polynucleotides can comprise two amplification primers which comprise nucleic acid sequences set forth in SEQ ID NOs: 61 and 62, respectively, and/or a primer for single base extension which comprises the nucleic acid sequence set forth in SEQ ID NO: 125.

In any of the preceding embodiments, the set of isolated polynucleotides can comprise two amplification primers which comprise nucleic acid sequences set forth in SEQ ID NOs: 63 and 64, respectively, and/or a primer for single base extension which comprises the nucleic acid sequence set forth in SEQ ID NO: 126.

In any of the preceding embodiments, the set of isolated polynucleotides can comprise two amplification primers which comprise nucleic acid sequences set forth in SEQ ID NOs: 65 and 66, respectively, and/or a primer for single base extension which comprises the nucleic acid sequence set forth in SEQ ID NO: 127.

In any of the preceding embodiments, the set of isolated polynucleotides can comprise two amplification primers which comprise nucleic acid sequences set forth in SEQ ID NOs: 67 and 68, respectively, and/or a primer for single base extension which comprises the nucleic acid sequence set forth in SEQ ID NO: 128.

In any of the preceding embodiments, the set of isolated polynucleotides can comprise two amplification primers which comprise nucleic acid sequences set forth in SEQ ID NOs: 69 and 70, respectively, and/or a primer for single base extension which comprises the nucleic acid sequence set forth in SEQ ID NO: 129.

In any of the preceding embodiments, the set of isolated polynucleotides can comprise two amplification primers which comprise nucleic acid sequences set forth in SEQ ID NOs: 71 and 72, respectively, and/or a primer for single base extension which comprises the nucleic acid sequence set forth in SEQ ID NO: 130.

In any of the preceding embodiments, the set of isolated polynucleotides can comprise two amplification primers which comprise nucleic acid sequences set forth in SEQ ID NOs: 73 and 74, respectively, and/or a primer for single base extension which comprises the nucleic acid sequence set forth in SEQ ID NO: 131.

In any of the preceding embodiments, the set of isolated polynucleotides can comprise two amplification primers which comprise nucleic acid sequences set forth in SEQ ID NOs: 75 and 76, respectively, and/or a primer for single base extension which comprises the nucleic acid sequence set forth in SEQ ID NO: 132.

In any of the preceding embodiments, the set of isolated polynucleotides can comprise two amplification primers which comprise nucleic acid sequences set forth in SEQ ID NOs: 77 and 78, respectively, and/or a primer for single base extension which comprises the nucleic acid sequence set forth in SEQ ID NO: 133.

In any of the preceding embodiments, the set of isolated polynucleotides can comprise two amplification primers which comprise nucleic acid sequences set forth in SEQ ID NOs: 79 and 80, respectively, and/or a primer for single base extension which comprises the nucleic acid sequence set forth in SEQ ID NO: 134.

In any of the preceding embodiments, the set of isolated polynucleotides can comprise two amplification primers which comprise nucleic acid sequences set forth in SEQ ID NOs: 81 and 82, respectively, and/or a primer for single base extension which comprises the nucleic acid sequence set forth in SEQ ID NO: 135.

In any of the preceding embodiments, the set of isolated polynucleotides can comprise two amplification primers which comprise nucleic acid sequences set forth in SEQ ID NOs: 83 and 84, respectively, and/or a primer for single base extension which comprises the nucleic acid sequence set forth in SEQ ID NO: 136.

In any of the preceding embodiments, the set of isolated polynucleotides can comprise two amplification primers which comprise nucleic acid sequences set forth in SEQ ID NOs: 85 and 86, respectively, and/or a primer for single base extension which comprises the nucleic acid sequence set forth in SEQ ID NO: 137.

In any of the preceding embodiments, the set of isolated polynucleotides can comprise two amplification primers which comprise nucleic acid sequences set forth in SEQ ID NOs: 87 and 88, respectively, and/or a primer for single base extension which comprises the nucleic acid sequence set forth in SEQ ID NO: 138.

In any of the preceding embodiments, the set of isolated polynucleotides can comprise two amplification primers which comprise nucleic acid sequences set forth in SEQ ID NOs: 89 and 90, respectively, and/or a primer for single base extension which comprises the nucleic acid sequence set forth in SEQ ID NO: 139.

In any of the preceding embodiments, the set of isolated polynucleotides can comprise two amplification primers which comprise nucleic acid sequences set forth in SEQ ID NOs: 91 and 92, respectively, and/or a primer for single base extension which comprises the nucleic acid sequence set forth in SEQ ID NO: 140.

In any of the preceding embodiments, the set of isolated polynucleotides can comprise two amplification primers which comprise nucleic acid sequences set forth in SEQ ID NOs: 93 and 94, respectively, and/or a primer for single base extension which comprises the nucleic acid sequence set forth in SEQ ID NO: 141.

In any of the preceding embodiments, the isolated polynucleotide or set of isolated polynucleotides can be for detection of one or more SNPs associated with diabetes mellitus and/or a disease or condition related to diabetes mellitus, for example, for detection of one or more SNPs associated with type 2 diabetes mellitus, diabetic nephropathy, diabetic retinitis, diabetic cardiomyopathy (e.g., elderly diabetic cardiomyopathy), and/or drug resistance to an anti-diabetes medication. In one aspect, the SNPs associated with type 2 diabetes mellitus comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329. In another aspect, the SNPs associated with diabetic nephropathy comprise any one or more of rs2268388 and rs1801282. In one aspect, the SNPs associated with diabetic retinitis comprise any one or more of rs9565164, rs4668142, rs2380261, rs39059, rs17756941, rs245955, and rs245962. In another aspect, the SNPs associated with diabetic cardiomyopathy comprise any one or more of rs266729, rs3811951, rs156019, rs6234, rs1801282, and rs3856806. In still another aspect, the SNPs associated with the drug resistance comprise any one or more of rs13266634, rs10494366, rs2237892, rs11977021, rs7651090, rs5219, rs7903146, rs1801282, rs6467136, rs2230806, rs11212617, and rs622342.

In any of the preceding embodiments, the anti-diabetes medication can comprise any one or more of Repaglinide, Rosiglitazone, Metformin, Gliclazide, and Pioglitazone. In any of the preceding embodiments, the SNPs can be associated with Repaglinide resistance. In one aspect, the SNPs can comprise any one or more of rs13266634, rs10494366, rs2237892, rs11977021, rs7651090, rs5219, and rs7903146. In any of the preceding embodiments, the SNPs can be associated with Rosiglitazone resistance. In one aspect, the SNPs can comprise any one or more of rs13266634, rs2237892, rs1801282, rs6467136, and rs2230806. In any of the preceding embodiments, the SNPs can be associated with Metformin resistance. In one aspect, the SNPs can comprise any one or more of rs11212617 and rs622342. In any of the preceding embodiments, the SNPs can be associated with Gliclazide resistance. In one aspect, the SNPs can comprise rs5219. In any of the preceding embodiments, the SNPs can be associated with Pioglitazone resistance. In one aspect, the SNPs can comprise rs1801282.

In any of the preceding embodiments, the diabetes mellitus and/or disease or condition related to diabetes mellitus can be in an East Asian population.

In another aspect, disclosed herein is a kit comprising the isolated polynucleotide or the set of isolated polynucleotides of any of the preceding embodiments. In one embodiment, the kit further comprises instructions for using the isolated polynucleotide or the set of isolated polynucleotides to conduct a companion diagnostic test. In one embodiment, the companion diagnostic test is for treatment of diabetes mellitus and/or a disease or condition related to diabetes mellitus.

In yet another aspect, disclosed herein is a use of the isolated polynucleotide, the set of isolated polynucleotides, or the kit of any of the preceding embodiments, for detecting one or more of the SNPs selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941 rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

In still another aspect, disclosed herein is a use of the isolated polynucleotide, the set of isolated polynucleotides, or the kit of any of the preceding embodiments, for the manufacture of a product for detecting one or more of the SNPs selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

In another aspect, discloses herein is a method for risk assessment, diagnosis, prognosis and/or treatment monitoring of diabetes mellitus and/or a disease or condition related to diabetes mellitus in a subject, the method comprising detecting one or more single nucleotide polymorphisms (SNPs) in a biological sample from the subject, wherein the SNPs are selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

In one embodiment, the one or more SNPs are detected using a primer for the SNP or SNPs. In one aspect, the primer comprises the nucleic acid sequence set forth in any of SEQ ID NOs: 1-141. In another aspect, a plurality of primers are used to detect the SNP or SNPs, and the plurality of primers comprise at least two, or at least three, of the nucleic acid sequences set forth in SEQ ID NOs: 1-141. In one embodiment, the primer comprises one or more, or two or more, of the nucleic acid sequences set forth in SEQ ID NOs: 1-94. In another embodiment, the primer comprises one or more of the nucleic acid sequences set forth in SEQ ID NOs: 95-141.

In any of the preceding embodiments, the SNP or SNPs can be detected using at least two amplification primers. In any of the preceding embodiments, the SNP or SNPs can be detected using at least one primer for single base extension.

In any of the preceding embodiments, the SNP or SNPs can be detected using:

two amplification primers comprising nucleic acid sequences set forth in SEQ ID NOs: 1 and 2, respectively, and/or a primer for single base extension comprising the nucleic acid sequence set forth in SEQ ID NO: 95; and/or

two amplification primers comprising nucleic acid sequences set forth in SEQ ID NOs: 3 and 4, respectively, and/or a primer for single base extension comprising the nucleic acid sequence set forth in SEQ ID NO: 96; and/or

two amplification primers comprising nucleic acid sequences set forth in SEQ ID NOs: 5 and 6, respectively, and/or a primer for single base extension comprising the nucleic acid sequence set forth in SEQ ID NO: 97; and/or

two amplification primers comprising nucleic acid sequences set forth in SEQ ID NOs: 7 and 8, respectively, and/or a primer for single base extension comprising the nucleic acid sequence set forth in SEQ ID NO: 98; and/or

two amplification primers comprising nucleic acid sequences set forth in SEQ ID NOs: 9 and 10, respectively, and/or a primer for single base extension comprising the nucleic acid sequence set forth in SEQ ID NO: 99; and/or

two amplification primers comprising nucleic acid sequences set forth in SEQ ID NOs: 11 and 12, respectively, and/or a primer for single base extension comprising the nucleic acid sequence set forth in SEQ ID NO: 100; and/or

two amplification primers comprising nucleic acid sequences set forth in SEQ ID NOs: 13 and 14, respectively, and/or a primer for single base extension comprising the nucleic acid sequence set forth in SEQ ID NO: 101; and/or

two amplification primers comprising nucleic acid sequences set forth in SEQ ID NOs: 15 and 16, respectively, and/or a primer for single base extension comprising the nucleic acid sequence set forth in SEQ ID NO: 102; and/or

two amplification primers comprising nucleic acid sequences set forth in SEQ ID NOs: 17 and 18, respectively, and/or a primer for single base extension comprising the nucleic acid sequence set forth in SEQ ID NO: 103; and/or

two amplification primers comprising nucleic acid sequences set forth in SEQ ID NOs: 19 and 20, respectively, and/or a primer for single base extension comprising the nucleic acid sequence set forth in SEQ ID NO: 104; and/or

two amplification primers comprising nucleic acid sequences set forth in SEQ ID NOs: 21 and 22, respectively, and/or a primer for single base extension comprising the nucleic acid sequence set forth in SEQ ID NO: 105; and/or

two amplification primers comprising nucleic acid sequences set forth in SEQ ID NOs: 23 and 24, respectively, and/or a primer for single base extension comprising the nucleic acid sequence set forth in SEQ ID NO: 106; and/or

two amplification primers comprising nucleic acid sequences set forth in SEQ ID NOs: 25 and 26, respectively, and/or a primer for single base extension comprising the nucleic acid sequence set forth in SEQ ID NO: 107; and/or

two amplification primers comprising nucleic acid sequences set forth in SEQ ID NOs: 27 and 28, respectively, and/or a primer for single base extension comprising the nucleic acid sequence set forth in SEQ ID NO: 108; and/or

two amplification primers comprising nucleic acid sequences set forth in SEQ ID NOs: 29 and 30, respectively, and/or a primer for single base extension comprising the nucleic acid sequence set forth in SEQ ID NO: 109; and/or

two amplification primers comprising nucleic acid sequences set forth in SEQ ID NOs: 31 and 32, respectively, and/or a primer for single base extension comprising the nucleic acid sequence set forth in SEQ ID NO: 110; and/or

two amplification primers comprising nucleic acid sequences set forth in SEQ ID NOs: 33 and 34, respectively, and/or a primer for single base extension comprising the nucleic acid sequence set forth in SEQ ID NO: 111; and/or

two amplification primers comprising nucleic acid sequences set forth in SEQ ID NOs: 35 and 36, respectively, and/or a primer for single base extension comprising the nucleic acid sequence set forth in SEQ ID NO: 112; and/or

two amplification primers comprising nucleic acid sequences set forth in SEQ ID NOs: 37 and 38, respectively, and/or a primer for single base extension comprising the nucleic acid sequence set forth in SEQ ID NO: 113; and/or

two amplification primers comprising nucleic acid sequences set forth in SEQ ID NOs: 39 and 40, respectively, and/or a primer for single base extension comprising the nucleic acid sequence set forth in SEQ ID NO: 114; and/or

two amplification primers comprising nucleic acid sequences set forth in SEQ ID NOs: 41 and 42, respectively, and/or a primer for single base extension comprising the nucleic acid sequence set forth in SEQ ID NO: 115; and/or

two amplification primers comprising nucleic acid sequences set forth in SEQ ID NOs: 43 and 44, respectively, and/or a primer for single base extension comprising the nucleic acid sequence set forth in SEQ ID NO: 116; and/or

two amplification primers comprising nucleic acid sequences set forth in SEQ ID NOs: 45 and 46, respectively, and/or a primer for single base extension comprising the nucleic acid sequence set forth in SEQ ID NO: 117; and/or

two amplification primers comprising nucleic acid sequences set forth in SEQ ID NOs: 47 and 48, respectively, and/or a primer for single base extension comprising the nucleic acid sequence set forth in SEQ ID NO: 118; and/or

two amplification primers comprising nucleic acid sequences set forth in SEQ ID NOs: 49 and 50, respectively, and/or a primer for single base extension comprising the nucleic acid sequence set forth in SEQ ID NO: 119; and/or

two amplification primers comprising nucleic acid sequences set forth in SEQ ID NOs: 51 and 52, respectively, and/or a primer for single base extension comprising the nucleic acid sequence set forth in SEQ ID NO: 120; and/or

two amplification primers comprising nucleic acid sequences set forth in SEQ ID NOs: 53 and 54, respectively, and/or a primer for single base extension comprising the nucleic acid sequence set forth in SEQ ID NO: 121; and/or

two amplification primers comprising nucleic acid sequences set forth in SEQ ID NOs: 55 and 56, respectively, and/or a primer for single base extension comprising the nucleic acid sequence set forth in SEQ ID NO: 122; and/or

two amplification primers comprising nucleic acid sequences set forth in SEQ ID NOs: 57 and 58, respectively, and/or a primer for single base extension comprising the nucleic acid sequence set forth in SEQ ID NO: 123; and/or

two amplification primers comprising nucleic acid sequences set forth in SEQ ID NOs: 59 and 60, respectively, and/or a primer for single base extension comprising the nucleic acid sequence set forth in SEQ ID NO: 124; and/or

two amplification primers comprising nucleic acid sequences set forth in SEQ ID NOs: 61 and 62, respectively, and/or a primer for single base extension comprising the nucleic acid sequence set forth in SEQ ID NO: 125; and/or

two amplification primers comprising nucleic acid sequences set forth in SEQ ID NOs: 63 and 64, respectively, and/or a primer for single base extension comprising the nucleic acid sequence set forth in SEQ ID NO: 126; and/or

two amplification primers comprising nucleic acid sequences set forth in SEQ ID NOs: 65 and 66, respectively, and/or a primer for single base extension comprising the nucleic acid sequence set forth in SEQ ID NO: 127; and/or

two amplification primers comprising nucleic acid sequences set forth in SEQ ID NOs: 67 and 68, respectively, and/or a primer for single base extension comprising the nucleic acid sequence set forth in SEQ ID NO: 128; and/or

two amplification primers comprising nucleic acid sequences set forth in SEQ ID NOs: 69 and 70, respectively, and/or a primer for single base extension comprising the nucleic acid sequence set forth in SEQ ID NO: 129; and/or

two amplification primers comprising nucleic acid sequences set forth in SEQ ID NOs: 71 and 72, respectively, and/or a primer for single base extension comprising the nucleic acid sequence set forth in SEQ ID NO: 130; and/or

two amplification primers comprising nucleic acid sequences set forth in SEQ ID NOs: 73 and 74, respectively, and/or a primer for single base extension comprising the nucleic acid sequence set forth in SEQ ID NO: 131; and/or

two amplification primers comprising nucleic acid sequences set forth in SEQ ID NOs: 75 and 76, respectively, and/or a primer for single base extension comprising the nucleic acid sequence set forth in SEQ ID NO: 132; and/or

two amplification primers comprising nucleic acid sequences set forth in SEQ ID NOs: 77 and 78, respectively, and/or a primer for single base extension comprising the nucleic acid sequence set forth in SEQ ID NO: 133; and/or

two amplification primers comprising nucleic acid sequences set forth in SEQ ID NOs: 79 and 80, respectively, and/or a primer for single base extension comprising the nucleic acid sequence set forth in SEQ ID NO: 134; and/or

two amplification primers comprising nucleic acid sequences set forth in SEQ ID NOs: 81 and 82, respectively, and/or a primer for single base extension comprising the nucleic acid sequence set forth in SEQ ID NO: 135; and/or

two amplification primers comprising nucleic acid sequences set forth in SEQ ID NOs: 83 and 84, respectively, and/or a primer for single base extension comprising the nucleic acid sequence set forth in SEQ ID NO: 136; and/or

two amplification primers comprising nucleic acid sequences set forth in SEQ ID NOs: 85 and 86, respectively, and/or a primer for single base extension comprising the nucleic acid sequence set forth in SEQ ID NO: 137; and/or

two amplification primers comprising nucleic acid sequences set forth in SEQ ID NOs: 87 and 88, respectively, and/or a primer for single base extension comprising the nucleic acid sequence set forth in SEQ ID NO: 138; and/or

two amplification primers comprising nucleic acid sequences set forth in SEQ ID NOs: 89 and 90, respectively, and/or a primer for single base extension comprising the nucleic acid sequence set forth in SEQ ID NO: 139; and/or

two amplification primers comprising nucleic acid sequences set forth in SEQ ID NOs: 91 and 92, respectively, and/or a primer for single base extension comprising the nucleic acid sequence set forth in SEQ ID NO: 140; and/or

two amplification primers comprising nucleic acid sequences set forth in SEQ ID NOs: 93 and 94, respectively, and/or a primer for single base extension comprising the nucleic acid sequence set forth in SEQ ID NO: 141.

In any of the preceding embodiments, the one or more SNPs can be associated with type 2 diabetes mellitus, diabetic nephropathy, diabetic retinitis, diabetic cardiomyopathy (e.g., elderly diabetic cardiomyopathy), and/or drug resistance to an anti-diabetes medication. In any of the preceding embodiments, the diabetes mellitus and/or disease or condition related to diabetes mellitus can comprise type 2 diabetes mellitus, diabetic nephropathy, diabetic retinitis, diabetic cardiomyopathy (e.g., elderly diabetic cardiomyopathy), and/or drug resistance to an anti-diabetes medication.

In any of the preceding embodiments, the SNPs associated with type 2 diabetes mellitus can comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329. In any of the preceding embodiments, the SNPs associated with diabetic nephropathy can comprise any one or more of rs2268388 and rs1801282. In any of the preceding embodiments, the SNPs associated with diabetic retinitis can comprise any one or more of rs9565164, rs4668142, rs2380261, rs39059, rs17756941, rs245955, and rs245962. In any of the preceding embodiments, the SNPs associated with diabetic cardiomyopathy can comprise any one or more of rs266729, rs3811951, rs156019, rs6234, rs1801282, and rs3856806. In any of the preceding embodiments, the SNPs associated with the drug resistance can comprise any one or more of rs13266634, rs10494366, rs2237892, rs11977021, rs7651090, rs5219, rs7903146, rs1801282, rs6467136, rs2230806, rs11212617, and rs622342. In any of the preceding embodiments, the anti-diabetes medication can comprise any one or more of Repaglinide, Rosiglitazone, Metformin. Gliclazide, and Pioglitazone. In any of the preceding embodiments, the SNPs associated with Repaglinide resistance can comprise any one or more of rs13266634, rs10494366, rs2237892, rs11977021, rs7651090, rs5219, and rs7903146. In any of the preceding embodiments, the SNPs associated with Rosiglitazone resistance can comprise any one or more of rs13266634, rs2237892, rs1801282, rs6467136, and rs2230806. In any of the preceding embodiments, the SNPs associated with Metformin resistance can comprise any one or more of rs11212617 and rs622342. In any of the preceding embodiments, the SNPs associated with Gliclazide resistance can comprise rs5219. In any of the preceding embodiments, the SNPs associated with Pioglitazone resistance can comprise rs1801282.

In any of the preceding embodiments, the SNPs can be associated with diabetes mellitus and/or a disease or condition related to diabetes mellitus in an East Asian population.

In some embodiments, the present method can further comprise treating diabetes mellitus and/or a disease or condition related to diabetes mellitus in the subject. In other embodiments, the treatment of diabetes mellitus and/or a disease or condition related to diabetes mellitus in the subject is adjusted based on the SNP(s) detection result in the biological sample from the subject.

The present methods can be used to detect SNP(s) in a biological sample from any suitable subject. For example, the subject can be a human. In another example, the subject can be a non-human mammal. In still another example, the subject can be a non-human animal. Exemplary non-human animals include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications referred to in this application are incorporated by reference in their entireties for all purposes to the same extent as if each individual publication were individually incorporated by reference.

All headings are for the convenience of the reader and should not be used to limit the meaning of the text that follows the heading, unless so specified.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Anazvsis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryolic Genes (1990), Bartlett Publ.: M. Kriegler, Gene Transfer and Expression (1990). Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

### DEFINITIONS

As used herein, the singular forms “a”, “an”, and “the” include plural references unless indicated otherwise. For example, “a” sample includes one or more samples, and “a” primer includes one or more primers.

It is understood that aspects and embodiments of the disclosure described herein include “consisting” and/or “consisting essentially of” aspects and embodiments.

The term “biomarker” or “marker” as used herein refers generally to a molecule, including a gene, protein, carbohydrate structure, or glycolipid, the expression of which in or on a mammalian tissue or cell or secreted can be detected by known methods (or methods disclosed herein) and is predictive or can be used to predict (or aid prediction) for a mammalian cell's or tissue's sensitivity to, and in some embodiments, to predict (or aid prediction) an individual's responsiveness to treatment regimens. A marker or biomarker herein can be a pharmacogenomic biomarker.

As used herein, a “pharmacogenomic biomarker” is an objective biomarker which correlates with a specific clinical drug response or susceptibility in a subject (see. e.g., McLeod et al., Eur. J. Cancer (1999) 35:1650-1652). It may be a biochemical biomarker, or a clinical sign or symptom. The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of DNA, RNA, or protein for specific tumor markers in a subject, a drug or course of treatment may be selected that is optimized for the treatment of the specific tumor likely to be present in the subject. Similarly, the presence or absence of a specific sequence mutation or polymorphism may correlate with drug response. The use of pharmacogenomic biomarkers therefore permits the application of the most appropriate treatment for each subject without having to administer the therapy.

As used herein, the term “polymorphic locus” refers to a region in a nucleic acid at which two or more alternative nucleotide sequences are observed in a significant number of nucleic acid samples from a population of individuals. A polymorphic locus may be a nucleotide sequence of two or more nucleotides, an inserted nucleotide or nucleotide sequence, a deleted nucleotide or nucleotide sequence, or a microsatellite, for example. A polymorphic locus that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic locus is often one nucleotide in length, which is referred to herein as a “single nucleotide polymorphism” or a “SNP.”

Where there are two, three, or four alternative nucleotide sequences at a polymorphic locus, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In genetic analysis that identifies one or more pharmacogenomic biomarkers, samples from individuals having different values in a relevant phenotype often are allelotyped and/or genotyped. The term “allelotype” as used herein refers to a process for determining the allele frequency for a polymorphic variant in pooled DNA samples from cases and controls. By pooling DNA from each group, an allele frequency for each locus in each group is calculated. These allele frequencies are then compared to one another.

A genotype or polymorphic variant may be expressed in terms of a “haplotype,” which as used herein refers to a set of DNA variations, or polymorphisms, that tend to be inherited together. A haplotype can refer to a combination of alleles or to a set of SNPs found on the same chromosome. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “clinical sample” or “disease sample” and variations thereof refer to any sample obtained from a subject of interest that would be expected or is known to contain the cellular and/or molecular entity that is to be characterized.

In certain aspects of the present disclosure, a biological sample or material can be obtained and used, and can refer to any sample or material obtained from a living or viral (or prion) source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. A template for a PCR reaction using one or more primers disclosed herein can be comprised in and/or obtained from a biological sample, for example, a sample from a patient or a subject suspected of respiratory tract infection. Biological samples include, but are not limited to, body fluids, such as blood, plasma, serum, cerebrospinal fluid, synovial fluid, urine and sweat, tissue and organ samples from animals and plants and processed samples derived therefrom.

The terms “polynucleotide,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acid (“PNA”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

As disclosed herein, two nucleic acid sequences can have at least 500%/o sequence identity or homology. Preferably, the two nucleic acid sequences have at least 60%, 70%, 80%, 90%, 95%, 96%, 97%, 98%, 99% or 100% of sequence identity or homology. “Complementary or matched” means that two nucleic acid sequences can hybridize under low, middle and/or high stringency condition(s). The percentage of sequence identity or homology is calculated by comparing one to another when aligned to corresponding portions of the reference sequence.

As used herein, “substantially identical” means that two nucleic acid sequences have at least 90% sequence identity or homology. Preferably, the two nucleic acid sequences have at least 95%, 96%, 97%, 98%, 99% or 100% of sequence identity. The percentage of sequence identity or homology is calculated by comparing one to another when aligned to corresponding portions of the reference sequence.

The terms “complementary” and “substantially complementary” include the hybridization or base pairing or the formation of a duplex between nucleotides or nucleic acids, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%. In one aspect, two complementary sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized.

In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, EDTA) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989), and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

The terms “homologous”, “substantially homologous”, and “substantial homology” as used herein denote a sequence of amino acids having at least 50%, 60%, 70%, 80% or 90% identity wherein one sequence is compared to a reference sequence of amino acids. The percentage of sequence identity or homology is calculated by comparing one to another when aligned to corresponding portions of the reference sequence.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a polymerase, for example, a DNA polymerase.

“Amplification,” as used herein, generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

“Responsiveness” can be assessed using any endpoint indicating a benefit to the patient, including, without limitation, (1) inhibition, to some extent, of disease progression, including slowing down and complete arrest; (2) reduction in the number of disease episodes and/or symptoms, (3) reduction in lesional size; (4) inhibition (i.e., reduction, slowing down or complete stopping) of disease cell infiltration into adjacent peripheral organs and/or tissues; (5) inhibition (i.e., reduction, slowing down or complete stopping) of disease spread; (6) relief, to some extent, of one or more symptoms associated with the disorder; (7) increase in the length of disease-free presentation following treatment; (8) decreased mortality at a given point of time following treatment, and/or (9) lack of adverse effects following treatment. Responsiveness can also be assessed using any endpoint indicating side effect and/or toxicity to the patient.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. For example, significant reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

The term “prediction” or “prognosis” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

### Primers for Amplification of SNPs

In one aspect, the present disclosure provides sets of primers to detect one or more of the following SNPs: rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342.

In one aspect, the present disclosure provides primer sets 1-47 for detecting one or more of the following SNPs: rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342.

In one aspect, Primer Set 1 comprises Amplification Primer Set 1 for detecting rs10229583; Primer Set 2 comprises Amplification Primer Set 2 for detecting rs10811661; Primer Set 3 comprises Amplification Primer Set 3 for detecting rs10886471; Primer Set 4 comprises Amplification Primer Set 4 for detecting rs1111875; Primer Set 5 comprises Amplification Primer Set 5 for detecting rs12742393; Primer Set 6 comprises Amplification Primer Set 6 for detecting rs12779790; Primer Set 7 comprises Amplification Primer Set 7 for detecting rs13266634; Primer Set 8 comprises Amplification Primer Set 8 for detecting rs1535500; Primer Set 9 comprises Amplification Primer Set 9 for detecting rs16856187; Primer Set 10 comprises Amplification Primer Set 10 for detecting rs16861329; Primer Set 11 comprises Amplification Primer Set 11 for detecting rs1801282; Primer Set 12 comprises Amplification Primer Set 12 for detecting rs2237892; Primer Set 13 comprises Amplification Primer Set 13 for detecting rs340874; Primer Set 14 comprises Amplification Primer Set 14 for detecting rs3786897; Primer Set 15 comprises Amplification Primer Set 15 for detecting rs4430796; Primer Set 16 comprises Amplification Primer Set 16 for detecting rs5219; Primer Set 17 comprises Amplification Primer Set 17 for detecting rs6017317; Primer Set 18 comprises Amplification Primer Set 18 for detecting rs6467136; Primer Set 19 comprises Amplification Primer Set 19 for detecting rs6815464; Primer Set 20 comprises Amplification Primer Set 20 for detecting rs7041847; Primer Set 21 comprises Amplification Primer Set 21 for detecting rs7172432; Primer Set 22 comprises Amplification Primer Set 22 for detecting rs7612463; Primer Set 23 comprises Amplification Primer Set 23 for detecting rs7651090; Primer Set 24 comprises Amplification Primer Set 24 for detecting rs7756992; Primer Set 25 comprises Amplification Primer Set 25 for detecting rs780094; Primer Set 26 comprises Amplification Primer Set 26 for detecting rs7903146; Primer Set 27 comprises Amplification Primer Set 27 for detecting rs8050136; Primer Set 28 comprises Amplification Primer Set 28 for detecting rs831571; Primer Set 29 comprises Amplification Primer Set 29 for detecting rs9470794; Primer Set 30 comprises Amplification Primer Set 30 for detecting rs2268388; Primer Set 31 comprises Amplification Primer Set 31 for detecting rs9565164; Primer Set 32 comprises Amplification Primer Set 32 for detecting rs4668142; Primer Set 33 comprises Amplification Primer Set 33 for detecting rs2380261; Primer Set 34 comprises Amplification Primer Set 34 for detecting rs39059; Primer Set 35 comprises Amplification Primer Set 35 for detecting rs17756941; Primer Set 36 comprises Amplification Primer Set 36 for detecting rs245955; Primer Set 37 comprises Amplification Primer Set 37 for detecting rs245962; Primer Set 38 comprises Amplification Primer Set 38 for detecting rs266729; Primer Set 39 comprises Amplification Primer Set 39 for detecting rs3811951; Primer Set 40 comprises Amplification Primer Set 40 for detecting rs156019; Primer Set 41 comprises Amplification Primer Set 41 for detecting rs6234; Primer Set 42 comprises Amplification Primer Set 42 for detecting rs3856806; Primer Set 43 comprises Amplification Primer Set 43 for detecting rs10494366; Primer Set 44 comprises Amplification Primer Set 44 for detecting rs11977021; Primer Set 45 comprises Amplification Primer Set 45 for detecting rs2230806; Primer Set 46 comprises Amplification Primer Set 46 for detecting rs11212617; and/or Primer Set 47 comprises Amplification Primer Set 47 for detecting rs622342.

In one aspect, Amplification Primer Set 1 for detecting rs10229583 comprises a single-stranded DNA molecule (e.g., SEQ ID NO: 1) and/or a single-stranded DNA molecule (e.g., SEQ ID NO: 2). In one aspect, Amplification Primer Set 2 for detecting rs10811661 comprises a single-stranded DNA molecule (e.g., SEQ ID NO: 3) and/or a single-stranded DNA molecule (e.g., SEQ ID NO: 4). In one aspect, Amplification Primer Set 3 for detecting rs10886471 comprises a single-stranded DNA molecule (e.g., SEQ ID NO: 5) and/or a single-stranded DNA molecule (e.g., SEQ ID NO: 6). In one aspect, Amplification Primer Set 4 for detecting rs1111875 comprises a single-stranded DNA molecule (e.g., SEQ ID NO: 7) and/or a single-stranded DNA molecule (e.g., SEQ ID NO: 8). In one aspect, Amplification Primer Set 5 for detecting rs12742393 comprises a single-stranded DNA molecule (e.g., SEQ ID NO: 9) and/or a single-stranded DNA molecule (e.g., SEQ ID NO: 10). In one aspect, Amplification Primer Set 6 for detecting rs12779790 comprises a single-stranded DNA molecule (e.g., SEQ ID NO: 11) and/or a single-stranded DNA molecule (e.g., SEQ ID NO: 12). In one aspect, Amplification Primer Set 7 for detecting rs13266634 comprises a single-stranded DNA molecule (e.g., SEQ ID NO: 13) and/or a single-stranded DNA molecule (e.g., SEQ ID NO: 14). In one aspect, Amplification Primer Set 8 for detecting rs1535500 comprises a single-stranded DNA molecule (e.g., SEQ ID NO: 15) and/or a single-stranded DNA molecule (e.g., SEQ ID NO: 16). In one aspect, Amplification Primer Set 9 for detecting rs16856187 comprises a single-stranded DNA molecule (e.g., SEQ ID NO: 17) and/or a single-stranded DNA molecule (e.g., SEQ ID NO: 18). In one aspect, Amplification Primer Set 10 for detecting rs16861329 comprises a single-stranded DNA molecule (e.g., SEQ ID NO: 19) and/or a single-stranded DNA molecule (e.g., SEQ ID NO: 20). In one aspect, Amplification Primer Set 11 for detecting rs1801282 comprises a single-stranded DNA molecule (e.g., SEQ ID NO: 21) and/or a single-stranded DNA molecule (e.g., SEQ ID NO: 22). In one aspect, Amplification Primer Set 12 for detecting rs2237892 comprises a single-stranded DNA molecule (e.g., SEQ ID NO: 23) and/or a single-stranded DNA molecule (e.g., SEQ ID NO: 24). In one aspect, Amplification Primer Set 13 for detecting rs340874 comprises a single-stranded DNA molecule (e.g., SEQ ID NO: 25) and/or a single-stranded DNA molecule (e.g., SEQ ID NO: 26). In one aspect, Amplification Primer Set 14 for detecting rs3786897 comprises a single-stranded DNA molecule (e.g., SEQ ID NO: 27) and/or a single-stranded DNA molecule (e.g., SEQ ID NO: 28). In one aspect, Amplification Primer Set 15 for detecting rs4430796 comprises a single-stranded DNA molecule (e.g., SEQ ID NO: 29) and/or a single-stranded DNA molecule (e.g., SEQ ID NO: 30). In one aspect, Amplification Primer Set 16 for detecting rs5219 comprises a single-stranded DNA molecule (e.g., SEQ ID NO: 31) and/or a single-stranded DNA molecule (e.g., SEQ ID NO: 32). In one aspect, Amplification Primer Set 17 for detecting rs6017317 comprises a single-stranded DNA molecule (e.g., SEQ ID NO: 33) and/or a single-stranded DNA molecule (e.g., SEQ ID NO: 34). In one aspect, Amplification Primer Set 18 for detecting rs6467136 comprises a single-stranded DNA molecule (e.g., SEQ ID NO: 35) and/or a single-stranded DNA molecule (e.g., SEQ ID NO: 36). In one aspect, Amplification Primer Set 19 for detecting rs6815464 comprises a single-stranded DNA molecule (e.g., SEQ ID NO: 37) and/or a single-stranded DNA molecule (e.g., SEQ ID NO: 38). In one aspect, Amplification Primer Set 20 for detecting rs7041847 comprises a single-stranded DNA molecule (e.g., SEQ ID NO: 39) and/or a single-stranded DNA molecule (e.g., SEQ ID NO: 40). In one aspect, Amplification Primer Set 21 for detecting rs7172432 comprises a single-stranded DNA molecule (e.g., SEQ ID NO: 41) and/or a single-stranded DNA molecule (e.g., SEQ ID NO: 42). In one aspect, Amplification Primer Set 22 for detecting rs7612463 comprises a single-stranded DNA molecule (e.g., SEQ ID NO: 43) and/or a single-stranded DNA molecule (e.g., SEQ ID NO: 44). In one aspect, Amplification Primer Set 23 for detecting rs7651090 comprises a single-stranded DNA molecule (e.g., SEQ ID NO: 45) and/or a single-stranded DNA molecule (e.g., SEQ ID NO: 46). In one aspect, Amplification Primer Set 24 for detecting rs7756992 comprises a single-stranded DNA molecule (e.g., SEQ ID NO: 47) and/or a single-stranded DNA molecule (e.g., SEQ ID NO: 48). In one aspect, Amplification Primer Set 25 for detecting rs780094 comprises a single-stranded DNA molecule (e.g., SEQ ID NO: 49) and/or a single-stranded DNA molecule (e.g., SEQ ID NO: 50). In one aspect, Amplification Primer Set 26 for detecting rs7903146 comprises a single-stranded DNA molecule (e.g., SEQ ID NO: 51) and/or a single-stranded DNA molecule (e.g., SEQ ID NO: 52). In one aspect, Amplification Primer Set 27 for detecting rs8050136 comprises a single-stranded DNA molecule (e.g., SEQ ID NO: 53) and/or a single-stranded DNA molecule (e.g., SEQ ID NO: 54). In one aspect, Amplification Primer Set 28 for detecting rs831571 comprises a single-stranded DNA molecule (e.g., SEQ ID NO: 55) and/or a single-stranded DNA molecule (e.g., SEQ ID NO: 56). In one aspect, Amplification Primer Set 29 for detecting rs9470794 comprises a single-stranded DNA molecule (e.g., SEQ ID NO: 57) and/or a single-stranded DNA molecule (e.g., SEQ ID NO: 58). In one aspect, Amplification Primer Set 30 for detecting rs2268388 comprises a single-stranded DNA molecule (e.g., SEQ ID NO: 59) and/or a single-stranded DNA molecule (e.g., SEQ ID NO: 60). In one aspect. Amplification Primer Set 31 for detecting rs9565164 comprises a single-stranded DNA molecule (e.g., SEQ ID NO: 61) and/or a single-stranded DNA molecule (e.g., SEQ ID NO: 62). In one aspect, Amplification Primer Set 32 for detecting rs4668142 comprises a single-stranded DNA molecule (e.g., SEQ ID NO: 63) and/or a single-stranded DNA molecule (e.g., SEQ ID NO: 64). In one aspect, Amplification Primer Set 33 for detecting rs2380261 comprises a single-stranded DNA molecule (e.g., SEQ ID NO: 65) and/or a single-stranded DNA molecule (e.g., SEQ ID NO: 66). In one aspect, Amplification Primer Set 34 for detecting rs39059 comprises a single-stranded DNA molecule (e.g., SEQ ID NO: 67) and/or a single-stranded DNA molecule (e.g., SEQ ID NO: 68). In one aspect, Amplification Primer Set 35 for detecting rs17756941 comprises a single-stranded DNA molecule (e.g., SEQ ID NO: 69) and/or a single-stranded DNA molecule (e.g., SEQ ID NO: 70). In one aspect, Amplification Primer Set 36 for detecting rs245955 comprises a single-stranded DNA molecule (e.g., SEQ ID NO: 71) and/or a single-stranded DNA molecule (e.g., SEQ ID NO: 72). In one aspect. Amplification Primer Set 37 for detecting rs245962 comprises a single-stranded DNA molecule (e.g., SEQ ID NO: 73) and/or a single-stranded DNA molecule (e.g., SEQ ID NO: 74). In one aspect, Amplification Primer Set 38 for detecting rs266729 comprises a single-stranded DNA molecule (e.g., SEQ ID NO: 75) and/or a single-stranded DNA molecule (e.g., SEQ ID NO: 76). In one aspect, Amplification Primer Set 39 for detecting rs3811951 comprises a single-stranded DNA molecule (e.g., SEQ ID NO: 77) and/or a single-stranded DNA molecule (e.g., SEQ ID NO: 78). In one aspect, Amplification Primer Set 40 for detecting rs156019 comprises a single-stranded DNA molecule (e.g., SEQ ID NO: 79) and/or a single-stranded DNA molecule (e.g., SEQ ID NO: 80). In one aspect, Amplification Primer Set 41 for detecting rs6234 comprises a single-stranded DNA molecule (e.g., SEQ ID NO: 81) and/or a single-stranded DNA molecule (e.g., SEQ ID NO: 82). In one aspect, Amplification Primer Set 42 for detecting rs3856806 comprises a single-stranded DNA molecule (e.g., SEQ ID NO: 83) and/or a single-stranded DNA molecule (e.g., SEQ ID NO: 84). In one aspect, Amplification Primer Set 43 for detecting rs10494366 comprises a single-stranded DNA molecule (e.g., SEQ ID NO: 85) and/or a single-stranded DNA molecule (e.g., SEQ ID NO: 86). In one aspect. Amplification Primer Set 44 for detecting rs11977021 comprises a single-stranded DNA molecule (e.g., SEQ ID NO: 87) and/or a single-stranded DNA molecule (e.g., SEQ ID NO: 88). In one aspect, Amplification Primer Set 45 for detecting rs2230806 comprises a single-stranded DNA molecule (e.g., SEQ ID NO: 89) and/or a single-stranded DNA molecule (e.g., SEQ ID NO: 90). In one aspect, Amplification Primer Set 46 for detecting rs11212617 comprises a single-stranded DNA molecule (e.g., SEQ ID NO: 91) and/or a single-stranded DNA molecule (e.g., SEQ ID NO: 92). In one aspect, Amplification Primer Set 47 for detecting rs622342 comprises a single-stranded DNA molecule (e.g., SEQ ID NO: 93) and/or a single-stranded DNA molecule (e.g., SEQ ID NO: 94).

In any of the preceding embodiments, the amplification primer(s) can comprise a nucleic acid sequence having at least about 50%, at least about 55%, at least about 60%, at least about 65%, at least about 70%, at least about 75%, at least about 80%, at least about 85%, at least about 90%, at least about 95%, at least about 99%, or 100% of any one or more of SEQ ID NOs: 1-94.

### Primers for Single Base Extension Reactions

In any of the preceding embodiments, the primer set can further comprise a polynucleotide for detecting one or more of the following SNPs: rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, by single base extension.

Single-base extension (SBE) is a method for determining the identity of a nucleotide base at a specific position along a nucleic acid, for example, for identifying a single-nucleotide polymorphism. In the method, an oligonucleotide primer hybridizes to a complementary region along the nucleic acid, to form a duplex, with the primer's terminal 3′ end directly adjacent to the nucleotide base to be identified. The oligonucleotide primer is enzymatically extended by a single base in the presence of all four nucleotide terminators; the nucleotide terminator complementary to the base in the template being interrogated is incorporated and identified. The presence of all four terminators ensures that no further extension occurs beyond the single incorporated base. Many approaches can be taken for determining the identity of a terminator, including fluorescence labeling, mass labeling for mass spectrometry, measuring enzyme activity using a protein moiety, and isotope labeling. This approach was designed for high-throughput SNP genotyping and was originally called “Genetic Bit Analysis” (GBA). See Goelet et al., 1999, U.S. Pat. No. 5,888,819.

In one aspect, Primer Set 1 comprises Single Base Extension Primer 1 for detecting rs10229583; Primer Set 2 comprises Single Base Extension Primer 2 for detecting rs10811661; Primer Set 3 comprises Single Base Extension Primer 3 for detecting rs10886471; Primer Set 4 comprises Single Base Extension Primer 4 for detecting rs1111875 Primer Set 5 comprises Single Base Extension Primer 5 for detecting rs12742393; Primer Set 6 comprises Single Base Extension Primer 6 for detecting rs12779790; Primer Set 7 comprises Single Base Extension Primer 7 for detecting rs13266634; Primer Set 8 comprises Single Base Extension Primer 8 for detecting rs1535500; Primer Set 9 comprises Single Base Extension Primer 9 for detecting rs16856187; Primer Set 10 comprises Single Base Extension Primer 10 for detecting rs16861329; Primer Set 11 comprises Single Base Extension Primer 11 for detecting rs1801282; Primer Set 12 comprises Single Base Extension Primer 12 for detecting rs2237892; Primer Set 13 comprises Single Base Extension Primer 13 for detecting rs340874; Primer Set 14 comprises Single Base Extension Primer 14 for detecting rs3786897; Primer Set 15 comprises Single Base Extension Primer 15 for detecting rs4430796; Primer Set 16 comprises Single Base Extension Primer 16 for detecting rs5219; Primer Set 17 comprises Single Base Extension Primer 17 for detecting rs6017317; Primer Set 18 comprises Single Base Extension Primer 18 for detecting rs6467136; Primer Set 19 comprises Single Base Extension Primer 19 for detecting rs6815464; Primer Set 20 comprises Single Base Extension Primer 20 for detecting rs7041847; Primer Set 21 comprises Single Base Extension Primer 21 for detecting rs7172432; Primer Set 22 comprises Single Base Extension Primer 22 for detecting rs7612463; Primer Set 23 comprises Single Base Extension Primer 23 for detecting rs7651090; Primer Set 24 comprises Single Base Extension Primer 24 for detecting rs7756992; Primer Set 25 comprises Single Base Extension Primer 25 for detecting rs780094; Primer Set 26 comprises Single Base Extension Primer 26 for detecting rs7903146; Primer Set 27 comprises Single Base Extension Primer 27 for detecting rs8050136; Primer Set 28 comprises Single Base Extension Primer 28 for detecting rs831571; Primer Set 29 comprises Single Base Extension Primer 29 for detecting rs9470794; Primer Set 30 comprises Single Base Extension Primer 30 for detecting rs2268388; Primer Set 31 comprises Single Base Extension Primer 31 for detecting rs9565164; Primer Set 32 comprises Single Base Extension Primer 32 for detecting rs4668142; Primer Set 33 comprises Single Base Extension Primer 33 for detecting rs2380261; Primer Set 34 comprises Single Base Extension Primer 34 for detecting rs39059; Primer Set 35 comprises Single Base Extension Primer 35 for detecting rs17756941; Primer Set 36 comprises Single Base Extension Primer 36 for detecting rs245955; Primer Set 37 comprises Single Base Extension Primer 37 for detecting rs245962; Primer Set 38 comprises Single Base Extension Primer 38 for detecting rs266729; Primer Set 39 comprises Single Base Extension Primer 39 for detecting rs3811951; Primer Set 40 comprises Single Base Extension Primer 40 for detecting rs156019; Primer Set 41 comprises Single Base Extension Primer 41 for detecting rs6234; Primer Set 42 comprises Single Base Extension Primer 42 for detecting rs3856806; Primer Set 43 comprises Single Base Extension Primer 43 for detecting rs10494366; Primer Set 44 comprises Single Base Extension Primer 44 for detecting rs11977021; Primer Set 45 comprises Single Base Extension Primer 45 for detecting rs2230806; Primer Set 46 comprises Single Base Extension Primer 46 for detecting rs11212617; and/or Primer Set 47 comprises Single Base Extension Primer 47 for detecting rs622342.

In one aspect, Single Base Extension Primer 1 for detecting rs10229583 comprises a single-stranded DNA molecule (e.g., SEQ ID NO: 95). In one aspect, Single Base Extension Primer 2 for detecting rs10811661 comprises a single-stranded DNA molecule (e.g., SEQ ID NO: 96). In one aspect, Single Base Extension Primer 3 for detecting rs10886471 comprises a single-stranded DNA molecule (e.g., SEQ ID NO: 97). In one aspect, Single Base Extension Primer 4 for detecting rs1111875 comprises a single-stranded DNA molecule (e.g., SEQ ID NO: 98). In one aspect, Single Base Extension Primer 5 for detecting rs12742393 comprises a single-stranded DNA molecule (e.g., SEQ ID NO: 99). In one aspect, Single Base Extension Primer 6 for detecting rs12779790 comprises a single-stranded DNA molecule (e.g., SEQ ID NO: 100). In one aspect, Single Base Extension Primer 7 for detecting rs13266634 comprises a single-stranded DNA molecule (e.g., SEQ ID NO: 101). In one aspect, Single Base Extension Primer 8 for detecting rs1535500 comprises a single-stranded DNA molecule (e.g., SEQ ID NO: 102). In one aspect, Single Base Extension Primer 9 for detecting rs16856187 comprises a single-stranded DNA molecule (e.g., SEQ ID NO: 103). In one aspect, Single Base Extension Primer 10 for detecting rs16861329 comprises a single-stranded DNA molecule (e.g., SEQ ID NO: 104). In one aspect, Single Base Extension Primer 11 for detecting rs1801282 comprises a single-stranded DNA molecule (e.g., SEQ ID NO: 105). In one aspect, Single Base Extension Primer 12 for detecting rs2237892 comprises a single-stranded DNA molecule (e.g., SEQ ID NO: 106). In one aspect, Single Base Extension Primer 13 for detecting rs340874 comprises a single-stranded DNA molecule (e.g., SEQ ID NO: 107). In one aspect, Single Base Extension Primer 14 for detecting rs3786897 comprises a single-stranded DNA molecule (e.g., SEQ ID NO: 108). In one aspect, Single Base Extension Primer 15 for detecting rs4430796 comprises a single-stranded DNA molecule (e.g., SEQ ID NO: 109). In one aspect. Single Base Extension Primer 16 for detecting rs5219 comprises a single-stranded DNA molecule (e.g., SEQ ID NO: 110). In one aspect, Single Base Extension Primer 17 for detecting rs6017317 comprises a single-stranded DNA molecule (e.g., SEQ ID NO: 111). In one aspect, Single Base Extension Primer 18 for detecting rs6467136 comprises a single-stranded DNA molecule (e.g., SEQ ID NO: 112). In one aspect, Single Base Extension Primer 19 for detecting rs6815464 comprises a single-stranded DNA molecule (e.g., SEQ ID NO: 113). In one aspect, Single Base Extension Primer 20 for detecting rs7041847 comprises a single-stranded DNA molecule (e.g., SEQ ID NO: 114). In one aspect, Single Base Extension Primer 21 for detecting rs7172432 comprises a single-stranded DNA molecule (e.g., SEQ ID NO: 115). In one aspect, Single Base Extension Primer 22 for detecting rs7612463 comprises a single-stranded DNA molecule (e.g., SEQ ID NO: 116). In one aspect, Single Base Extension Primer 23 for detecting rs7651090 comprises a single-stranded DNA molecule (e.g., SEQ ID NO: 117). In one aspect, Single Base Extension Primer 24 for detecting rs7756992 comprises a single-stranded DNA molecule (e.g., SEQ ID NO: 118). In one aspect, Single Base Extension Primer 25 for detecting rs780094 comprises a single-stranded DNA molecule (e.g., SEQ ID NO: 119). In one aspect. Single Base Extension Primer 26 for detecting rs7903146 comprises a single-stranded DNA molecule (e.g., SEQ ID NO: 120). In one aspect, Single Base Extension Primer 27 for detecting rs8050136 comprises a single-stranded DNA molecule (e.g., SEQ ID NO: 121). In one aspect, Single Base Extension Primer 28 for detecting rs831571 comprises a single-stranded DNA molecule (e.g., SEQ ID NO: 122). In one aspect, Single Base Extension Primer 29 for detecting rs9470794 comprises a single-stranded DNA molecule (e.g., SEQ ID NO: 123). In one aspect, Single Base Extension Primer 30 for detecting rs2268388 comprises a single-stranded DNA molecule (e.g., SEQ ID NO: 124). In one aspect, Single Base Extension Primer 31 for detecting rs9565164 comprises a single-stranded DNA molecule (e.g., SEQ ID NO: 125). In one aspect, Single Base Extension Primer 32 for detecting rs4668142 comprises a single-stranded DNA molecule (e.g., SEQ ID NO: 126). In one aspect, Single Base Extension Primer 33 for detecting rs2380261 comprises a single-stranded DNA molecule (e.g., SEQ ID NO: 127). In one aspect, Single Base Extension Primer 34 for detecting rs39059 comprises a single-stranded DNA molecule (e.g., SEQ ID NO: 128). In one aspect, Single Base Extension Primer 35 for detecting rs17756941 comprises a single-stranded DNA molecule (e.g., SEQ ID NO: 129). In one aspect. Single Base Extension Primer 36 for detecting rs245955 comprises a single-stranded DNA molecule (e.g., SEQ ID NO: 130). In one aspect, Single Base Extension Primer 37 for detecting rs245962 comprises a single-stranded DNA molecule (e.g., SEQ ID NO: 131). In one aspect, Single Base Extension Primer 38 for detecting rs266729 comprises a single-stranded DNA molecule (e.g., SEQ ID NO: 132). In one aspect, Single Base Extension Primer 39 for detecting rs381195 comprises a single-stranded DNA molecule (e.g., SEQ ID NO: 133). In one aspect, Single Base Extension Primer 40 for detecting rs156019 comprises a single-stranded DNA molecule (e.g., SEQ ID NO: 134). In one aspect, Single Base Extension Primer 41 for detecting rs6234 comprises a single-stranded DNA molecule (e.g., SEQ ID NO: 135). In one aspect, Single Base Extension Primer 42 for detecting rs3856806 comprises a single-stranded DNA molecule (e.g., SEQ ID NO: 136). In one aspect, Single Base Extension Primer 43 for detecting rs10494366 comprises a single-stranded DNA molecule (e.g., SEQ ID NO: 137). In one aspect, Single Base Extension Primer 44 for detecting rs11977021 comprises a single-stranded DNA molecule (e.g., SEQ ID NO: 138). In one aspect, Single Base Extension Primer 45 for detecting rs2230806 comprises a single-stranded DNA molecule (e.g., SEQ ID NO: 139). In one aspect. Single Base Extension Primer 46 for detecting rs11212617 comprises a single-stranded DNA molecule (e.g., SEQ ID NO: 140). In one aspect, Single Base Extension Primer 47 for detecting rs622342 comprises a single-stranded DNA molecule (e.g., SEQ ID NO: 141).

In any of the preceding embodiments, the single base extension primer(s) can comprise a nucleic acid sequence having at least about 50%, at least about 55%, at least about 60%, at least about 65%, at least about 70%, at least about 75%, at least about 80%, at least about 85%, at least about 90° %, at least about 95%, at least about 99%, or 100% of any one or more of SEQ ID NOs: 95-141.

In any of the preceding embodiments, each SNP locus can correspond to a set of amplification primers and a single-base extension primer.

In some embodiments, the rs10229583, the rs10811661, the rs10886471, the rs1111875, the rs12742393, the rs12779790, the rs13266634, the rs1535500, the rs16856187, the rs16861329, the rs1801282, the rs2237892, the rs340874, the rs3786897, the rs4430796, the rs5219, the rs6017317, the rs6467136, the rs6815464, the rs7041847, the rs7172432, the rs7612463, the rs7651090, the rs7756992, the rs780094, the rs7903146, the rs8050136, the rs831571, the rs9470794, the rs2268388, the rs9565164, the rs4668142, the rs2380261, the rs39059, the rs17756941, the rs245955, the rs245962, the rs266729, the rs3811951, the rs156019, the rs6234, the rs3856806, the rs10494366, the rs11977021, the rs2230806, the rs11212617, and the rs622342 markers are the at least two allelic polymorphism SNPs on human chromosome, and the specific location of each SNP and flanking sequences are shown in Tables 1 to 5.

In some aspects, the present disclosure provides several advantages:

1) Risk of patients carrying any risk allele is higher than the general population. In addition, the risk alleles have the additive effect. Thus, the sets of primers of the present disclosure to detect the SNP combination are more accurate than detection of a single site.

2) SNP sites of the present disclosure associated with type 2 diabetes are verified in East Asian population, which is suitable for the prediction, prevention and individualized treatment for type 2 diabetes of Chinese population.

The present disclosure is the first to reveal the SNP loci combination associated with type 2 diabetes in East Asian, diabetic complications susceptibility and antidiabetic drug resistance, and the primers are designed accordingly. The experimental results show the use of the amplification primers and single base extension primers can accurately detect the SNPs associated with type 2 diabetes in East Asian, the complications of and susceptibility to type 2 diabetes, and antidiabetic drug resistance.

### EXAMPLES

Experimental methods used in the embodiments described below are the conventional methods unless otherwise specified.

The materials and reagents used in following embodiments can be obtained from commercial sources unless otherwise specified.

### Example 1

### The Design of the Amplification Primers for the Combination of SNPs Associated with Type 2 Diabetes Mellitus in the East Asian Population, Diabetic Nephropathy, Diabetic Retinitis (DR), Elderly Diabetic Cardiopathy, and Anti-Diabetic Drug Sensitivity

### I. Screening for SNPs Associated with Type 2 Diabetes Mellitus in an East Asian Population, Diabetic Nephropathy, Diabetic Retinitis (DR), Elderly Diabetic Cardiopathy, and Anti-Diabetic Drug Sensitivity

SNPs associated with type 2 diabetes mellitus in East Asians, diabetic nephropathy, diabetic retinitis (DR), elderly diabetic cardiopathy, and drug resistance to anti-diabetic treatment such as Repaglinide ((S)-(+)-2-ethoxy-4-[2-(3-methyl-1-[2-(piperidin-1-yl)phenyl]butylamino)-2-oxoethyl]benzoic acid), Rosiglitazone ((RS)-5-[4-(2-[methyl(pyridin-2-yl)amino]ethoxy)benzyl]thiazolidine-2,4-dione), Metformin (N,N-Dimethylimidodicarbonimidic diamide), and Gliclazide (N-(hexahydrocyclopenta[c]pyrrol-2(1H)-ylcarbamoyl)-4-methylbenzenesulfonamide), are searched and retrieved, and papers describing the same are downloaded from databases, such as PubMed, mtSNP, the National Knowledge Infrastructure (CKNI database), and the Vip database (Chongqing), and so on. SNP alleles that are significantly associated with increased risks for type 2 diabetes mellitus in East Asians, diabetic nephropathy, diabetic retinitis (DR), and elderly diabetic cardiopathy, are selected. In addition, SNP alleles that are significantly associated with drug resistance to anti-diabetic medications are selected.

The SNPs associated with type 2 diabetes in East Asians selected in this example are as shown in Table 1. The SNPs associated with diabetic nephropathy are shown in Table 2. The SNPs associated with diabetic retinitis are shown in Table 3. The SNPs associated with elderly diabetic cardiopathy are shown in Table 4. The SNPs associated with resistance to anti-diabetic drugs (Repaglinide, Rosiglitazone. Metformin and Gliclazide) are shown in Table 5.

TABLE 1
SNPs associated with type 2 diabetes

| | | | | Risk/ | Flanking sequences | |
|---|---|---|---|---|---|---|
| | | | | safe | [the SNP sequence of | |
| | RS NO. | Gene | Locus | allele | alleles] | SEQ ID NO |
| 1 | rs7756992 | CDKAL1 | Chr6:20679478 | G/A | ATATTCCCCCCTGTATTTTA | SEQ ID NO: |
| | | | | | GTTTT[A/G]GATCTACAGT | 165 |
| | | | | | TATGTAGCAATGAGC | |
| 2 | rs10811661 | / | Chr9:22134095 | T/C | CAGCTCACCTCCAGCTTTAG | SEQ ID NO: |
| | | | | | TTTTC[C/T]CATGACAGTA | 143 |
| | | | | | AGTCTATTACCCTCC | |
| 3 | rs8050136 | FTO | Chr16:53782363 | A/C | CATGCCAGTTGCCCACTGTG | SEQ ID NO: |
| | | | | | GCAAT[A/C]AATATCTGAG | 168 |
| | | | | | CCTGTGGTTTTTGCC | |
| 4 | rs7041847 | GLIS3 | Chr9:4287466 | A/G | AGAAGGCCCCTCCTTCCCTT | SEQ ID NO: |
| | | | | | GACCA[A/G]TAGCTCCCCA | 161 |
| | | | | | AAATGTATGCGATGA | |
| 5 | rs1111875 | / | Chr10:92703125 | G/A | TCCGTACCATCAAGTCATTT | SEQ ID NO: |
| | | | | | CCTCT[A/G]GACGTCTGAA | 145 |
| | | | | | CCTGCACTCAGGGTC | |
| 6 | rs4430796 | HNF1β | Chr17:37738049 | G/A | ATACAGAGAGGCAGCACAGA | SEQ ID NO: |
| | | | | | CTGGA[A/G]ATGCTGCATA | 156 |
| | | | | | AAGCTTAAATTGGGC | |
| 7 | rs7651090 | IGF2BP2 | Chr3:185795604 | G/A | GTGAATTCTTTAAGAAAATG | SEQ ID NO: |
| | | | | | AAGCC[A/G]GAAGCAGTGG | 164 |
| | | | | | GTCAGTCTGTAATCC | |
| 8 | rs2237892 | KCNQ1 | Ch 11:2818521 | C/T | GGAGCTGTCACAGGACTTTG | SEQ ID NO: |
| | | | | | CCACC[C/T]GGGGTGAGGG | 153 |
| | | | | | GCCTAGAAACCCCTC | |
| 9 | rs13266634 | SLC30A8 | Chr8:7172544 | C/T | TGCTTCTTTATCAACAGCAG | SEQ ID NO: |
| | | | | | CCAGC[C/T]GGGACAGCCA | 148 |
| | | | | | AGTGGTTCGGAGAGA | |
| 10 | rs1801282 | PPARG | Chr3:12351626 | C/G | AACTCTGGGAGATTCTCCTA | SEQ ID NO: |
| | | | | | TTGAC[C/G]CAGAAAGCGA | 152 |
| | | | | | TTCCTTCACTGATAC | |
| 11 | rs6017317 | / | Chr20:44318326 | G/T | AAGGCAAAGGATCTGAATAG | SEQ ID NO: |
| | | | | | CTATT[G/T]CTCAAAACAA | 158 |
| | | | | | GACAACATAGAAATG | |
| 12 | rs16856187 | G6PC2 | Chr2:168913876 | A/C | CCTCAGTTTACCCAGTTCTC | SEQ ID NO: |
| | | | | | AACTG[A/C]GGAGTTTAGT | 150 |
| | | | | | ATGGGGTTAAAATGG | |
| 13 | rs6467136 | / | Chr7:127524904 | G/A | CTTGGACATTACTTTAAAAG | SEQ ID NO: |
| | | | | | TGCAA[A/G]TGACAAAAGA | 159 |
| | | | | | AAAATATAGATAAAT | |
| 14 | rs5219 | KCNJ11 | Chr11:17388025 | T/C | CGCTGGCGGGCACGGTACCT | SEQ ID NO: |
| | | | | | GGGCT[C/T]GGCAGGGTCC | 157 |
| | | | | | TCTGCCAGGCGTGTC | |
| 15 | rs1535500 | KCNK16 | Chr6:39316274 | T/G | GGGGAGCCCACTGGGGTCAG | SEQ ID NO: |
| | | | | | AGGCT[G/T]CCCCATCCTT | 149 |
| | | | | | GACGCCGAGGCCCCT | |
| 16 | rs6815464 | MAEA | Chr4:1316113 | C/G | CCCGATACCTGTACCCCGGG | SEQ ID NO: |
| | | | | | TTTTG[C/G]GCTGACACAT | 160 |
| | | | | | GCTCCATTGCTTCCT | |
| 17 | rs12742393 | NOS1AP | Chr1:162254796 | C/A | TGGGAACAGGGCAATGCATT | SEQ ID NO: |
| | | | | | TTACC[A/C]GTACAATCTG | 146 |
| | | | | | TGGTATGGGTGGTGT | |
| 18 | rs10229583 | PAX4 | Chr7:127606849 | A/G | GTCTGATTTTTAAATCTGTT | SEQ ID NO: |
| | | | | | GACAA[A/G]AGAAGGCTGA | 142 |
| | | | | | AACTGGAACATAAGA | |
| 19 | rs3786897 | PEPD | Chr19:33402102 | A/G | ACCTGTGTCTTCCAGGGAAA | SEQ ID NO: |
| | | | | | AAGGG[A/G]CTCAGGGCTC | 155 |
| | | | | | AGCCCAGGCAGGGAA | |
| 20 | rs831571 | PSMD6 | Chr3:64062621 | C/T | CCTCTTGACAACAAGATAGG | SEQ ID NO: |
| | | | | | CTTTA[C/T]TTCGCCTCTA | 169 |
| | | | | | GAATGGCCTCCAGCC | |
| 21 | rs7903146 | TCF7L2 | Chr10:112998590 | T/C | TAGAGAGCTAAGCACTTTTT | SEQ ID NO: |
| | | | | | AGATA[C/T]TATATAATTT | 167 |
| | | | | | AATTGCCGTATGAGG | |
| 22 | rs9470794 | ZFAND3 | Chr6:38139068 | C/T | ATGAAGGATTCCAAGGAGCA | SEQ ID NO: |
| | | | | | GCAGT[C/T]GGTCAGGTGG | 170 |
| | | | | | GAAGAAAGGAGTGTG | |
| 23 | rs780094 | GCKR | Chr2:27518370 | G/A | GTTTTTTAGACCATGACTGA | SEQ ID NO: |
| | | | | | CACAT[A/G]TTTGCTGATC | 166 |
| | | | | | AATACATTTGTTGAG | |
| 24 | rs10886471 | GRK5 | Chr10:119389891 | C/T | AGTGCTTGTGTCCCTGGTCT | SEQ ID NO: |
| | | | | | CTGGG[C/T]TCTTGGCCCA | 144 |
| | | | | | GCTGCTTTTCTGATT | |
| 25 | rs12779790 | / | Chr10:12286011 | G/A | CCCGGACAATGTTGGGAATT | SEQ ID NO: |
| | | | | | TTTTC[A/G]TATTTCTTGG | 147 |
| | | | | | CCATTTATATATCTT | |
| 26 | rs340874 | PROX1 | Chr1:213985913 | C/T | AAATAAACTGGTAGGAGTAA | SEQ ID NO: |
| | | | | | GGGCT[A/G]TATACCTTTC | 154 |
| | | | | | CACACCTTAAAACCA | |
| 27 | rs7612463 | UBE2E | Chr3:23294959 | C/A | ATACAGGGTCTTCATTTATT | SEQ ID NO: |
| | | | | | TTTAG[A/C]TACCTGAAAC | 163 |
| | | | | | TGAGTCTAAAACCAC | |
| 28 | rs7172432 | / | Chr15:62104190 | A/G | TGTCACTGTCTACAAAATTG | SEQ ID NO: |
| | | | | | TTAAT[A/G]TTCCCAAAGA | 162 |
| | | | | | AACTGTCTGGGCCCC | |
| 29 | rs16861329 | ST6GAL1 | Chr3:186948673 | T/C | ACTGGTCTCTCTGCTATACC | SEQ ID NO: |
| | | | | | AACAC[C/T]TTTGGCTCAG | 151 |
| | | | | | CCTCAGCTCCAGCCA | |

TABLE 2
SNPs associated with diabetic nephropathy

| | | | | Risk/ | Flanking sequences | |
|---|---|---|---|---|---|---|
| | | | | safe | [the SNP sequence of | |
| | RS NO. | Gene | Locus | allele | alleles] | SEQ ID NO |
| 1 | rs2268388 | ACACB | Chr12:109205840 | T/C | GCTGTTCTCCCAGCAGAGAA | SEQ ID NO: |
| | | | | | CACTC[C/T]GTTTCCTGCC | 171 |
| | | | | | CACCCTCTACCCCTC | |
| 2 | rs1801282 | PPARG | Chr3:1235162 | C/G | AACTCTGGGAGATTCTCCTA | SEQ ID NO: |
| | | | | | TTGAC[C/G]CAGAAAGCGA | 152 |
| | | | | | TTCCTTCACTGATAC | |

TABLE 3
SNPs associated with diabetic retinitis

| | | | | Risk/ | Flanking sequences | |
|---|---|---|---|---|---|---|
| | | | | safe | [the SNP sequence of | |
| | RS NO. | Gene | Locus | allele | alleles] | SEQ ID NO |
| 1 | rs9565164 | / | Chr13:75465240 | C/T | GCTATCAACTAATGCTGTTT | SEQ ID NO: |
| | | | | | TTCAC[C/T]TAGTTTTGTC | 172 |
| | | | | | CCACTCCTCTGCAAA | |
| 2 | rs4668142 | / | Chr2:1694077675 | T/G | TGTTTCATGCCAGCCAATGT | SEQ ID NO: |
| | | | | | TCTGA[G/T]GTTCCAGCAT | 173 |
| | | | | | GCTTCACAGCAAAGC | |
| 3 | rs2380261 | / | Chr2:234732536 | T/G | AGCCCAGCCAAGCATGGCTC | SEQ ID NO: |
| | | | | | CAAGG[G/T]TGAAGAGAGC | 174 |
| | | | | | TGCAAGGAGGCAAGG | |
| 4 | rs39059 | / | Chr7:29215854 | A/G | CTTTTCTTTTGGCTTTAAAT | SEQ ID NO: |
| | | | | | TATGC[A/G]CTCTTCTTCC | 175 |
| | | | | | TTAAGTGACTGATTC | |
| 5 | rs17756941 | / | Chr7:29210391 | G/A | CTTTTGGAATGTGACCTACA | SEQ ID NO: |
| | | | | | CTATG[A/G]AACCCAGCAC | 176 |
| | | | | | GTACACACACAGGCA | |
| 6 | rs245955 | / | Chr7:29236691 | C/T | AATAGCTAACATAATACAAA | SEQ ID NO: |
| | | | | | GTTAC[C/T]AGCTTGCACT | 177 |
| | | | | | TATGTAAGGTCAGAG | |
| 7 | rs245962 | / | Chr7:29250537 | A/G | AGACATGAGACTTTCATGAA | SEQ ID NO: |
| | | | | | TTATA[A/G]CATGACTCCT | 178 |
| | | | | | TAAATAATACAAAAG | |

TABLE 4
SNPs associated with diabetic cardiomyopathy

| | | | | Risk/ | Flanking sequences | |
|---|---|---|---|---|---|---|
| | | | | safe | [the SNP sequence of | |
| | RS NO. | Gene | Locus | allele | alleles] | SEQ ID NO |
| 1 | rs266729 | ADIPOQ | Chr3:186841285 | G/C | GCACGCTCATGTTTTGTTTT | SEQ ID NO: |
| | | | | | TGAAG[C/G]GCAGGATCTG | 179 |
| | | | | | AGCCGGTTCTTGCAA | |
| 2 | rs3811951 | PCSK1 | Chr5:96426773 | G/A | ATGACAATTCAGTGTGTGGT | SEQ ID NO: |
| | | | | | GGAAT[A/G]TTGTTAATGT | 180 |
| | | | | | GAGAGTACTCATGAA | |
| 3 | rs156019 | PCSK1 | Chr5:96411659 | A/T | AAAGCATTTCCGCGCTTGGA | SEQ ID NO: |
| | | | | | AGAAG[A/T]CTGAGTAGTT | 181 |
| | | | | | TTTTACTAGTGTAAA | |
| 4 | rs6234 | PCSK1 | Chr5:96393270 | G/C | TGAGTTGGAGGAGGGAGCCC | SEQ ID NO: |
| | | | | | CTTCC[C/G]AGGCCATGCT | 182 |
| | | | | | GCGACTCCTGCAAAG | |
| 5 | rs1801282 | PPARG | Chr3:12351626 | C/G | AACTCTGGGAGATTCTCCTA | SEQ ID NO: |
| | | | | | TTGAC[C/G]CAGAAAGCGA | 152 |
| | | | | | TTCCTTCACTGATAC | |
| 6 | rs3856806 | PPARγ | Chr3:12434058 | C/T | ACCTCAGACAGATTGTCACG | SEQ ID NO: |
| | | | | | GAACA[C/T]GTGCAGCTAC | 183 |
| | | | | | TGCAGGTGATCAAGA | |

TABLE 5
SNPs associated with drug resistance

| | | | | | | Flanking sequences | |
|---|---|---|---|---|---|---|---|
| | | | | Good drug | Poor drug | [the SNP sequence of | |
| Drug | SNP | Gene | Locus | resistance | resistance | allele] | SEQ ID NO |
| Repaglinide | rs13266634 | SLC30A8 | Chr8: | CT + TT | CC | TGCTTCTTTATCAACAGCAG | SEQ ID NO: |
| | | | 117172544 | | | CCAGC[C/T]GGGACAGCCA | 148 |
| | | | | | | AGTGGTTCGGAGAGA | |
| | rs10494366 | NOS1AP | Chr1: | TT | TG + GG | TCAGATATTTATGGGAGGTA | SEQ ID NO: |
| | | | 162115895 | | | TGCAG[G/T]TTTTAAATTC | 184 |
| | | | | | | TGAGAATTTGTACTG | |
| | rs2237892 | KCNQ1 | Chr11: | TT | TC + CC | GGAGCTGTCACAGGACTTTG | SEQ ID NO: |
| | | | 2818521 | | | CCACC[C/T]GGGGTGAGGG | 153 |
| | | | | | | GCCTAGAAACCCCTC | |
| | rs11977021 | NAMPT | Chr7: | CT | CC + TT | ATACTCCAATCTGACCTGAT | SEQ ID NO: |
| | | | 106288069 | | | TTGAC[C/T]TCAGTAAAAA | 185 |
| | | | | | | ACACTGGTGAAGTAG | |
| | rs7651090 | IGF2BP2 | Chr3: | AG + GG | AA | GTGAATTCTTTAAGAAAATG | SEQ ID NO: |
| | | | 185795604 | | | AAGCC[A/G]GAAGCAGTGG | 164 |
| | | | | | | GTCAGTCTGTAATCC | |
| | rs5219 | KCNJ11 | Chr11: | GG | GA + AA | CGCTGGCGGGCACGGTACCT | SEQ ID NO: |
| | | | 17388025 | | | GGGCT[C/T]GGCAGGGTCC | 157 |
| | | | | | | TCTGCCAGGCGTGTC | |
| | rs7903146 | TCF7L2 | Chr10: | TT | CC + CT | TAGAGAGCTAAGCACTTTTT | SEQ ID NO: |
| | | | 112998590 | | | AGATA[C/T]TATATAATTT | 167 |
| | | | | | | AATTGCCGTATGAGG | |
| Rosiglitazone | rs13266634 | SLC30A8 | Chr8: | CC + TC | TT | TGCTTCTTTATCAACAGCAG | SEQ ID NO: |
| | | | 117172544 | | | CCAGC[C/T]GGGACAGCCA | 148 |
| | | | | | | AGTGGTTCGGAGAGA | |
| | rs2237892 | KCNQ1 | Chr11: | TT | TC + CC | GGAGCTGTCACAGGACTTTG | SEQ ID NO: |
| | | | 2818521 | | | CCACC[C/T]GGGGTGAGGG | 153 |
| | | | | | | GCCTAGAAACCCCTC | |
| | rs1801282 | PPAR-γ2 | Chr3: | CG + GG | CC | AACTCTGGGAGATTCTCCTA | SEQ ID NO: |
| | | | 12351626 | | | TTGAC[C/G]CAGAAAGCGA | 152 |
| | | | | | | TTCCTTCACTGATAC | |
| | rs6467136 | PAX4 | Chr7: | AA + AG | GG | CTTGGACATTACTTTAAAAG | SEQ ID NO: |
| | | | 127524904 | | | TGCAA[A/G]TGACAAAAGA | 159 |
| | | | | | | AAAATATAGATAAAT | |
| | rs2230806 | ABCA1 | Chr9: | GG | AA + GA | GTTTCTGAGCTTTGTGGCCT | SEQ ID NO: |
| | | | 104858586 | | | ACCAA[A/G]GGAGAAACTG | 186 |
| | | | | | | GCTGCAGCAGAGCGA | |
| Metformin | rs11212617 | near ATM | Chr11: | CC | CA + AA | CCAATTACAAAGGGCAGATC | SEQ ID NO: |
| | | | 108412434 | | | AGAGA[A/C]TGTCAGAGCG | 187 |
| | | | | | | GATAAAAAATCAAGA | |
| | rs622342 | SLC22A1 | Chr6: | AA | CC + AC | TTTCTTCAAATTTGATGAAA | SEQ ID NO: |
| | | | 160151834 | | | ACTTC[A/C]AATACATAGA | 188 |
| | | | | | | TCTAACAATCTCAAT | |
| Gliclazide | rs5219 | KCNJ11 | Chr11: | AA + GA | GG | CGCTGGCGGGCACGGTACCT | SEQ ID NO: |
| | | | 17388025 | | | GGGCT[C/T]GGCAGGGTCC | 157 |
| | | | | | | TCTGCCAGGCGTGTC | |
| Pioglitazone | rs1801282 | PPAR-γ2 | Chr3: | CG + GG | CC | AACTCTGGGAGATTCTCCTA | SEQ ID NO: |
| | | | 12351626 | | | TTGAC[C/G]CAGAAAGCGA | 152 |
| | | | | | | TTCCTTCACTGATAC | |

### II. Design of Amplification Primers and Single Base Extension Primers for SNP Combination

The amplification primers and single base extension primers in Example 1 were designed using software from the official website of sequenom.com. The sensitivity requirements of the primers are: the target fragment can be amplified in the reaction system with the DNA template as low to 10 ng. The accuracy requirements of the primers are: greater than 99.7%. The amplification primers and single base extension primers designed for the SNPs in Example 1 are shown in Table 6 and Table 7.

The primers were distributed as follows: the amplification primers for the 47 SNPs are randomly assigned to five different reaction wells (W01, W02, W03, W04, and W05), and the amplification primers for SNP with different rs numbers were assigned as shown in Table 8. After amplification primers were set in each well, the Sequenom platform was used to automatically calculate the most appropriate final reaction concentration for each primer, and use this concentration automatically.

TABLE 6
Sequences of amplification
Primer designed for the SNPs

| SNP | Primer Sequence (SEQ ID NO) | |
|---|---|---|
| rs10229583 | ACGTTGGATGCTCTTA | ACGTTGGATGGCTGCC |
| | TGTTCCAGTTTCAG | TATTCCCCACTTTG |
| | (SEQ ID NO: 1) | (SEQ ID NO: 2) |
| rs10811661 | ACGTTGGATGAGATCA | ACGTTGGATGGTCAAT |
| | GGAGGGTAATAGAC | AAGCGTTCTTGCCC |
| | (SEQ ID NO: 3) | (SEQ ID NO: 4) |
| rs10886471 | ACGTTGGATGTACAGA | ACGTTGGATGAATCTG |
| | AGTGCTTGTGTCCC | AGGATGAGCTGAAC |
| | (SEQ ID NO: 5) | (SEQ ID NO: 6) |
| rs1111875 | ACGTTGGATGAAAAAA | ACGTTGGATGACCTCC |
| | TGGACCCTGAGTGC | GTACCATCAAGTCA |
| | (SEQ ID NO: 7) | (SEQ ID NO: 8) |
| rs12742393 | ACGTTGGATGCACTAT | ACGTTGGATGAGGATA |
| | AAGCTGGGAACAGG | ACACCACCCATACC |
| | (SEQ ID NO: 9) | (SEQ ID NO: 10) |
| rs12779790 | ACGTTGGATGACCCGG | ACGTTGGATGGGGCTA |
| | ACAATGTTGGGAAT | AGGACATGAATAG |
| | (SEQ ID NO: 11) | (SEQ ID NO: 12) |
| rs13266634 | ACGTTGGATGGCAATT | ACGTTGGATGGCAATC |
| | TCTCTCCGAACCAC | AGTGCTAATCTCCC |
| | (SEQ ID NO: 13) | (SEQ ID NO: 14) |
| rs1535500 | ACGTTGGATGAGAGAT | ACGTTGGATGAGCTCT |
| | GGGGATCTTCTGAG | GGCTGCTCAGTAG |
| | (SEQ ID NO: 15) | (SEQ ID NO: 16) |
| rs16856187 | ACGTTGGATGCTCCAG | ACGTTGGATGCACACC |
| | AAGGCCATTTAGTG | GTGAATGAACCTTC |
| | (SEQ ID NO: 17) | (SEQ ID NO: 18) |
| rs16861329 | ACGTTGGATGTGTGGG | ACGTTGGATGCACTGG |
| | CCATGAATTTGAGG | TCTCTCTGCTATAC |
| | (SEQ ID NO: 19) | (SEQ ID NO: 20) |
| rs1801282 | ACGTTGGATGTGTATC | ACGTTGGATGCAAACC |
| | AGTGAAGGAATCGC | CCTATTCCATGCTG |
| | (SEQ ID NO: 21) | (SEQ ID NO: 22) |
| rs2237892 | ACGTTGGATGCAGATG | ACGTTGGATGTGTAAG |
| | ATGGGAGCTGTCAC | GCATCTGGTGGAGA |
| | (SEQ ID NO: 23) | (SEQ ID NO: 24) |
| rs340874 | ACGTTGGATGCATATA | ACGTTGGATGGAGCAG |
| | AGTTAGCGCCAGCC | ATGGTTTTAAGGTG |
| | (SEQ ID NO: 25) | (SEQ ID NO: 26) |
| rs3786897 | ACGTTGGATGTCATCT | ACGTTGGATGAAGCCA |
| | GCATAGGACAGCCC | TCCTGGAAGACCTG |
| | (SEQ ID NO: 27) | (SEQ ID NO: 28) |
| rs4430796 | ACGTTGGATGCAAAGA | ACGTTGGATGTGAATA |
| | CCCAACAACGCTTG | CAGAGAGGCAGCAC |
| | (SEQ ID NO: 29) | (SEQ ID NO: 30) |
| rs5219 | ACGTTGGATGGGCATC | ACGTTGGATGTCCGCT |
| | ATCCCCGAGGAATA | GGCGGGCACGGTA |
| | (SEQ ID NO: 31) | (SEQ ID NO: 32) |
| rs6017317 | ACGTTGGATGAAGAAA | ACGTTGGATGCTGTTG |
| | AGGCAAAGGATCTG | GCCATTTCTATGTTG |
| | (SEQ ID NO: 33) | (SEQ ID NO: 34) |
| rs6467136 | ACGTTGGATGTGATGA | ACGTTGGATGAGGTAA |
| | GATCCAATTTATC | TATTTTCTTGGAC |
| | (SEQ ID NO: 35) | (SEQ ID NO: 36) |
| rs6815464 | ACGTTGGATGGCAAAG | ACGTTGGATGTCTGCA |
| | CTCTCACGAGGAAG | CACATCCTGCTTAG |
| | (SEQ ID NO: 37) | (SEQ ID NO: 38) |
| rs7041847 | ACGTTGGATGGATGCC | ACGTTGGATGTTTACC |
| | GCGTTGTAAATCAC | ACCTCATCGCATAC |
| | (SEQ ID NO: 39) | (SEQ ID NO: 40) |
| rs7172432 | ACGTTGGATGCTTTGG | ACGTTGGATGCTGGCT |
| | GAGATAGGTTCTGC | TAAAAGAGGGCTTG |
| | (SEQ ID NO: 41) | (SEQ ID NO: 42) |
| rs7612463 | ACGTTGGATGCTGCCT | ACGTTGGATGAGAGAA |
| | AATACAGGGTCTTC | GTGGTTTTAGACTC |
| | (SEQ ID NO: 43) | (SEQ ID NO: 44) |
| rs7651090 | ACGTTGGATGAGGAGA | ACGTTGGATGTGCTGG |
| | CAAAATCATCTGGG | GATTACAGACTGAC |
| | (SEQ ID NO: 45) | (SEQ ID NO: 46) |
| rs7756992 | ACGTTGGATGGCAAAA | ACGTTGGATGGACAAT |
| | GGACTGATAATGAGC | TAATATTCCCCCCTG |
| | (SEQ ID NO: 47) | (SEQ ID NO: 48) |
| rs780094 | ACGTTGGATGCCCGGC | ACGTTGGATGAGGGCC |
| | CTCAACAAATGTAT | CCAGTTTTTTAGAC |
| | (SEQ ID NO: 49) | (SEQ ID NO: 50) |
| rs7903146 | ACGTTGGATGAACTAA | ACGTTGGATGACAATT |
| | GGGTGCCTCATACG | AGAGAGCTAAGCAC |
| | (SEQ ID NO: 51) | (SEQ ID NO: 52) |
| rs8050136 | ACGTTGGATGCTCTAC | ACGTTGGATGTAGTCT |
| | AGTTTACCTAAGGC | AGGCATGCCAGTTG |
| | (SEQ ID NO: 53) | (SEQ ID NO: 54) |
| rs831571 | ACGTTGGATGAGCTGG | ACGTTGGATGGACAAG |
| | TGACCTAGAGATAG | GCTATCCATCCTC |
| | (SEQ ID NO: 55) | (SEQ ID NO: 56) |
| rs9470794 | ACGTTGGATGGCTGGG | ACGTTGGATGCTTCTG |
| | TGATTTTAGAGGAG | TGATGTCACACTCC |
| | (SEQ ID NO: 57) | (SEQ ID NO: 58) |
| rs2268388 | ACGTTGGATGGAGAAA | ACGTTGGATGGTGCTG |
| | AGCCTGCAGGGCTA | TTCTCCCAGCAGA |
| | (SEQ ID NO: 59) | (SEQ ID NO: 60) |
| rs9565164 | ACGTTGGATGGATTCC | ACGTTGGATGATTTTG |
| | AGAGTCCAGGAAC | CAGAGGAGTGGGAC |
| | (SEQ ID NO: 61) | (SEQ ID NO: 62) |
| rs4668142 | ACGTTGGATGATCATT | ACGTTGGATGTCTCTC |
| | CTGGGGTAATAGGC | TCCACTCAATCTCG |
| | (SEQ ID NO: 63) | (SEQ ID NO: 64) |
| rs238021 | ACGTTGGATGTGTTGG | ACGTTGGATGAACAAG |
| | GAAGGTTTAGATGC | GTAGCCCAGCCAAG |
| | (SEQ ID NO: 65) | (SEQ ID NO: 66) |
| rs39059 | ACGTTGGATGAGGACC | ACGTTGGATGTCAGAA |
| | ATAACCTATGGGAC | TCAGTCACTTAAGG |
| | (SEQ ID NO: 67) | (SEQ ID NO: 68) |
| rs17756941 | ACGTTGGATGGAATGA | ACGTTGGATGCCTTTT |
| | AGTATTGGTGTGTGC | GGAATGTGACCTAC |
| | (SEQ ID NO: 69) | (SEQ ID NO: 70) |
| rs245955 | ACGTTGGATGCCCATT | ACGTTGGATGGTTTCT |
| | TGAGAACTCTGACC | TGTTGCTGCTATAA |
| | (SEQ ID NO: 71) | (SEQ ID NO: 72) |
| rs245962 | ACGTTGGATGGTTCCT | ACGTTGGATGGAAGGT |
| | AGTCTAAAATAGTC | ACAGAAGACATGAG |
| | (SEQ ID NO: 73) | (SEQ ID NO: 74) |
| rs266729 | ACGTTGGATGATGTGT | ACGTTGGATGACCTTG |
| | GGCTTGCAAGAACC | GACTTTCTTGGCAC |
| | (SEQ ID NO: 75) | (SEQ ID NO: 76) |
| rs3811951 | ACGTTGGATGTTTCCT | ACGTTGGATGGCTGAT |
| | ACCCCAAACACATC | TCATGAGTACTCTC |
| | (SEQ ID NO: 77) | (SEQ ID NO: 78) |
| rs156019 | ACGTTGGATGAGCAGA | ACGTTGGATGCCTGCT |
| | AGGCAACACGTTTC | GCTTTTGGGTTTTT |
| | (SEQ ID NO: 79) | (SEQ ID NO: 80) |
| rs6234 | ACGTTGGATGATGAGT | ACGTTGGATGCTTTGG |
| | TGGAGGAGGGAGC | CGGTGAGTTTTTAC |
| | (SEQ ID NO: 81) | (SEQ ID NO: 82) |
| rs3856806 | ACGTTGGATGGCTGCT | ACGTTGGATGTCTCCG |
| | CCAGAAAATGACAG | TCTTCTTGATCACC |
| | (SEQ ID NO: 83) | (SEQ ID NO: 84) |
| rs10494366 | ACGTTGGATGGTGTCC | ACGTTGGATGGATCAG |
| | TAGATAGAGACCAG | ATATTTATGGGAGG |
| | (SEQ ID NO: 85) | (SEQ ID NO: 86) |
| rs11977021 | ACGTTGGATGAAAGTT | ACGTTGGATGCAGGTA |
| | GCCTCAGATACTCC | GGTGAGTTCCATAC |
| | (SEQ ID NO: 87) | (SEQ ID NO: 88) |
| rs2230806 | ACGTTGGATGTCAACT | ACGTTGGATGCAGGAT |
| | TGGTGACCAAGAAG | GTCCATGTTGGAAC |
| | (SEQ ID NO: 89) | (SEQ ID NO: 90) |
| rs11212617 | ACGTTGGATGACCAAT | ACGTTGGATGGTGGGT |
| | TACAAAGGGCAGAT | TGGTTGTGGATAAC |
| | (SEQ ID NO: 91) | (SEQ ID NO: 92) |
| rs622342 | ACGTTGGATGTAGGAG | ACGTTGGATGGGTTAG |
| | GGGTTAATAGAGAG | TATTTATTGAGATTG |
| | (SEQ ID NO: 93) | (SEQ ID NO: 91) |

TABLE 7
Primer sequences for detecting
SNPs by single base extension

| SNP | Primer Sequence | SEQ ID NO |
|---|---|---|
| rs10229583 | TGATTTTTAAATCTGTTGACAA | SEQ ID NO: 95 |
| rs10811661 | ggCACCTCCAGCTTTAGTTTTC | SEQ ID NO: 96 |
| rs10886471 | AAAAGCAGCTGGGCCAAGA | SEQ ID NO: 97 |
| rs1111875 | CCATCAAGTCATTTCCTCT | SEQ ID NO: 98 |
| rs12742393 | CCACCCATACCACAGATTGTAC | SEQ ID NO: 99 |
| rs12779790 | tgAGATATATAAATGGCCAAGAA | SEQ ID NO: 100 |
| | ATA | |
| rs13266634 | TATCAACAGCAGCCAGC | SEQ ID NO: 101 |
| rs1535500 | CTCCTCGGCGTCAAGGATGGGG | SEQ ID NO: 102 |
| rs16856187 | cAACCCCATACTAAACTCC | SEQ ID NO: 103 |
| rs16861329 | TCTCTGCTATACCAACAC | SEQ ID NO: 104 |
| rs1801282 | AAACTCTGGGAGATTCTCCTATT | SEQ ID NO: 105 |
| | GAC | |
| rs2237892 | TCTAGGCCCCTCACCCC | SEQ ID NO: 106 |
| rs340874 | cAAGGTGTGGAAAGGTATA | SEQ ID NO: 107 |
| rs3786897 | TGTCTTCCAGGGAAAAAGGG | SEQ ID NO: 108 |
| rs4430796 | AGGCAGCACAGACTGGA | SEQ ID NO: 109 |
| rs5219 | GGCACGGTACCTGGGCT | SEQ ID NO: 110 |
| rs6017317 | TCTATGTTGTCTTGTTTTGAG | SEQ ID NO: 111 |
| rs6467136 | ggagGGACATTACTTTAAAAGTG | SEQ ID NO: 112 |
| | CAA | |
| rs6815464 | ctATACCTGTACCCCGGGTTTTG | SEQ ID NO: 113 |
| rs7041847 | CTCATCGCATACATTTTGGGGAG | SEQ ID NO: 114 |
| | CTA | |
| rs7172432 | cAGGGCCCACTACAGTTTCTTTG | SEQ ID NO: 115 |
| | GGAA | |
| rs7612463 | GGTTTTAGACTCAGTTTCAGGTA | SEQ ID NO: 116 |
| rs7651090 | GACTGACCCACTGCTTC | SEQ ID NO: 117 |
| rs7756992 | cCCCCCCTGTATTTTAGTTTTT | SEQ ID NO: 118 |
| rs780094 | ggTTAGACCATGACTGACACAT | SEQ ID NO: 119 |
| rs7903146 | GAGCTAAGCACTTTTTAGATA | SEQ ID NO: 120 |
| rs8050136 | TGCCAGTTGCCCACTGTGGCAAT | SEQ ID NO: 121 |
| rs831571 | aCTCTTGACAACAAGATAGGCTT | SEQ ID NO: 122 |
| | TA | |
| rs9470794 | aTCCTTTCTTCCCACCTGACC | SEQ ID NO: 123 |
| rs2268388 | taTGTTCTCCCAGCAGAGAACAC | SEQ ID NO: 124 |
| | TC | |
| rs9565164 | GGAGTGGGACAAAACTA | SEQ ID NO: 125 |
| rs4668142 | TGCCAGCCAATGTTCTGA | SEQ ID NO: 126 |
| rs2380261 | GCCCAGCCAAGCATGGCTCCAAG | SEQ ID NO: 127 |
| | G | |
| rs39059 | aaagCAGTCACTTAAGGAAGAAG | SEQ ID NO: 128 |
| | AG | |
| rs17756941 | gagGGAATGTGACCTACACTATG | SEQ ID NO: 129 |
| rs245955 | TAATAGCTAACATAATACAAAGT | SEQ ID NO: 130 |
| | TAC | |
| rs245962 | ACATGAGACTTTCATGAATTATA | SEQ ID NO: 131 |
| rs266729 | GGCACGCTCATGTTTTGTTTTTG | SEQ ID NO: 132 |
| | AAG | |
| rs3811951 | TCATGAGTACTCTCACATTAACA | SEQ ID NO: 133 |
| | A | |
| rs156019 | ggACACTAGTAAAAAACTACTCA | SEQ ID NO: 134 |
| | G | |
| rs6234 | GAGTCGCAGCATGGCCT | SEQ ID NO: 135 |
| rs3856806 | gcTCACCTGCAGTAGCTGCAC | SEQ ID NO: 136 |
| rs10494366 | TTATGGGAGGTATGCAG | SEQ ID NO: 137 |
| rs11977021 | CACCAGTGTTTTTTACTGA | SEQ ID NO: 138 |
| rs2230806 | TGCTGCAGCCAGTTTCTCC | SEQ ID NO: 139 |
| rs11212617 | TTTTTTATCCGCTCTGACA | SEQ ID NO: 140 |
| rs622342 | TTGAGATTGTTAGATCTATGTAT | SEQ ID NO: 141 |
| | T | |

TABLE 8
Primers for SNP with different rs are assigned in different wells.

| SNP | Well |
|---|---|
| rs10229583 | W02 |
| rs10494366 | W04 |
| rs10811661 | W01 |
| rs10886471 | W02 |
| rs1111875 | W03 |
| rs11212617 | W01 |
| rs11977021 | W02 |
| rs12742393 | W01 |
| rs12779790 | W02 |
| rs13266634 | W03 |
| rs1535500 | W04 |
| rs156019 | W03 |
| rs16856187 | W01 |
| rs16861329 | W03 |
| rs17756941 | W02 |
| rs1801282 | W03 |
| rs2230806 | W05 |
| rs2237892 | W01 |
| rs2268388 | W03 |
| rs2380261 | W01 |
| rs245955 | W03 |
| rs245962 | W02 |
| rs266729 | W03 |
| rs340874 | W01 |
| rs3786897 | W02 |
| rs3811951 | W03 |
| rs3856806 | W01 |
| rs39059 | W01 |
| rs4430796 | W04 |
| rs4668142 | W02 |
| rs5219 | W04 |
| rs6017317 | W05 |
| rs622342 | W02 |
| rs6234 | W03 |
| rs6467136 | W03 |
| rs6815464 | W03 |
| rs7041847 | W02 |
| rs7172432 | W01 |
| rs7612463 | W03 |
| rs7651090 | W02 |
| rs7756992 | W01 |
| rs780094 | W02 |
| rs7903146 | W02 |
| rs8050136 | W01 |
| rs831571 | W01 |
| rs9470794 | W02 |
| rs9565164 | W02 |

The amplification primers and single base extension primer may also be part of a kit to detect the following SNPs: rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342.

### Example 2

### Application of the Amplification Primers and Single Base Extension Primer in the Detection of SNPs Associated with Diabetes

I. Blood Sample Collection:

3252 blood samples of diabetes patients from Shanghai Sixth People's Hospital were collected. All patients were diagnosed clinically and the DNA sequences were confirmed by sequencing. Informed consent was obtained from each participant. 3359 blood samples of normal people were also collected.

II. DNA Extraction:

DNA was extracted from blood samples obtained in Step 1, as follows: adding 250 μl blood and 225 μl GH buffer into a centrifuge tube with 1.5 ml protease K; mixing by pipetting or shaking; incubating for 10 min at 65° C.; mixing for three times during the incubation by inversion or vibration; adding 20 μl bead suspension G; mixing by pipetting or shaking; adding 325 μl isopropanol (alternatively, beads can be first mixed with isopropanol in an ratio as above and then the mixture can be added together); mixing by pipetting or shaking for 15 min; removing the liquid carefully when beads were completely adsorbed after standing for 30 seconds on a magnetic rack stand and then adding 700 μl wash buffer I; mixing by pipetting or shaking; removing the liquid carefully when beads were completely adsorbed after standing for 30 seconds on a magnetic rack stand and then adding 700 μl wash buffer II; mixing by pipetting or shaking; letting the centrifuge tube dry on the magnetic stand at room temperature for 10 minutes after removing the liquid carefully when beads were completely adsorbed after standing for 30 seconds on a magnetic rack stand; adding 6510 eluent TB to the centrifuge tube after it was removed from the magnetic rack stand; mixing by pipetting or shaking; incubating for 10 minutes at 56° C.; mixing for three times during the incubation by inversion or vibration (the speed can also be adjusted to 1500 rpm if thermostatic bath with the function of oscillation is used). The DNA solution in the centrifuge tube was transferred to a collection plate when beads were completely adsorbed after standing for 2 minutes on the magnetic rack stand. The DNA solution was stored at −20° C.

III. PCR Amplification:

Diluted DNA derived from the blood samples extracted from Step II was added to the 384-well plate, and multiplex PCR amplification was performed as described below. The multiplex PCR amplification reaction system was added to each well, including: 25 mM dNTPs 0.1 μl, 10×PCR Buffer 0.4 μl, 25 mmol/L MgCl₂ 0.4 μl, template DNA 10 ng, 0.5 μM primer mixture (amplification primers of the all SNP loci added to each well as shown in Table 8) 1 μl, HotstarTaq (5 U/μl) 0.1 μl. The reaction volume was brought up to 5 μl by water.

The 384-well plate was sealed by the film to prevent sample evaporation. The sealed plate was placed on AB19700 PCR instrument, with the following conditions: initial denaturation at 94° C. 2 min; 94° C. 20 sec; 56° C. 30 sec; 72° C. 1 min; 45 cycles; 72° C. 3 min; hold at 4° C. The resulting PCR products were centrifuged at 2500 rpm for Imin.

The reagents in the following steps IV-VIII were obtained from Sequenom for use with the Sequenom platform, and results were obtained according to the manufacturer's instructions. The specific steps are as follows.

IV. Digestion by SAP:

The PCR amplification products obtained from Step III were digested by SAP (shrimp alkaline phosphatase), after being centrifuged at 2000 rpm for 1 min. The specific steps were as follows: preparing the SAP reaction mix (2 μl/well) that is 110% of the required sample volume: SAP Buffer 0.17 μl, SAP Enzyme 0.3 μl, and the total reaction volume was brought up to 2 μl by water.

The SAP reaction mix (2 μl/well) was then added to the 384-well plate after Step III, and the reaction mix was pipetted for five times with a multi-channel pipettor. The plate was sealed with a film membrane, vortexed for 30 seconds and centrifuged at 2500 rpm for 1 min.

The 384-plate was placed on the AB19700 PCR instrument and was set with the following reaction procedure: 37° C. 40 min, 85° C. 5 min, hold at 4° C., to obtain the SAP digested product. The SAP digested product was centrifuged at 2500 rpm for 1 min.

V. Single Base Extension Reaction:

The SAP digested products obtained from Step IV were subject to single base extension reaction. The specific steps were as follows: preparing the extension mix with 20% redundancy (i.e., at a volume of 120% of the required volume), which contained iPLEX Buffer plus 0.2 μl, iPLEX Terminator 0.2 μl, iPLEX Enzyme 0.041 μl, primer mix 1 μl (single base extension primers of the all SNP loci added to each well as shown in Table 8). The total volume was brought up to 2.06 μl by water. The extension mix (2.06 μl) was added to the 384-well plate after Step IV by a multi-channel pipettor, and was pipetted for five times. The plate was sealed with a film membrane, and centrifuged at 2500 rpm for 1 min. The 384-plate was then placed on the ABI9700 PCR instrument and the extension reaction procedure was set as shown in Table 9. Then the single base extension reaction products were centrifuged at 2500 rpm for 1 min.

TABLE 9
Procedures of single base extension reaction

| | | | | | |
|---|---|---|---|---|---|
| First | 94° C. | 30 | s | | |
| Second | 94° C. | 5 | s | 1 cycle | 40 cycles |
| | 52° C. | 5 | s | 5 cycles | |
| | 80° C. | 5 | s | | |
| Third | 72° C. | 3 | min | | |

| | | |
|---|---|---|
| Forth | 4° C. | ∞ |

VI. Resin Purification:

The single base extension reaction products from Step V were centrifuged at 3000 rpm for 2 min. 16 μl MBG water was added to each well. The 384-well plate was sealed with a film, and then centrifuged at 3000 rpm for 1 min. A 6 mg plate was placed on a clean sheet of A4 paper, and an appropriate amount of resin was placed on the 384-well plate with a spoon. A plastic lid was then used to compact the resin several times so that each well contained an equal amount of the resin. The 384-well plate was faced to the 6 mg plate well-to-well. The 6 mg plate was on the top and the 384-well plate was on the bottom. The back of the 6 mg plate was taped gently so that the resin fell into the wells of 384-well plate equipped with the single-base extension product. The 384-well plate was sealed with a film and rotated vertically for 30 min by a low-speed shaker to mix the resin and reagents. The plate was then centrifuged at 3000 rpm for 3 min to collect the resin to the bottom of the wells and the purified extension products were obtained.

VII. Mass Spectrometry Detection:

First, the MassARRAY Nanodispenser RS 1000 spotter was used to spot the resin purified extension products from Step VI to the chip of 384 dot SpectroCHIP (Sequenom) to obtain the spotted SpectroCHIP chip. Then MALDI-TOF (matrix-assisted laser desorption/ionization-time of flight) was used to analyze the spotted SpectroCHIP chip, and TYPER 4.0 software (Sequenom) was used to obtain the genotyping results.

VIII. The Detection Results:

The results suggest that the SNP genotyping results of 47 SNP sites for all samples are consistent with the results from DNA sequencing. The SNP genotyping results of one test sample (test sample 1 of diabetic patients) are shown in Table 10. The amplification primers of the present invention and a single-base primer extension can be effectively used to detect the following SNP loci: rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and s622342.

TABLE 10
Genotyping results of SNP sites

| SNPs | Genotyping results |
|---|---|
| rs10229583 | G |
| rs10494366 | GT |
| rs10811661 | CT |
| rs10886471 | C |
| rs1111875 | A |
| rs11212617 | C |
| rs11977021 | C |
| rs12742393 | CA |
| rs12779790 | A |
| rs13266634 | T |
| rs1535500 | GT |
| rs156019 | TA |
| rs16856187 | CA |
| rs16861329 | CT |
| rs17756941 | A |
| rs1801282 | C |
| rs2230806 | GA |
| rs2237892 | TC |
| rs2268388 | C |
| rs2380261 | T |
| rs245955 | CT |
| rs245962 | AG |
| rs266729 | C |
| rs340874 | A |
| rs3786897 | AG |
| rs3811951 | A |
| rs3856806 | C |
| rs39059 | GA |
| rs4430796 | A |
| rs4668142 | GT |
| rs5219 | CT |
| rs6017317 | G |
| rs622342 | A |
| rs6234 | C |
| rs6467136 | G |
| rs6815464 | CG |
| rs7041847 | GA |
| rs7172432 | A |
| rs7612463 | CA |
| rs7651090 | A |
| rs7756992 | G |
| rs780094 | G |
| rs7903146 | CT |
| rs8050136 | C |
| rs831571 | CT |
| rs9470794 | TC |
| rs9565164 | T |

Various embodiments in the device of the present disclosure are described in a progressive manner. Differences between various embodiments are emphasized in their specifications, while their common structures can be referred from the description.

### Embodiment 1

A primer set for detecting an SNP (Single Nucleotide Polymorphism) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, wherein the primers in the set are selected from the group consisting of Primer 1 to Primer 47.

wherein Primer 1 comprises at least one amplification primer for detecting rs10229583, Primer 2 comprises at least one amplification primer for detecting rs10811661; Primer 3 comprises at least one amplification primer for detecting rs10886471; Primer 4 comprises at least one amplification primer for detecting rs1111875; Primer 5 comprises at least one amplification primer for detecting rs12742393, Primer 6 comprises at least one amplification primer for detecting rs12779790; Primer 7 comprises at least one amplification primer for detecting rs13266634; Primer 8 comprises at least one amplification primer for detecting rs1535500; Primer 9 comprises at least one amplification primer for detecting rs16856187; Primer 10 comprises at least one amplification primer for detecting rs16861329; Primer 11 comprises at least one amplification primer for detecting rs1801282; Primer 12 comprises at least one amplification primer for detecting rs2237892; Primer 13 comprises at least one amplification primer for detecting rs340874; Primer 14 comprises at least one amplification primer for detecting rs3786897; Primer 15 comprises at least one amplification primer for detecting rs4430796; Primer 16 comprises at least one amplification primer for detecting rs5219; Primer 17 comprises at least one amplification primer for detecting rs6017317; Primer 18 comprises at least one amplification primer for detecting rs6467136; Primer 19 comprises at least one amplification primer for detecting rs6815464; Primer 20 comprises at least one amplification primer for detecting rs7041847; Primer 21 comprises at least one amplification primer for detecting rs7172432; Primer 22 comprises at least one amplification primer for detecting rs7612463; Primer 23 comprises at least one amplification primer for detecting rs7651090; Primer 24 comprises at least one amplification primer for detecting rs7756992; Primer 25 comprises at least one amplification primer for detecting rs780094; Primer 26 comprises at least one amplification primer for detecting rs7903146; Primer 27 comprises at least one amplification primer for detecting rs8050136; Primer 28 comprises at least one amplification primer for detecting rs831571; Primer 29 comprises at least one amplification primer for detecting rs9470794; Primer 30 comprises at least one amplification primer for detecting rs2268388; Primer 31 comprises at least one amplification primer for detecting rs9565164; Primer 32 comprises at least one amplification primer for detecting rs4668142; Primer 33 comprises at least one amplification primer for detecting rs2380261; Primer 34 comprises at least one amplification primer for detecting rs39059; Primer 35 comprises at least one amplification primer for detecting rs17756941; Primer 36 comprises at least one amplification primer for detecting rs245955; Primer 37 comprises at least one amplification primer for detecting rs245962; Primer 38 comprises at least one amplification primer for detecting rs266729; Primer 39 comprises at least one amplification primer for detecting rs3811951; Primer 40 comprises at least one amplification primer for detecting rs156019; Primer 41 comprises at least one amplification primer for detecting rs6234; Primer 42 comprises at least one amplification primer for detecting rs3856806; Primer 43 comprises at least one amplification primer for detecting rs10494366; Primer 44 comprises at least one amplification primer for detecting rs11977021; Primer 45 comprises at least one amplification primer for detecting rs2230806; Primer 46 comprises at least one amplification primer for detecting rs11212617; and/or Primer 47 comprises at least one amplification primer for detecting rs622342,

optionally wherein the at least one amplification primer for detecting rs10229583 comprises the single-chain DNA sequence set forth in SEQ ID NO: 1 and/or the single-chain DNA sequence set forth in SEQ ID NO: 2; the at least one amplification primer for detecting rs10811661 comprises the single-chain DNA sequence set forth in SEQ ID NO: 3 and/or the single-chain DNA sequence set forth in SEQ ID NO: 4; the at least one amplification primer for detecting rs10886471 comprises the single-chain DNA sequence set forth in SEQ ID NO: 5 and/or the single-chain DNA sequence set forth in SEQ ID NO: 6; the at least one amplification primer for detecting rs1111875 comprises the single-chain DNA sequence set forth in SEQ ID NO: 7 and/or the single-chain DNA sequence set forth in SEQ ID NO: 8; the at least one amplification primer for detecting rs12742393 comprises the single-chain DNA sequence set forth in SEQ ID NO: 9 and/or the single-chain DNA sequence set forth in SEQ ID NO: 10; the at least one amplification primer for detecting rs12779790 comprises the single-chain DNA sequence set forth in SEQ ID NO: 11 and/or the single-chain DNA sequence set forth in SEQ ID NO: 12; the at least one amplification primer for detecting rs13266634 comprises the single-chain DNA sequence set forth in SEQ ID NO: 13 and/or the single-chain DNA sequence set forth in SEQ ID NO: 14; the at least one amplification primer for detecting rs1535500 comprises the single-chain DNA sequence set forth in SEQ ID NO: 15 and/or the single-chain DNA sequence set forth in SEQ ID NO: 16; the at least one amplification primer for detecting rs16856187 comprises the single-chain DNA sequence set forth in SEQ ID NO: 17 and/or the single-chain DNA sequence set forth in SEQ ID NO: 18; the at least one amplification primer for detecting rs16861329 comprises the single-chain DNA sequence set forth in SEQ ID NO: 19 and/or the single-chain DNA sequence set forth in SEQ ID NO: 20; the at least one amplification primer for detecting rs1801282 comprises the single-chain DNA sequence set forth in SEQ ID NO: 21 and/or the single-chain DNA sequence set forth in SEQ ID NO: 22; the at least one amplification primer for detecting rs2237892 comprises the single-chain DNA sequence set forth in SEQ ID NO: 23 and/or the single-chain DNA sequence set forth in SEQ ID NO: 24; the at least one amplification primer for detecting rs340874 comprises the single-chain DNA sequence set forth in SEQ ID NO: 25 and/or the single-chain DNA sequence set forth in SEQ ID NO: 26; the at least one amplification primer for detecting rs3786897 comprises the single-chain DNA sequence set forth in SEQ ID NO: 27 and/or the single-chain DNA sequence set forth in SEQ ID NO: 28; the at least one amplification primer for detecting rs4430796 comprises the single-chain DNA sequence set forth in SEQ ID NO: 29 and/or the single-chain DNA sequence set forth in SEQ ID NO: 30; the at least one amplification primer for detecting rs5219 comprises the single-chain DNA sequence set forth in SEQ ID NO: 31 and/or the single-chain DNA sequence set forth in SEQ ID NO: 32; the at least one amplification primer for detecting rs6017317 comprises the single-chain DNA sequence set forth in SEQ ID NO: 33 and/or the single-chain DNA sequence set forth in SEQ ID NO: 34; the at least one amplification primer for detecting rs6467136 comprises the single-chain DNA sequence set forth in SEQ ID NO: 35 and/or the single-chain DNA sequence set forth in SEQ ID NO: 36; the at least one amplification primer for detecting rs6815464 comprises the single-chain DNA sequence set forth in SEQ ID NO: 37 and/or the single-chain DNA sequence set forth in SEQ ID NO: 38; the at least one amplification primer for detecting rs7041847 comprises the single-chain DNA sequence set forth in SEQ ID NO: 39 and/or the single-chain DNA sequence set forth in SEQ ID NO: 40; the at least one amplification primer for detecting rs7172432 comprises the single-chain DNA sequence set forth in SEQ ID NO: 41 and/or the single-chain DNA sequence set forth in SEQ ID NO: 42; the at least one amplification primer for detecting rs7612463 comprises the single-chain DNA sequence set forth in SEQ ID NO: 43 and/or the single-chain DNA sequence set forth in SEQ ID NO: 44; the at least one amplification primer for detecting rs7651090 comprises the single-chain DNA sequence set forth in SEQ ID NO: 45 and/or the single-chain DNA sequence set forth in SEQ ID NO: 46; the at least one amplification primer for detecting rs7756992 comprises the single-chain DNA sequence set forth in SEQ ID NO: 47 and/or the single-chain DNA sequence set forth in SEQ ID NO: 48; the at least one amplification primer for detecting rs780094 comprises the single-chain DNA sequence set forth in SEQ ID NO: 49 and/or the single-chain DNA sequence set forth in SEQ ID NO: 50; the at least one amplification primer for detecting rs7903146 comprises the single-chain DNA sequence set forth in SEQ ID NO: 51 and/or the single-chain DNA sequence set forth in SEQ ID NO: 52; the at least one amplification primer for detecting rs8050136 comprises the single-chain DNA sequence set forth in SEQ ID NO: 53 and/or the single-chain DNA sequence set forth in SEQ ID NO: 54; the at least one amplification primer for detecting rs831571 comprises the single-chain DNA sequence set forth in SEQ ID NO: 55 and/or the single-chain DNA sequence set forth in SEQ ID NO: 56; the at least one amplification primer for detecting rs9470794 comprises the single-chain DNA sequence set forth in SEQ ID NO: 57 and/or the single-chain DNA sequence set forth in SEQ ID NO: 58; the at least one amplification primer for detecting rs2268388 comprises the single-chain DNA sequence set forth in SEQ ID NO: 59 and/or the single-chain DNA sequence set forth in SEQ ID NO: 60; the at least one amplification primer for detecting rs9565164 comprises the single-chain DNA sequence set forth in SEQ ID NO: 61 and/or the single-chain DNA sequence set forth in SEQ ID NO: 62; the at least one amplification primer for detecting rs4668142 comprises the single-chain DNA sequence set forth in SEQ ID NO: 63 and/or the single-chain DNA sequence set forth in SEQ ID NO: 64; the at least one amplification primer for detecting rs2380261 comprises the single-chain DNA sequence set forth in SEQ ID NO: 65 and/or the single-chain DNA sequence set forth in SEQ ID NO: 66; the at least one amplification primer for detecting rs39059 comprises the single-chain DNA sequence set forth in SEQ ID NO: 67 and/or the single-chain DNA sequence set forth in SEQ ID NO: 68; the at least one amplification primer for detecting rs17756941 comprises the single-chain DNA sequence set forth in SEQ ID NO: 69 and/or the single-chain DNA sequence set forth in SEQ ID NO: 70; the at least one amplification primer for detecting rs245955 comprises the single-chain DNA sequence set forth in SEQ ID NO: 71 and/or the single-chain DNA sequence set forth in SEQ ID NO: 72; the at least one amplification primer for detecting rs245962 comprises the single-chain DNA sequence set forth in SEQ ID NO: 73 and/or the single-chain DNA sequence set forth in SEQ ID NO: 74; the at least one amplification primer for detecting rs266729 comprises the single-chain DNA sequence set forth in SEQ ID NO: 75 and/or the single-chain DNA sequence set forth in SEQ ID NO: 76; the at least one amplification primer for detecting rs3811951 comprises the single-chain DNA sequence set forth in SEQ ID NO: 77 and/or the single-chain DNA sequence set forth in SEQ ID NO: 78; the at least one amplification primer for detecting rs156019 comprises the single-chain DNA sequence set forth in SEQ ID NO: 79 and/or the single-chain DNA sequence set forth in SEQ ID NO: 80; the at least one amplification primer for detecting rs6234 comprises the single-chain DNA sequence set forth in SEQ ID NO: 81 and/or the single-chain DNA sequence set forth in SEQ ID NO: 82; the at least one amplification primer for detecting rs3856806 comprises the single-chain DNA sequence set forth in SEQ ID NO: 83 and/or the single-chain DNA sequence set forth in SEQ ID NO: 84; the at least one amplification primer for detecting rs10494366 comprises the single-chain DNA sequence set forth in SEQ ID NO: 85 and/or the single-chain DNA sequence set forth in SEQ ID NO: 86; the at least one amplification primer for detecting rs11977021 comprises the single-chain DNA sequence set forth in SEQ ID NO: 87 and/or the single-chain DNA sequence set forth in SEQ ID NO: 88; the at least one amplification primer for detecting rs2230806 comprises the single-chain DNA sequence set forth in SEQ ID NO: 89 and/or the single-chain DNA sequence set forth in SEQ ID NO: 90; the at least one amplification primer for detecting rs11212617 comprises the single-chain DNA sequence set forth in SEQ ID NO: 91 and/or the single-chain DNA sequence set forth in SEQ ID NO: 92; and/or the at least one amplification primer for detecting rs622342 comprises the single-chain DNA sequence set forth in SEQ ID NO: 93 and/or the single-chain DNA sequence set forth in SEQ ID NO: 94.

### Embodiment 2

The primer set of Embodiment 1, wherein Primer 1 further comprises a primer for detecting rs10229583 by single base extension; Primer 2 further comprises a primer for detecting rs10811661 by single base extension; Primer 3 further comprises a primer for detecting rs10886471 by single base extension; Primer 4 further comprises a primer for detecting rs1111875 by single base extension; Primer 5 further comprises a primer for detecting rs12742393 by single base extension; Primer 6 further comprises a primer for detecting rs12779790 by single base extension; Primer 7 further comprises a primer for detecting rs13266634 by single base extension; Primer 8 further comprises a primer for detecting rs1535500 by single base extension; Primer 9 further comprises a primer for detecting rs16856187 by single base extension; Primer 10 further comprises a primer for detecting rs16861329 by single base extension; Primer 11 further comprises a primer for detecting rs1801282 by single base extension; Primer 12 further comprises a primer for detecting rs2237892 by single base extension; Primer 13 further comprises a primer for detecting rs340874 by single base extension; Primer 14 further comprises a primer for detecting rs3786897 by single base extension; Primer 15 further comprises a primer for detecting rs4430796 by single base extension; Primer 16 further comprises a primer for detecting rs5219 by single base extension; Primer 17 further comprises a primer for detecting rs6017317 by single base extension; Primer 18 further comprises a primer for detecting rs6467136 by single base extension; Primer 19 further comprises a primer for detecting rs6815464 by single base extension; Primer 20 further comprises a primer for detecting rs7041847 by single base extension; Primer 21 further comprises a primer for detecting rs7172432 by single base extension; Primer 22 further comprises a primer for detecting rs7612463 by single base extension; Primer 23 further comprises a primer for detecting rs7651090 by single base extension; Primer 24 further comprises a primer for detecting rs7756992 by single base extension; Primer 25 further comprises a primer for detecting rs780094 by single base extension; Primer 26 further comprises a primer for detecting rs7903146 by single base extension; Primer 27 further comprises a primer for detecting rs8050136 by single base extension; Primer 28 further comprises a primer for detecting rs831571 by single base extension; Primer 29 further comprises a primer for detecting rs9470794 by single base extension; Primer 30 further comprises a primer for detecting rs2268388 by single base extension; Primer 31 further comprises a primer for detecting rs9565164 by single base extension; Primer 32 further comprises a primer for detecting rs4668142 by single base extension; Primer 33 further comprises a primer for detecting rs2380261 by single base extension; Primer 34 further comprises a primer for detecting rs39059 by single base extension; Primer 35 further comprises a primer for detecting rs17756941 by single base extension; Primer 36 further comprises a primer for detecting rs245955 by single base extension; Primer 37 further comprises a primer for detecting rs245962 by single base extension; Primer 38 further comprises a primer for detecting rs266729 by single base extension; Primer 39 further comprises a primer for detecting rs3811951 by single base extension; Primer 40 further comprises a primer for detecting rs156019 by single base extension; Primer 41 further comprises a primer for detecting rs6234 by single base extension; Primer 42 further comprises a primer for detecting rs3856806 by single base extension; Primer 43 further comprises a primer for detecting rs10494366 by single base extension; Primer 44 further comprises a primer for detecting rs11977021 by single base extension; Primer 45 further comprises a primer for detecting rs2230806 by single base extension; Primer 46 further comprises a primer for detecting rs11212617 by single base extension; and/or Primer 47 further comprises a primer for detecting rs622342 by single base extension,

optionally wherein the primer for detecting rs10229583 by single base extension comprises the single-chain DNA sequence set forth in SEQ ID NO: 95; the primer for detecting rs10811661 by single base extension comprises the single-chain DNA sequence set forth in SEQ ID NO: 96; the primer for detecting rs10886471 by single base extension comprises the single-chain DNA sequence set forth in SEQ ID NO: 97; the primer for detecting rs1111875 by single base extension comprises the single-chain DNA sequence set forth in SEQ ID NO: 98; the primer for detecting rs12742393 by single base extension comprises the single-chain DNA sequence set forth in SEQ ID NO: 99; the primer for detecting rs12779790 by single base extension comprises the single-chain DNA sequence set forth in SEQ ID NO: 100; the primer for detecting rs13266634 by single base extension comprises the single-chain DNA sequence set forth in SEQ ID NO: 101; the primer for detecting rs1535500 by single base extension comprises the single-chain DNA sequence set forth in SEQ ID NO: 102; the primer for detecting rs16856187 by single base extension comprises the single-chain DNA sequence set forth in SEQ ID NO: 103; the primer for detecting rs16861329 by single base extension comprises the single-chain DNA sequence set forth in SEQ ID NO: 104; the primer for detecting rs1801282 by single base extension comprises the single-chain DNA sequence set forth in SEQ ID NO: 105; the primer for detecting rs2237892 by single base extension comprises the single-chain DNA sequence set forth in SEQ ID NO: 106; the primer for detecting rs340874 by single base extension comprises the single-chain DNA sequence set forth in SEQ ID NO: 107; the primer for detecting rs3786897 by single base extension comprises the single-chain DNA sequence set forth in SEQ ID NO: 108; the primer for detecting rs4430796 by single base extension comprises the single-chain DNA sequence set forth in SEQ ID NO: 109; the primer for detecting rs5219 by single base extension comprises the single-chain DNA sequence set forth in SEQ ID NO: 110; the primer for detecting rs6017317 by single base extension comprises the single-chain DNA sequence set forth in SEQ ID NO: 111; the primer for detecting rs6467136 by single base extension comprises the single-chain DNA sequence set forth in SEQ ID NO: 112; the primer for detecting rs6815464 by single base extension comprises the single-chain DNA sequence set forth in SEQ ID NO: 113; the primer for detecting rs7041847 by single base extension comprises the single-chain DNA sequence set forth in SEQ ID NO: 114; the primer for detecting rs7172432 by single base extension comprises the single-chain DNA sequence set forth in SEQ ID NO: 115; the primer for detecting rs7612463 by single base extension comprises the single-chain DNA sequence set forth in SEQ ID NO: 116; the primer for detecting rs7651090 by single base extension comprises the single-chain DNA sequence set forth in SEQ ID NO: 117; the primer for detecting rs7756992 by single base extension comprises the single-chain DNA sequence set forth in SEQ ID NO: 118; the primer for detecting rs780094 by single base extension comprises the single-chain DNA sequence set forth in SEQ ID NO: 119; the primer for detecting rs7903146 by single base extension comprises the single-chain DNA sequence set forth in SEQ ID NO: 120; the primer for detecting rs8050136 by single base extension comprises the single-chain DNA sequence set forth in SEQ ID NO: 121; the primer for detecting rs831571 by single base extension comprises the single-chain DNA sequence set forth in SEQ ID NO: 122; the primer for detecting rs9470794 by single base extension comprises the single-chain DNA sequence set forth in SEQ ID NO: 123; the primer for detecting rs2268388 by single base extension comprises the single-chain DNA sequence set forth in SEQ ID NO: 124; the primer for detecting rs9565164 by single base extension comprises the single-chain DNA sequence set forth in SEQ ID NO: 125; the primer for detecting rs4668142 by single base extension comprises the single-chain DNA sequence set forth in SEQ ID NO: 126; the primer for detecting rs2380261 by single base extension comprises the single-chain DNA sequence set forth in SEQ ID NO: 127; the primer for detecting rs39059 by single base extension comprises the single-chain DNA sequence set forth in SEQ ID NO: 128; the primer for detecting rs17756941 by single base extension comprises the single-chain DNA sequence set forth in SEQ ID NO: 129; the primer for detecting rs245955 by single base extension comprises the single-chain DNA sequence set forth in SEQ ID NO: 130; the primer for detecting rs245962 by single base extension comprises the single-chain DNA sequence set forth in SEQ ID NO: 131; the primer for detecting rs266729 by single base extension comprises the single-chain DNA sequence set forth in SEQ ID NO: 132; the primer for detecting rs3811951 by single base extension comprises the single-chain DNA sequence set forth in SEQ ID NO: 133; the primer for detecting rs156019 by single base extension comprises the single-chain DNA sequence set forth in SEQ ID NO: 134, the primer for detecting rs6234 by single base extension comprises the single-chain DNA sequence set forth in SEQ ID NO: 135; the primer for detecting rs3856806 by single base extension comprises the single-chain DNA sequence set forth in SEQ ID NO: 136; the primer for detecting rs10494366 by single base extension comprises the single-chain DNA sequence set forth in SEQ ID NO: 137; the primer for detecting rs11977021 by single base extension comprises the single-chain DNA sequence set forth in SEQ ID NO: 138; the primer for detecting rs2230806 by single base extension comprises the single-chain DNA sequence set forth in SEQ ID NO: 139; the primer for detecting rs11212617 by single base extension comprises the single-chain DNA sequence set forth in SEQ ID NO: 140; and/or the primer for detecting rs622342 by single base extension comprises the single-chain DNA sequence set forth in SEQ ID NO: 141.

### Embodiment 3

A kit for detecting an SNP (Single Nucleotide Polymorphism) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, wherein the kit comprises the primer set of Embodiment 1 or Embodiment 2.

### Embodiment 4

Use of the primer set of Embodiment 1 or Embodiment 2 or the kit of Embodiment 3 for detecting an SNP (Single Nucleotide Polymorphism) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs111212617, and rs622342.

### Embodiment 5

Use of the primer set of Embodiment 1 or Embodiment 2 or the kit of Embodiment 3 in the manufacture of a product for detecting an SNP (Single Nucleotide Polymorphism) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342.

Additional non-limiting embodiments are provided below:

### Embodiment 1

An isolated polynucleotide comprising a nucleic acid sequence having at least about 85%, at least about 90%, at least about 95%, at least about 99%, or 100% sequence homology or identity with any of SEQ ID NOs: 1-141.

### Embodiment 2

A set of isolated polynucleotides comprising nucleic acid sequences having at least about 85%, at least about 90%, at least about 95%, at least about 99%, or 100% sequence homology or identity with one or more of SEQ ID NOs: 1-141.

### Embodiment 3

The set of isolated polynucleotides of embodiment 2, which comprises two or more of the nucleic acid sequences set forth in SEQ ID NOs: 1-94.

### Embodiment 4

The set of isolated polynucleotides of embodiment 2, which comprises one or more of the nucleic acid sequences set forth in SEQ ID NOs: 95-141.

### Embodiment 5

The isolated polynucleotide of embodiment 1 or set of isolated polynucleotides of any of embodiments 2-4, which is for detecting one or more of the single nucleotide polymorphisms (SNPs) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342; and/or complementary sequences thereof; and/or sequences in linkage disequilibrium therewith.

### Embodiment 6

The set of isolated polynucleotides of embodiment 5, which comprises at least two amplification primers and at least one primer for single base extension for detecting the one or more SNPs.

### Embodiment 7

The set of isolated polynucleotides of embodiment 6, wherein:

the two amplification primers comprise nucleic acid sequences set forth in SEQ ID NOs: 1 and 2, respectively, and/or the primer for single base extension comprises the nucleic acid sequence set forth in SEQ ID NO: 95; and/or

the two amplification primers comprise nucleic acid sequences set forth in SEQ ID NOs: 3 and 4, respectively, and/or the primer for single base extension comprises the nucleic acid sequence set forth in SEQ ID NO: 96; and/or

the two amplification primers comprise nucleic acid sequences set forth in SEQ ID NOs: 5 and 6, respectively, and/or the primer for single base extension comprises the nucleic acid sequence set forth in SEQ ID NO: 97; and/or

the two amplification primers comprise nucleic acid sequences set forth in SEQ ID NOs: 7 and 8, respectively, and/or the primer for single base extension comprises the nucleic acid sequence set forth in SEQ ID NO: 98; and/or

the two amplification primers comprise nucleic acid sequences set forth in SEQ ID NOs: 9 and 10, respectively, and/or the primer for single base extension comprises the nucleic acid sequence set forth in SEQ ID NO: 99; and/or

the two amplification primers comprise nucleic acid sequences set forth in SEQ ID NOs: 11 and 12, respectively, and/or the primer for single base extension comprises the nucleic acid sequence set forth in SEQ ID NO: 100; and/or

the two amplification primers comprise nucleic acid sequences set forth in SEQ ID NOs: 13 and 14, respectively, and/or the primer for single base extension comprises the nucleic acid sequence set forth in SEQ ID NO: 101; and/or

the two amplification primers comprise nucleic acid sequences set forth in SEQ ID NOs: 15 and 16, respectively, and/or the primer for single base extension comprises the nucleic acid sequence set forth in SEQ ID NO: 102; and/or

the two amplification primers comprise nucleic acid sequences set forth in SEQ ID NOs: 17 and 18, respectively, and/or the primer for single base extension comprises the nucleic acid sequence set forth in SEQ ID NO: 103; and/or

the two amplification primers comprise nucleic acid sequences set forth in SEQ ID NOs: 19 and 20, respectively, and/or the primer for single base extension comprises the nucleic acid sequence set forth in SEQ ID NO: 104; and/or

the two amplification primers comprise nucleic acid sequences set forth in SEQ ID NOs: 21 and 22, respectively, and/or the primer for single base extension comprises the nucleic acid sequence set forth in SEQ ID NO: 105; and/or

the two amplification primers comprise nucleic acid sequences set forth in SEQ ID NOs: 23 and 24, respectively, and/or the primer for single base extension comprises the nucleic acid sequence set forth in SEQ ID NO: 106; and/or

the two amplification primers comprise nucleic acid sequences set forth in SEQ ID NOs: 25 and 26, respectively, and/or the primer for single base extension comprises the nucleic acid sequence set forth in SEQ ID NO: 107; and/or

the two amplification primers comprise nucleic acid sequences set forth in SEQ ID NOs: 27 and 28, respectively, and/or the primer for single base extension comprises the nucleic acid sequence set forth in SEQ ID NO: 108; and/or

the two amplification primers comprise nucleic acid sequences set forth in SEQ ID NOs: 29 and 30, respectively, and/or the primer for single base extension comprises the nucleic acid sequence set forth in SEQ ID NO: 109; and/or

the two amplification primers comprise nucleic acid sequences set forth in SEQ ID NOs: 31 and 32, respectively, and/or the primer for single base extension comprises the nucleic acid sequence set forth in SEQ ID NO: 110; and/or

the two amplification primers comprise nucleic acid sequences set forth in SEQ ID NOs: 33 and 34, respectively, and/or the primer for single base extension comprises the nucleic acid sequence set forth in SEQ ID NO: 111; and/or

the two amplification primers comprise nucleic acid sequences set forth in SEQ ID NOs: 35 and 36, respectively, and/or the primer for single base extension comprises the nucleic acid sequence set forth in SEQ ID NO: 112; and/or

the two amplification primers comprise nucleic acid sequences set forth in SEQ ID NOs: 37 and 38, respectively, and/or the primer for single base extension comprises the nucleic acid sequence set forth in SEQ ID NO: 113; and/or

the two amplification primers comprise nucleic acid sequences set forth in SEQ ID NOs: 39 and 40, respectively, and/or the primer for single base extension comprises the nucleic acid sequence set forth in SEQ ID NO: 114; and/or

the two amplification primers comprise nucleic acid sequences set forth in SEQ ID NOs: 41 and 42, respectively, and/or the primer for single base extension comprises the nucleic acid sequence set forth in SEQ ID NO: 115; and/or

the two amplification primers comprise nucleic acid sequences set forth in SEQ ID NOs: 43 and 44, respectively, and/or the primer for single base extension comprises the nucleic acid sequence set forth in SEQ ID NO: 116; and/or

the two amplification primers comprise nucleic acid sequences set forth in SEQ ID NOs: 45 and 46, respectively, and/or the primer for single base extension comprises the nucleic acid sequence set forth in SEQ ID NO: 117; and/or

the two amplification primers comprise nucleic acid sequences set forth in SEQ ID NOs: 47 and 48, respectively, and/or the primer for single base extension comprises the nucleic acid sequence set forth in SEQ ID NO: 118; and/or

the two amplification primers comprise nucleic acid sequences set forth in SEQ ID NOs: 49 and 50, respectively, and/or the primer for single base extension comprises the nucleic acid sequence set forth in SEQ ID NO: 119; and/or

the two amplification primers comprise nucleic acid sequences set forth in SEQ ID NOs: 51 and 52, respectively, and/or the primer for single base extension comprises the nucleic acid sequence set forth in SEQ ID NO: 120; and/or

the two amplification primers comprise nucleic acid sequences set forth in SEQ ID NOs: 53 and 54, respectively, and/or the primer for single base extension comprises the nucleic acid sequence set forth in SEQ ID NO: 121; and/or

the two amplification primers comprise nucleic acid sequences set forth in SEQ ID NOs: 55 and 56, respectively, and/or the primer for single base extension comprises the nucleic acid sequence set forth in SEQ ID NO: 122; and/or

the two amplification primers comprise nucleic acid sequences set forth in SEQ ID NOs: 57 and 58, respectively, and/or the primer for single base extension comprises the nucleic acid sequence set forth in SEQ ID NO: 123; and/or

the two amplification primers comprise nucleic acid sequences set forth in SEQ ID NOs: 59 and 60, respectively, and/or the primer for single base extension comprises the nucleic acid sequence set forth in SEQ ID NO: 124; and/or

the two amplification primers comprise nucleic acid sequences set forth in SEQ ID NOs: 61 and 62, respectively, and/or the primer for single base extension comprises the nucleic acid sequence set forth in SEQ ID NO: 125; and/or

the two amplification primers comprise nucleic acid sequences set forth in SEQ ID NOs: 63 and 64, respectively, and/or the primer for single base extension comprises the nucleic acid sequence set forth in SEQ ID NO: 126; and/or

the two amplification primers comprise nucleic acid sequences set forth in SEQ ID NOs: 65 and 66, respectively, and/or the primer for single base extension comprises the nucleic acid sequence set forth in SEQ ID NO: 127; and/or

the two amplification primers comprise nucleic acid sequences set forth in SEQ ID NOs: 67 and 68, respectively, and/or the primer for single base extension comprises the nucleic acid sequence set forth in SEQ ID NO: 128; and/or

the two amplification primers comprise nucleic acid sequences set forth in SEQ ID NOs: 69 and 70, respectively, and/or the primer for single base extension comprises the nucleic acid sequence set forth in SEQ ID NO: 129; and/or

the two amplification primers comprise nucleic acid sequences set forth in SEQ ID NOs: 71 and 72, respectively, and/or the primer for single base extension comprises the nucleic acid sequence set forth in SEQ ID NO: 130; and/or

the two amplification primers comprise nucleic acid sequences set forth in SEQ ID NOs: 73 and 74, respectively, and/or the primer for single base extension comprises the nucleic acid sequence set forth in SEQ ID NO: 131; and/or

the two amplification primers comprise nucleic acid sequences set forth in SEQ ID NOs: 75 and 76, respectively, and/or the primer for single base extension comprises the nucleic acid sequence set forth in SEQ ID NO: 132; and/or

the two amplification primers comprise nucleic acid sequences set forth in SEQ ID NOs: 77 and 78, respectively, and/or the primer for single base extension comprises the nucleic acid sequence set forth in SEQ ID NO: 133; and/or

the two amplification primers comprise nucleic acid sequences set forth in SEQ ID NOs: 79 and 80, respectively, and/or the primer for single base extension comprises the nucleic acid sequence set forth in SEQ ID NO: 134; and/or

the two amplification primers comprise nucleic acid sequences set forth in SEQ ID NOs: 81 and 82, respectively, and/or the primer for single base extension comprises the nucleic acid sequence set forth in SEQ ID NO: 135; and/or

the two amplification primers comprise nucleic acid sequences set forth in SEQ ID NOs: 83 and 84, respectively, and/or the primer for single base extension comprises the nucleic acid sequence set forth in SEQ ID NO: 136; and/or

the two amplification primers comprise nucleic acid sequences set forth in SEQ ID NOs: 85 and 86, respectively, and/or the primer for single base extension comprises the nucleic acid sequence set forth in SEQ ID NO: 137; and/or

the two amplification primers comprise nucleic acid sequences set forth in SEQ ID NOs: 87 and 88, respectively, and/or the primer for single base extension comprises the nucleic acid sequence set forth in SEQ ID NO: 138; and/or

the two amplification primers comprise nucleic acid sequences set forth in SEQ ID NOs: 89 and 90, respectively, and/or the primer for single base extension comprises the nucleic acid sequence set forth in SEQ ID NO: 139; and/or

the two amplification primers comprise nucleic acid sequences set forth in SEQ ID NOs: 91 and 92, respectively, and/or the primer for single base extension comprises the nucleic acid sequence set forth in SEQ ID NO: 140; and/or

the two amplification primers comprise nucleic acid sequences set forth in SEQ ID NOs: 93 and 94, respectively, and/or the primer for single base extension comprises the nucleic acid sequence set forth in SEQ ID NO: 141.

### Embodiment 8

The isolated polynucleotide or the set of isolated polynucleotides of any of the preceding embodiments, which is for detection of one or more SNPs associated with diabetes mellitus and/or a disease or condition related to diabetes mellitus.

### Embodiment 9

The isolated polynucleotide or the set of isolated polynucleotides of embodiment 8, which is for detection of one or more SNPs associated with type 2 diabetes mellitus, diabetic nephropathy, diabetic retinitis, diabetic cardiomyopathy (e.g., elderly diabetic cardiomyopathy), and/or drug resistance to an anti-diabetes medication.

### Embodiment 10

The isolated polynucleotide or the set of isolated polynucleotides of embodiment 9, wherein the SNPs associated with type 2 diabetes mellitus comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329.

### Embodiment 11

The isolated polynucleotide or the set of isolated polynucleotides of embodiment 9 or 10, wherein the SNPs associated with diabetic nephropathy comprise any one or more of rs2268388 and rs1801282.

### Embodiment 12

The isolated polynucleotide or the set of isolated polynucleotides of any of embodiments 9-11, wherein the SNPs associated with diabetic retinitis comprise any one or more of rs9565164, rs4668142, rs2380261, rs39059, rs17756941, rs245955, and rs245962.

### Embodiment 13

The isolated polynucleotide or the set of isolated polynucleotides of any of embodiments 9-12, wherein the SNPs associated with diabetic cardiomyopathy comprise any one or more of rs266729, rs3811951, rs156019, rs6234, rs1801282, and rs3856806.

### Embodiment 14

The isolated polynucleotide or the set of isolated polynucleotides of any of embodiments 9-13, wherein the SNPs associated with the drug resistance comprise any one or more of rs13266634, rs10494366, rs2237892, rs11977021, rs7651090, rs5219, rs7903146, rs1801282, rs6467136, rs2230806, rs11212617, and rs622342.

### Embodiment 15

The isolated polynucleotide or the set of isolated polynucleotides of any of embodiments 9-14, wherein the anti-diabetes medication comprises any one or more of Repaglinide, Rosiglitazone, Metformin, Gliclazide, and Pioglitazone.

### Embodiment 16

The isolated polynucleotide or the set of isolated polynucleotides of embodiment 15, wherein the SNPs associated with Repaglinide resistance comprise any one or more of rs13266634, rs10494366, rs2237892, rs11977021, rs7651090, rs5219, and rs7903146.

### Embodiment 17

The isolated polynucleotide or the set of isolated polynucleotides of embodiment 15 or 16, wherein the SNPs associated with Rosiglitazone resistance comprise any one or more of rs13266634, rs2237892, rs1801282, rs6467136, and rs2230806.

### Embodiment 18

The isolated polynucleotide or the set of isolated polynucleotides of any of embodiments 15-17, wherein the SNPs associated with Metformin resistance comprise any one or more of rs11212617 and rs622342.

### Embodiment 19

The isolated polynucleotide or the set of isolated polynucleotides of any of embodiments 15-18, wherein the SNPs associated with Gliclazide resistance comprise rs5219.

### Embodiment 20

The isolated polynucleotide or the set of isolated polynucleotides of any of embodiments 15-19, wherein the SNPs associated with Pioglitazone resistance comprise rs1801282.

### Embodiment 21

The isolated polynucleotide or the set of isolated polynucleotides of any of embodiments 8-20, wherein the diabetes mellitus and/or disease or condition related to diabetes mellitus are in an East Asian population.

### Embodiment 22

A kit comprising the isolated polynucleotide or the set of isolated polynucleotides of any of the preceding embodiments.

### Embodiment 23

The kit of embodiment 22, further comprising instructions for using the isolated polynucleotide or the set of isolated polynucleotides to conduct a companion diagnostic test.

### Embodiment 24

The kit of embodiment 23, wherein the companion diagnostic test is for monitoring treatment of diabetes mellitus and/or a disease or condition related to diabetes mellitus.

### Embodiment 25

Use of the isolated polynucleotide, the set of isolated polynucleotides, or the kit of any of the preceding embodiments, for detecting one or more of the single nucleotide polymorphisms (SNPs) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

### Embodiment 26

Use of the isolated polynucleotide, the set of isolated polynucleotides, or the kit of any of the preceding embodiments, for the manufacture of a product for detecting one or more of the single nucleotide polymorphisms (SNPs) selected from the group consisting of rs10229583, rs10811661, rs10886471, rs111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

### Embodiment 27

A method for risk assessment, diagnosis, prognosis and/or treatment monitoring of diabetes mellitus and/or a disease or condition related to diabetes mellitus in a subject, the method comprising:

detecting one or more single nucleotide polymorphisms (SNPs) in a biological sample from the subject, wherein the SNPs are selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

### Embodiment 28

The method of embodiment 27, wherein the one or more SNPs are detected using a primer for the SNP or SNPs.

### Embodiment 29

The method of embodiment 28, wherein the primer comprises the nucleic acid sequence set forth in any of SEQ ID NOs: 1-141.

### Embodiment 30

The method of embodiment 28 or 29, wherein a plurality of primers are used to detect the SNP or SNPs, and the plurality of primers comprise at least three of the nucleic acid sequences set forth in SEQ ID NOs: 1-141.

### Embodiment 31

The method of any one of embodiments 28-30, wherein the primer comprises two or more of the nucleic acid sequences set forth in SEQ ID NOs: 1-94.

### Embodiment 32

The method of any one of embodiments 28-30, wherein the primer comprises one or more of the nucleic acid sequences set forth in SEQ ID NOs: 95-141.

### Embodiment 33

The method of any one of embodiments 27-32, wherein the SNP or SNPs are detected using at least two amplification primers.

### Embodiment 34

The method of any one of embodiments 27-33, wherein the SNP or SNPs are detected using at least one primer for single base extension.

### Embodiment 35

The method of any one of embodiments 27-34, wherein the SNP or SNPs are detected using:

two amplification primers comprising nucleic acid sequences set forth in SEQ ID NOs: 1 and 2, respectively, and/or a primer for single base extension comprising the nucleic acid sequence set forth in SEQ ID NO: 95; and/or

two amplification primers comprising nucleic acid sequences set forth in SEQ ID NOs: 3 and 4, respectively, and/or a primer for single base extension comprising the nucleic acid sequence set forth in SEQ ID NO: 96; and/or

two amplification primers comprising nucleic acid sequences set forth in SEQ ID NOs: 5 and 6, respectively, and/or a primer for single base extension comprising the nucleic acid sequence set forth in SEQ ID NO: 97; and/or

two amplification primers comprising nucleic acid sequences set forth in SEQ ID NOs: 7 and 8, respectively, and/or a primer for single base extension comprising the nucleic acid sequence set forth in SEQ ID NO: 98; and/or

two amplification primers comprising nucleic acid sequences set forth in SEQ ID NOs: 9 and 10, respectively, and/or a primer for single base extension comprising the nucleic acid sequence set forth in SEQ ID NO: 99; and/or

two amplification primers comprising nucleic acid sequences set forth in SEQ ID NOs: 11 and 12, respectively, and/or a primer for single base extension comprising the nucleic acid sequence set forth in SEQ ID NO: 100; and/or

two amplification primers comprising nucleic acid sequences set forth in SEQ ID NOs: 13 and 14, respectively, and/or a primer for single base extension comprising the nucleic acid sequence set forth in SEQ ID NO: 101; and/or

two amplification primers comprising nucleic acid sequences set forth in SEQ ID NOs: 15 and 16, respectively, and/or a primer for single base extension comprising the nucleic acid sequence set forth in SEQ ID NO: 102; and/or

two amplification primers comprising nucleic acid sequences set forth in SEQ ID NOs: 17 and 18, respectively, and/or a primer for single base extension comprising the nucleic acid sequence set forth in SEQ ID NO: 103; and/or

two amplification primers comprising nucleic acid sequences set forth in SEQ ID NOs: 19 and 20, respectively, and/or a primer for single base extension comprising the nucleic acid sequence set forth in SEQ ID NO: 104; and/or

two amplification primers comprising nucleic acid sequences set forth in SEQ ID NOs: 21 and 22, respectively, and/or a primer for single base extension comprising the nucleic acid sequence set forth in SEQ ID NO: 105; and/or

two amplification primers comprising nucleic acid sequences set forth in SEQ ID NOs: 23 and 24, respectively, and/or a primer for single base extension comprising the nucleic acid sequence set forth in SEQ ID NO: 106; and/or

two amplification primers comprising nucleic acid sequences set forth in SEQ ID NOs: 25 and 26, respectively, and/or a primer for single base extension comprising the nucleic acid sequence set forth in SEQ ID NO: 107; and/or

two amplification primers comprising nucleic acid sequences set forth in SEQ ID NOs: 27 and 28, respectively, and/or a primer for single base extension comprising the nucleic acid sequence set forth in SEQ ID NO: 108; and/or

two amplification primers comprising nucleic acid sequences set forth in SEQ ID NOs: 29 and 30, respectively, and/or a primer for single base extension comprising the nucleic acid sequence set forth in SEQ ID NO: 109; and/or

two amplification primers comprising nucleic acid sequences set forth in SEQ ID NOs: 31 and 32, respectively, and/or a primer for single base extension comprising the nucleic acid sequence set forth in SEQ ID NO: 110; and/or

two amplification primers comprising nucleic acid sequences set forth in SEQ ID NOs: 33 and 34, respectively, and/or a primer for single base extension comprising the nucleic acid sequence set forth in SEQ ID NO: 111; and/or

two amplification primers comprising nucleic acid sequences set forth in SEQ ID NOs: 35 and 36, respectively, and/or a primer for single base extension comprising the nucleic acid sequence set forth in SEQ ID NO: 112; and/or

two amplification primers comprising nucleic acid sequences set forth in SEQ ID NOs: 37 and 38, respectively, and/or a primer for single base extension comprising the nucleic acid sequence set forth in SEQ ID NO: 113; and/or

two amplification primers comprising nucleic acid sequences set forth in SEQ ID NOs: 39 and 40, respectively, and/or a primer for single base extension comprising the nucleic acid sequence set forth in SEQ ID NO: 114; and/or

two amplification primers comprising nucleic acid sequences set forth in SEQ ID NOs: 41 and 42, respectively, and/or a primer for single base extension comprising the nucleic acid sequence set forth in SEQ ID NO: 115; and/or

two amplification primers comprising nucleic acid sequences set forth in SEQ ID NOs: 43 and 44, respectively, and/or a primer for single base extension comprising the nucleic acid sequence set forth in SEQ ID NO: 116; and/or

two amplification primers comprising nucleic acid sequences set forth in SEQ ID NOs: 45 and 46, respectively, and/or a primer for single base extension comprising the nucleic acid sequence set forth in SEQ ID NO: 117; and/or

two amplification primers comprising nucleic acid sequences set forth in SEQ ID NOs: 47 and 48, respectively, and/or a primer for single base extension comprising the nucleic acid sequence set forth in SEQ ID NO: 118; and/or

two amplification primers comprising nucleic acid sequences set forth in SEQ ID NOs: 49 and 50, respectively, and/or a primer for single base extension comprising the nucleic acid sequence set forth in SEQ ID NO: 119; and/or

two amplification primers comprising nucleic acid sequences set forth in SEQ ID NOs: 51 and 52, respectively, and/or a primer for single base extension comprising the nucleic acid sequence set forth in SEQ ID NO: 120; and/or

two amplification primers comprising nucleic acid sequences set forth in SEQ ID NOs: 53 and 54, respectively, and/or a primer for single base extension comprising the nucleic acid sequence set forth in SEQ ID NO: 121; and/or

two amplification primers comprising nucleic acid sequences set forth in SEQ ID NOs: 55 and 56, respectively, and/or a primer for single base extension comprising the nucleic acid sequence set forth in SEQ ID NO: 122; and/or

two amplification primers comprising nucleic acid sequences set forth in SEQ ID NOs: 57 and 58, respectively, and/or a primer for single base extension comprising the nucleic acid sequence set forth in SEQ ID NO: 123; and/or

two amplification primers comprising nucleic acid sequences set forth in SEQ ID NOs: 59 and 60, respectively, and/or a primer for single base extension comprising the nucleic acid sequence set forth in SEQ ID NO: 124; and/or

two amplification primers comprising nucleic acid sequences set forth in SEQ ID NOs: 61 and 62, respectively, and/or a primer for single base extension comprising the nucleic acid sequence set forth in SEQ ID NO: 125; and/or

two amplification primers comprising nucleic acid sequences set forth in SEQ ID NOs: 63 and 64, respectively, and/or a primer for single base extension comprising the nucleic acid sequence set forth in SEQ ID NO: 126; and/or

two amplification primers comprising nucleic acid sequences set forth in SEQ ID NOs: 65 and 66, respectively, and/or a primer for single base extension comprising the nucleic acid sequence set forth in SEQ ID NO: 127; and/or

two amplification primers comprising nucleic acid sequences set forth in SEQ ID NOs: 67 and 68, respectively, and/or a primer for single base extension comprising the nucleic acid sequence set forth in SEQ ID NO: 128; and/or

two amplification primers comprising nucleic acid sequences set forth in SEQ ID NOs: 69 and 70, respectively, and/or a primer for single base extension comprising the nucleic acid sequence set forth in SEQ ID NO: 129; and/or

two amplification primers comprising nucleic acid sequences set forth in SEQ ID NOs: 71 and 72, respectively, and/or a primer for single base extension comprising the nucleic acid sequence set forth in SEQ ID NO: 130; and/or

two amplification primers comprising nucleic acid sequences set forth in SEQ ID NOs: 73 and 74, respectively, and/or a primer for single base extension comprising the nucleic acid sequence set forth in SEQ ID NO: 131; and/or

two amplification primers comprising nucleic acid sequences set forth in SEQ ID NOs: 75 and 76, respectively, and/or a primer for single base extension comprising the nucleic acid sequence set forth in SEQ ID NO: 132; and/or

two amplification primers comprising nucleic acid sequences set forth in SEQ ID NOs: 77 and 78, respectively, and/or a primer for single base extension comprising the nucleic acid sequence set forth in SEQ ID NO: 133; and/or

two amplification primers comprising nucleic acid sequences set forth in SEQ ID NOs: 79 and 80, respectively, and/or a primer for single base extension comprising the nucleic acid sequence set forth in SEQ ID NO: 134; and/or

two amplification primers comprising nucleic acid sequences set forth in SEQ ID NOs: 81 and 82, respectively, and/or a primer for single base extension comprising the nucleic acid sequence set forth in SEQ ID NO: 135; and/or

two amplification primers comprising nucleic acid sequences set forth in SEQ ID NOs: 83 and 84, respectively, and/or a primer for single base extension comprising the nucleic acid sequence set forth in SEQ ID NO: 136; and/or

two amplification primers comprising nucleic acid sequences set forth in SEQ ID NOs: 85 and 86, respectively, and/or a primer for single base extension comprising the nucleic acid sequence set forth in SEQ ID NO: 137; and/or

two amplification primers comprising nucleic acid sequences set forth in SEQ ID NOs: 87 and 88, respectively, and/or a primer for single base extension comprising the nucleic acid sequence set forth in SEQ ID NO: 138; and/or

two amplification primers comprising nucleic acid sequences set forth in SEQ ID NOs: 89 and 90, respectively, and/or a primer for single base extension comprising the nucleic acid sequence set forth in SEQ ID NO: 139; and/or

two amplification primers comprising nucleic acid sequences set forth in SEQ ID NOs: 91 and 92, respectively, and/or a primer for single base extension comprising the nucleic acid sequence set forth in SEQ ID NO: 140; and/or

two amplification primers comprising nucleic acid sequences set forth in SEQ ID NOs: 93 and 94, respectively, and/or a primer for single base extension comprising the nucleic acid sequence set forth in SEQ ID NO: 141.

### Embodiment 36

The method of any one of embodiments 27-35, wherein the one or more SNPs are associated with type 2 diabetes mellitus, diabetic nephropathy, diabetic retinitis, diabetic cardiomyopathy (e.g., elderly diabetic cardiomyopathy), and/or drug resistance to an anti-diabetes medication.

### Embodiment 37

The method of any one of embodiments 27-36, wherein the diabetes mellitus and/or disease or condition related to diabetes mellitus comprises type 2 diabetes mellitus, diabetic nephropathy, diabetic retinitis, diabetic cardiomyopathy (e.g., elderly diabetic cardiomyopathy), and/or drug resistance to an anti-diabetes medication.

### Embodiment 38

The method of embodiment 36 or 37, wherein the SNPs associated with type 2 diabetes mellitus comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329.

### Embodiment 39

The method of embodiment 36 or 37, wherein the SNPs associated with diabetic nephropathy comprise any one or more of rs2268388 and rs1801282.

### Embodiment 40

The method of embodiment 36 or 37, wherein the SNPs associated with diabetic retinitis comprise any one or more of rs9565164, rs4668142, rs2380261, rs39059, rs17756941, rs245955, and rs245962.

### Embodiment 41

The method of embodiment 36 or 37, wherein the SNPs associated with diabetic cardiomyopathy comprise any one or more of rs266729, rs3811951, rs156019, rs6234, rs1801282, and rs3856806.

### Embodiment 42

The method of embodiment 36 or 37, wherein the SNPs associated with the drug resistance comprise any one or more of rs13266634, rs10494366, rs2237892, rs11977021, rs7651090, rs5219, rs7903146, rs1801282, rs6467136, rs2230806, rs11212617, and rs622342.

### Embodiment 43

The method of embodiment 42, wherein the anti-diabetes medication comprises any one or more of Repaglinide, Rosiglitazone, Metformin, Gliclazide, and Pioglitazone.

### Embodiment 44

The method of embodiment 43, wherein the SNPs associated with Repaglinide resistance comprise any one or more of rs13266634, rs10494366, rs2237892, rs11977021, rs7651090, rs5219, and rs7903146.

### Embodiment 45

The method of embodiment 43 or 44, wherein the SNPs associated with Rosiglitazone resistance comprise any one or more of rs13266634, rs2237892, rs1801282, rs6467136, and rs2230806.

### Embodiment 46

The method of any one of embodiments 43-45, wherein the SNPs associated with Metformin resistance comprise any one or more of rs11212617 and rs622342.

### Embodiment 47

The method of any one of embodiments 43-46, wherein the SNPs associated with Gliclazide resistance comprise rs5219.

### Embodiment 48

The method of any one of embodiments 43-47, wherein the SNPs associated with Pioglitazone resistance comprise rs1801282.

### Embodiment 49

The method of any one of embodiments 27-48, wherein the diabetes mellitus and/or disease or condition related to diabetes mellitus are in an East Asian population.

### Embodiment 50

The method of any one of embodiments 27-49, which further comprises treating diabetes mellitus and/or a disease or condition related to diabetes mellitus in the subject.

### Embodiment 51

The method of embodiment 50, wherein the treatment of diabetes mellitus and/or a disease or condition related to diabetes mellitus in the subject is adjusted based on the SNP(s) detection result in the biological sample from the subject.

1. An isolated polynucleotide comprising a nucleic acid sequence having at least about 85%, at least about 90%, at least about 95%, at least about 99%, or 100% sequence homology or identity with any of SEQ ID NOs: 1-141.

2. A set of isolated polynucleotides comprising nucleic acid sequences having at least about 85%, at least about 90%, at least about 95%, at least about 99%, or 100% sequence homology or identity with one or more of SEQ ID NOs: 1-141.

3. The set of isolated polynucleotides of claim 2, which comprises two or more of the nucleic acid sequences set forth in SEQ ID NOs: 1-94.

4. The set of isolated polynucleotides of claim 2, which comprises one or more of the nucleic acid sequences set forth in SEQ ID NOs: 95-141.

5. The isolated polynucleotide of claim 1 or set of isolated polynucleotides of any of claims 2-4, which is for detecting one or more of the single nucleotide polymorphisms (SNPs) selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342; and/or complementary sequences thereof; and/or sequences in linkage disequilibrium therewith.

6. The set of isolated polynucleotides of claim 5, which comprises at least two amplification primers and at least one primer for single base extension for detecting the one or more SNPs.

7. The set of isolated polynucleotides of claim 6, wherein:
the two amplification primers comprise nucleic acid sequences set forth in SEQ ID NOs: 1 and 2, respectively, and/or the primer for single base extension comprises the nucleic acid sequence set forth in SEQ ID NO: 95; and/or
the two amplification primers comprise nucleic acid sequences set forth in SEQ ID NOs: 3 and 4, respectively, and/or the primer for single base extension comprises the nucleic acid sequence set forth in SEQ ID NO: 96; and/or
the two amplification primers comprise nucleic acid sequences set forth in SEQ ID NOs: 5 and 6, respectively, and/or the primer for single base extension comprises the nucleic acid sequence set forth in SEQ ID NO: 97; and/or
the two amplification primers comprise nucleic acid sequences set forth in SEQ ID NOs: 7 and 8, respectively, and/or the primer for single base extension comprises the nucleic acid sequence set forth in SEQ ID NO: 98; and/or
the two amplification primers comprise nucleic acid sequences set forth in SEQ ID NOs: 9 and 10, respectively, and/or the primer for single base extension comprises the nucleic acid sequence set forth in SEQ ID NO: 99; and/or
the two amplification primers comprise nucleic acid sequences set forth in SEQ ID NOs: 11 and 12, respectively, and/or the primer for single base extension comprises the nucleic acid sequence set forth in SEQ ID NO: 100; and/or
the two amplification primers comprise nucleic acid sequences set forth in SEQ ID NOs: 13 and 14, respectively, and/or the primer for single base extension comprises the nucleic acid sequence set forth in SEQ ID NO: 101; and/or
the two amplification primers comprise nucleic acid sequences set forth in SEQ ID NOs: 15 and 16, respectively, and/or the primer for single base extension comprises the nucleic acid sequence set forth in SEQ ID NO: 102; and/or
the two amplification primers comprise nucleic acid sequences set forth in SEQ ID NOs: 17 and 18, respectively, and/or the primer for single base extension comprises the nucleic acid sequence set forth in SEQ ID NO: 103; and/or
the two amplification primers comprise nucleic acid sequences set forth in SEQ ID NOs: 19 and 20, respectively, and/or the primer for single base extension comprises the nucleic acid sequence set forth in SEQ ID NO: 104; and/or
the two amplification primers comprise nucleic acid sequences set forth in SEQ ID NOs: 21 and 22, respectively, and/or the primer for single base extension comprises the nucleic acid sequence set forth in SEQ ID NO: 105; and/or
the two amplification primers comprise nucleic acid sequences set forth in SEQ ID NOs: 23 and 24, respectively, and/or the primer for single base extension comprises the nucleic acid sequence set forth in SEQ ID NO: 106; and/or
the two amplification primers comprise nucleic acid sequences set forth in SEQ ID NOs: 25 and 26, respectively, and/or the primer for single base extension comprises the nucleic acid sequence set forth in SEQ ID NO: 107; and/or
the two amplification primers comprise nucleic acid sequences set forth in SEQ ID NOs: 27 and 28, respectively, and/or the primer for single base extension comprises the nucleic acid sequence set forth in SEQ ID NO: 108; and/or
the two amplification primers comprise nucleic acid sequences set forth in SEQ ID NOs: 29 and 30, respectively, and/or the primer for single base extension comprises the nucleic acid sequence set forth in SEQ ID NO: 109; and/or
the two amplification primers comprise nucleic acid sequences set forth in SEQ ID NOs: 31 and 32, respectively, and/or the primer for single base extension comprises the nucleic acid sequence set forth in SEQ ID NO: 110; and/or
the two amplification primers comprise nucleic acid sequences set forth in SEQ ID NOs: 33 and 34, respectively, and/or the primer for single base extension comprises the nucleic acid sequence set forth in SEQ ID NO: 111; and/or
the two amplification primers comprise nucleic acid sequences set forth in SEQ ID NOs: 35 and 36, respectively, and/or the primer for single base extension comprises the nucleic acid sequence set forth in SEQ ID NO: 112; and/or
the two amplification primers comprise nucleic acid sequences set forth in SEQ ID NOs: 37 and 38, respectively, and/or the primer for single base extension comprises the nucleic acid sequence set forth in SEQ ID NO: 113; and/or
the two amplification primers comprise nucleic acid sequences set forth in SEQ ID NOs: 39 and 40, respectively, and/or the primer for single base extension comprises the nucleic acid sequence set forth in SEQ ID NO: 114; and/or
the two amplification primers comprise nucleic acid sequences set forth in SEQ ID NOs: 41 and 42, respectively, and/or the primer for single base extension comprises the nucleic acid sequence set forth in SEQ ID NO: 115; and/or
the two amplification primers comprise nucleic acid sequences set forth in SEQ ID NOs: 43 and 44, respectively, and/or the primer for single base extension comprises the nucleic acid sequence set forth in SEQ ID NO: 116; and/or
the two amplification primers comprise nucleic acid sequences set forth in SEQ ID NOs: 45 and 46, respectively, and/or the primer for single base extension comprises the nucleic acid sequence set forth in SEQ ID NO: 117; and/or
the two amplification primers comprise nucleic acid sequences set forth in SEQ ID NOs: 47 and 48, respectively, and/or the primer for single base extension comprises the nucleic acid sequence set forth in SEQ ID NO: 118; and/or
the two amplification primers comprise nucleic acid sequences set forth in SEQ ID NOs: 49 and 50, respectively, and/or the primer for single base extension comprises the nucleic acid sequence set forth in SEQ ID NO: 119; and/or
the two amplification primers comprise nucleic acid sequences set forth in SEQ ID NOs: 51 and 52, respectively, and/or the primer for single base extension comprises the nucleic acid sequence set forth in SEQ ID NO: 120; and/or
the two amplification primers comprise nucleic acid sequences set forth in SEQ ID NOs: 53 and 54, respectively, and/or the primer for single base extension comprises the nucleic acid sequence set forth in SEQ ID NO: 121; and/or
the two amplification primers comprise nucleic acid sequences set forth in SEQ ID NOs: 55 and 56, respectively, and/or the primer for single base extension comprises the nucleic acid sequence set forth in SEQ ID NO: 122; and/or
the two amplification primers comprise nucleic acid sequences set forth in SEQ ID NOs: 57 and 58, respectively, and/or the primer for single base extension comprises the nucleic acid sequence set forth in SEQ ID NO: 123; and/or
the two amplification primers comprise nucleic acid sequences set forth in SEQ ID NOs: 59 and 60, respectively, and/or the primer for single base extension comprises the nucleic acid sequence set forth in SEQ ID NO: 124; and/or
the two amplification primers comprise nucleic acid sequences set forth in SEQ ID NOs: 61 and 62, respectively, and/or the primer for single base extension comprises the nucleic acid sequence set forth in SEQ ID NO: 125; and/or
the two amplification primers comprise nucleic acid sequences set forth in SEQ ID NOs: 63 and 64, respectively, and/or the primer for single base extension comprises the nucleic acid sequence set forth in SEQ ID NO: 126; and/or
the two amplification primers comprise nucleic acid sequences set forth in SEQ ID NOs: 65 and 66, respectively, and/or the primer for single base extension comprises the nucleic acid sequence set forth in SEQ ID NO: 127; and/or
the two amplification primers comprise nucleic acid sequences set forth in SEQ ID NOs: 67 and 68, respectively, and/or the primer for single base extension comprises the nucleic acid sequence set forth in SEQ ID NO: 128; and/or
the two amplification primers comprise nucleic acid sequences set forth in SEQ ID NOs: 69 and 70, respectively, and/or the primer for single base extension comprises the nucleic acid sequence set forth in SEQ ID NO: 129; and/or
the two amplification primers comprise nucleic acid sequences set forth in SEQ ID NOs: 71 and 72, respectively, and/or the primer for single base extension comprises the nucleic acid sequence set forth in SEQ ID NO: 130; and/or
the two amplification primers comprise nucleic acid sequences set forth in SEQ ID NOs: 73 and 74, respectively, and/or the primer for single base extension comprises the nucleic acid sequence set forth in SEQ ID NO: 131; and/or
the two amplification primers comprise nucleic acid sequences set forth in SEQ ID NOs: 75 and 76, respectively, and/or the primer for single base extension comprises the nucleic acid sequence set forth in SEQ ID NO: 132; and/or
the two amplification primers comprise nucleic acid sequences set forth in SEQ ID NOs: 77 and 78, respectively, and/or the primer for single base extension comprises the nucleic acid sequence set forth in SEQ ID NO: 133; and/or
the two amplification primers comprise nucleic acid sequences set forth in SEQ ID NOs: 79 and 80, respectively, and/or the primer for single base extension comprises the nucleic acid sequence set forth in SEQ ID NO: 134; and/or
the two amplification primers comprise nucleic acid sequences set forth in SEQ ID NOs: 81 and 82, respectively, and/or the primer for single base extension comprises the nucleic acid sequence set forth in SEQ ID NO: 135; and/or
the two amplification primers comprise nucleic acid sequences set forth in SEQ ID NOs: 83 and 84, respectively, and/or the primer for single base extension comprises the nucleic acid sequence set forth in SEQ ID NO: 136; and/or
the two amplification primers comprise nucleic acid sequences set forth in SEQ ID NOs: 85 and 86, respectively, and/or the primer for single base extension comprises the nucleic acid sequence set forth in SEQ ID NO: 137; and/or
the two amplification primers comprise nucleic acid sequences set forth in SEQ ID NOs: 87 and 88, respectively, and/or the primer for single base extension comprises the nucleic acid sequence set forth in SEQ ID NO: 138; and/or
the two amplification primers comprise nucleic acid sequences set forth in SEQ ID NOs: 89 and 90, respectively, and/or the primer for single base extension comprises the nucleic acid sequence set forth in SEQ ID NO: 139; and/or
the two amplification primers comprise nucleic acid sequences set forth in SEQ ID NOs: 91 and 92, respectively, and/or the primer for single base extension comprises the nucleic acid sequence set forth in SEQ ID NO: 140; and/or
the two amplification primers comprise nucleic acid sequences set forth in SEQ ID NOs: 93 and 94, respectively, and/or the primer for single base extension comprises the nucleic acid sequence set forth in SEQ ID NO: 141.

8. The set of isolated polynucleotides of claim 2, which is for detection of one or more SNPs associated with diabetes mellitus and/or a disease or condition related to diabetes mellitus.

9. The set of isolated polynucleotides of claim 8, which is for detection of one or more SNPs associated with type 2 diabetes mellitus, diabetic nephropathy, diabetic retinitis, diabetic cardiomyopathy (e.g., elderly diabetic cardiomyopathy), and/or drug resistance to an anti-diabetes medication.

10. The set of isolated polynucleotides of claim 9, wherein the SNPs associated with type 2 diabetes mellitus comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329.

11. The set of isolated polynucleotides of claim 9, wherein the SNPs associated with diabetic nephropathy comprise any one or more of rs2268388 and rs1801282.

12. The set of isolated polynucleotides of claim 9, wherein the SNPs associated with diabetic retinitis comprise any one or more of rs9565164, rs4668142, rs2380261, rs39059, rs17756941, rs245955, and rs245962.

13. The set of isolated polynucleotides of claim 9, wherein the SNPs associated with diabetic cardiomyopathy comprise any one or more of rs266729, rs3811951, rs156019, rs6234, rs1801282, and rs3856806.

14. The set of isolated polynucleotides of claim 9, wherein the SNPs associated with the drug resistance comprise any one or more of rs13266634, rs10494366, rs2237892, rs11977021, rs7651090, rs5219, rs7903146, rs1801282, rs6467136, rs2230806, rs11212617, and rs622342.

15. The set of isolated polynucleotides of claim 9, wherein the anti-diabetes medication comprises any one or more of Repaglinide, Rosiglitazone, Metformin, Gliclazide, and Pioglitazone.

16. The set of isolated polynucleotides of claim 15, wherein the SNPs associated with Repaglinide resistance comprise any one or more of rs13266634, rs10494366, rs2237892, rs11977021, rs7651090, rs5219, and rs7903146; wherein the SNPs associated with Rosiglitazone resistance comprise any one or more of rs13266634, rs2237892, rs1801282, rs6467136, and rs2230806; wherein the SNPs associated with Metformin resistance comprise any one or more of rs11212617 and rs622342; wherein the SNPs associated with Gliclazide resistance comprise rs5219; and/or wherein the SNPs associated with Pioglitazone resistance comprise rs1801282.

17. The set of isolated polynucleotides of claim 8, wherein the diabetes mellitus and/or disease or condition related to diabetes mellitus are in an East Asian population.

18. A kit comprising the set of isolated polynucleotides of claim 2.

19. The kit of claim 18, further comprising instructions for using the set of isolated polynucleotides to conduct a companion diagnostic test.

20. The kit of claim 19, wherein the companion diagnostic test is for monitoring treatment of diabetes mellitus and/or a disease or condition related to diabetes mellitus.

21. A method for risk assessment, diagnosis, prognosis and/or treatment monitoring of diabetes mellitus and/or a disease or condition related to diabetes mellitus in a subject, the method comprising:
detecting one or more single nucleotide polymorphisms (SNPs) in a biological sample from the subject, wherein the SNPs are selected from the group consisting of rs10229583, rs10811661, rs10886471, rs1111875, rs12742393, rs12779790, rs13266634, rs1535500, rs16856187, rs16861329, rs1801282, rs2237892, rs340874, rs3786897, rs4430796, rs5219, rs6017317, rs6467136, rs6815464, rs7041847, rs7172432, rs7612463, rs7651090, rs7756992, rs780094, rs7903146, rs8050136, rs831571, rs9470794, rs2268388, rs9565164, rs4668142, rs2380261, rs39059, rs17756941, rs245955, rs245962, rs266729, rs3811951, rs156019, rs6234, rs3856806, rs10494366, rs11977021, rs2230806, rs11212617, and rs622342, and/or complementary sequences thereof, and/or sequences in linkage disequilibrium therewith.

22. The method of claim 21, wherein a plurality of primers are used to detect the SNP or SNPs, and the plurality of primers comprise at least three of the nucleic acid sequences set forth in SEQ ID NOs: 1-141.

23. The method of claim 22, wherein the primer comprises two or more of the nucleic acid sequences set forth in SEQ ID NOs: 1-94.

24. The method of claim 22, wherein the primer comprises one or more of the nucleic acid sequences set forth in SEQ ID NOs: 95-141.

25. The method of claim 21, wherein the SNP or SNPs are detected using:
two amplification primers comprising nucleic acid sequences set forth in SEQ ID NOs: 1 and 2, respectively, and/or a primer for single base extension comprising the nucleic acid sequence set forth in SEQ ID NO: 95; and/or
two amplification primers comprising nucleic acid sequences set forth in SEQ ID NOs: 3 and 4, respectively, and/or a primer for single base extension comprising the nucleic acid sequence set forth in SEQ ID NO: 96; and/or
two amplification primers comprising nucleic acid sequences set forth in SEQ ID NOs: 5 and 6, respectively, and/or a primer for single base extension comprising the nucleic acid sequence set forth in SEQ ID NO: 97; and/or
two amplification primers comprising nucleic acid sequences set forth in SEQ ID NOs: 7 and 8, respectively, and/or a primer for single base extension comprising the nucleic acid sequence set forth in SEQ ID NO: 98; and/or
two amplification primers comprising nucleic acid sequences set forth in SEQ ID NOs: 9 and 10, respectively, and/or a primer for single base extension comprising the nucleic acid sequence set forth in SEQ ID NO: 99; and/or
two amplification primers comprising nucleic acid sequences set forth in SEQ ID NOs: 11 and 12, respectively, and/or a primer for single base extension comprising the nucleic acid sequence set forth in SEQ ID NO: 100; and/or
two amplification primers comprising nucleic acid sequences set forth in SEQ ID NOs: 13 and 14, respectively, and/or a primer for single base extension comprising the nucleic acid sequence set forth in SEQ ID NO: 101; and/or
two amplification primers comprising nucleic acid sequences set forth in SEQ ID NOs: 15 and 16, respectively, and/or a primer for single base extension comprising the nucleic acid sequence set forth in SEQ ID NO: 102; and/or
two amplification primers comprising nucleic acid sequences set forth in SEQ ID NOs: 17 and 18, respectively, and/or a primer for single base extension comprising the nucleic acid sequence set forth in SEQ ID NO: 103; and/or
two amplification primers comprising nucleic acid sequences set forth in SEQ ID NOs: 19 and 20, respectively, and/or a primer for single base extension comprising the nucleic acid sequence set forth in SEQ ID NO: 104; and/or
two amplification primers comprising nucleic acid sequences set forth in SEQ ID NOs: 21 and 22, respectively, and/or a primer for single base extension comprising the nucleic acid sequence set forth in SEQ ID NO: 105; and/or
two amplification primers comprising nucleic acid sequences set forth in SEQ ID NOs: 23 and 24, respectively, and/or a primer for single base extension comprising the nucleic acid sequence set forth in SEQ ID NO: 106; and/or
two amplification primers comprising nucleic acid sequences set forth in SEQ ID NOs: 25 and 26, respectively, and/or a primer for single base extension comprising the nucleic acid sequence set forth in SEQ ID NO: 107; and/or
two amplification primers comprising nucleic acid sequences set forth in SEQ ID NOs: 27 and 28, respectively, and/or a primer for single base extension comprising the nucleic acid sequence set forth in SEQ ID NO: 108; and/or
two amplification primers comprising nucleic acid sequences set forth in SEQ ID NOs: 29 and 30, respectively, and/or a primer for single base extension comprising the nucleic acid sequence set forth in SEQ ID NO: 109; and/or
two amplification primers comprising nucleic acid sequences set forth in SEQ ID NOs: 31 and 32, respectively, and/or a primer for single base extension comprising the nucleic acid sequence set forth in SEQ ID NO: 110; and/or
two amplification primers comprising nucleic acid sequences set forth in SEQ ID NOs: 33 and 34, respectively, and/or a primer for single base extension comprising the nucleic acid sequence set forth in SEQ ID NO: 111; and/or
two amplification primers comprising nucleic acid sequences set forth in SEQ ID NOs: 35 and 36, respectively, and/or a primer for single base extension comprising the nucleic acid sequence set forth in SEQ ID NO: 112; and/or
two amplification primers comprising nucleic acid sequences set forth in SEQ ID NOs: 37 and 38, respectively, and/or a primer for single base extension comprising the nucleic acid sequence set forth in SEQ ID NO: 113; and/or
two amplification primers comprising nucleic acid sequences set forth in SEQ ID NOs: 39 and 40, respectively, and/or a primer for single base extension comprising the nucleic acid sequence set forth in SEQ ID NO: 114; and/or
two amplification primers comprising nucleic acid sequences set forth in SEQ ID NOs: 41 and 42, respectively, and/or a primer for single base extension comprising the nucleic acid sequence set forth in SEQ ID NO: 115; and/or
two amplification primers comprising nucleic acid sequences set forth in SEQ ID NOs: 43 and 44, respectively, and/or a primer for single base extension comprising the nucleic acid sequence set forth in SEQ ID NO: 116; and/or
two amplification primers comprising nucleic acid sequences set forth in SEQ ID NOs: 45 and 46, respectively, and/or a primer for single base extension comprising the nucleic acid sequence set forth in SEQ ID NO: 117; and/or
two amplification primers comprising nucleic acid sequences set forth in SEQ ID NOs: 47 and 48, respectively, and/or a primer for single base extension comprising the nucleic acid sequence set forth in SEQ ID NO: 118; and/or
two amplification primers comprising nucleic acid sequences set forth in SEQ ID NOs: 49 and 50, respectively, and/or a primer for single base extension comprising the nucleic acid sequence set forth in SEQ ID NO: 119; and/or
two amplification primers comprising nucleic acid sequences set forth in SEQ ID NOs: 51 and 52, respectively, and/or a primer for single base extension comprising the nucleic acid sequence set forth in SEQ ID NO: 120; and/or
two amplification primers comprising nucleic acid sequences set forth in SEQ ID NOs: 53 and 54, respectively, and/or a primer for single base extension comprising the nucleic acid sequence set forth in SEQ ID NO: 121; and/or
two amplification primers comprising nucleic acid sequences set forth in SEQ ID NOs: 55 and 56, respectively, and/or a primer for single base extension comprising the nucleic acid sequence set forth in SEQ ID NO: 122; and/or
two amplification primers comprising nucleic acid sequences set forth in SEQ ID NOs: 57 and 58, respectively, and/or a primer for single base extension comprising the nucleic acid sequence set forth in SEQ ID NO: 123; and/or
two amplification primers comprising nucleic acid sequences set forth in SEQ ID NOs: 59 and 60, respectively, and/or a primer for single base extension comprising the nucleic acid sequence set forth in SEQ ID NO: 124; and/or
two amplification primers comprising nucleic acid sequences set forth in SEQ ID NOs: 61 and 62, respectively, and/or a primer for single base extension comprising the nucleic acid sequence set forth in SEQ ID NO: 125; and/or
two amplification primers comprising nucleic acid sequences set forth in SEQ ID NOs: 63 and 64, respectively, and/or a primer for single base extension comprising the nucleic acid sequence set forth in SEQ ID NO: 126; and/or
two amplification primers comprising nucleic acid sequences set forth in SEQ ID NOs: 65 and 66, respectively, and/or a primer for single base extension comprising the nucleic acid sequence set forth in SEQ ID NO: 127; and/or
two amplification primers comprising nucleic acid sequences set forth in SEQ ID NOs: 67 and 68, respectively, and/or a primer for single base extension comprising the nucleic acid sequence set forth in SEQ ID NO: 128; and/or
two amplification primers comprising nucleic acid sequences set forth in SEQ ID NOs: 69 and 70, respectively, and/or a primer for single base extension comprising the nucleic acid sequence set forth in SEQ ID NO: 129; and/or
two amplification primers comprising nucleic acid sequences set forth in SEQ ID NOs: 71 and 72, respectively, and/or a primer for single base extension comprising the nucleic acid sequence set forth in SEQ ID NO: 130; and/or
two amplification primers comprising nucleic acid sequences set forth in SEQ ID NOs: 73 and 74, respectively, and/or a primer for single base extension comprising the nucleic acid sequence set forth in SEQ ID NO: 131; and/or
two amplification primers comprising nucleic acid sequences set forth in SEQ ID NOs: 75 and 76, respectively, and/or a primer for single base extension comprising the nucleic acid sequence set forth in SEQ ID NO: 132; and/or
two amplification primers comprising nucleic acid sequences set forth in SEQ ID NOs: 77 and 78, respectively, and/or a primer for single base extension comprising the nucleic acid sequence set forth in SEQ ID NO: 133; and/or
two amplification primers comprising nucleic acid sequences set forth in SEQ ID NOs: 79 and 80, respectively, and/or a primer for single base extension comprising the nucleic acid sequence set forth in SEQ ID NO: 134; and/or
two amplification primers comprising nucleic acid sequences set forth in SEQ ID NOs: 81 and 82, respectively, and/or a primer for single base extension comprising the nucleic acid sequence set forth in SEQ ID NO: 135; and/or
two amplification primers comprising nucleic acid sequences set forth in SEQ ID NOs: 83 and 84, respectively, and/or a primer for single base extension comprising the nucleic acid sequence set forth in SEQ ID NO: 136; and/or
two amplification primers comprising nucleic acid sequences set forth in SEQ ID NOs: 85 and 86, respectively, and/or a primer for single base extension comprising the nucleic acid sequence set forth in SEQ ID NO: 137; and/or
two amplification primers comprising nucleic acid sequences set forth in SEQ ID NOs: 87 and 88, respectively, and/or a primer for single base extension comprising the nucleic acid sequence set forth in SEQ ID NO: 138; and/or
two amplification primers comprising nucleic acid sequences set forth in SEQ ID NOs: 89 and 90, respectively, and/or a primer for single base extension comprising the nucleic acid sequence set forth in SEQ ID NO: 139; and/or
two amplification primers comprising nucleic acid sequences set forth in SEQ ID NOs: 91 and 92, respectively, and/or a primer for single base extension comprising the nucleic acid sequence set forth in SEQ ID NO: 140; and/or
two amplification primers comprising nucleic acid sequences set forth in SEQ ID NOs: 93 and 94, respectively, and/or a primer for single base extension comprising the nucleic acid sequence set forth in SEQ ID NO: 141.

26. The method of claim 21, wherein the one or more SNPs are associated with type 2 diabetes mellitus, diabetic nephropathy, diabetic retinitis, diabetic cardiomyopathy (e.g., elderly diabetic cardiomyopathy), and/or drug resistance to an anti-diabetes medication.

27. The method of claim 26, wherein the SNPs associated with type 2 diabetes mellitus comprise any one or more of rs7756992, rs10811661, rs8050136, rs7041847, rs1111875, rs4430796, rs7651090, rs2237892, rs13266634, rs1801282, rs6017317, rs16856187, rs6467136, rs5219, rs1535500, rs6815464, rs12742393, rs10229583, rs3786897, rs831571, rs7903146, rs9470794, rs780094, rs10886471, rs12779790, rs340874, rs7612463, rs7172432, and rs16861329; wherein the SNPs associated with diabetic nephropathy comprise any one or more of rs2268388 and rs1801282; wherein the SNPs associated with diabetic retinitis comprise any one or more of rs9565164, rs4668142, rs2380261, rs39059, rs17756941, rs245955, and rs245962; wherein the SNPs associated with diabetic cardiomyopathy comprise any one or more of rs266729, rs3811951, rs156019, rs6234, rs1801282, and rs3856806; and/or wherein the SNPs associated with the drug resistance comprise any one or more of rs13266634, rs10494366, rs2237892, rs11977021, rs7651090, rs5219, rs7903146, rs1801282, rs6467136, rs2230806, rs11212617, and rs622342.

28. The method of claim 21, which further comprises treating diabetes mellitus and/or a disease or condition related to diabetes mellitus in the subject.

29. The method of claim 28, wherein the treatment of diabetes mellitus and/or a disease or condition related to diabetes mellitus in the subject is adjusted based on the SNP(s) detection result in the biological sample from the subject.
